SEQUENCE LISTING (155 sequences)

SEQ ID NO: 1 - DNA, 878 bp, Homo sapiens
  gene (1)...(878): Ferritin; Highly similar to FRIL_HUMAN Ferritin light chain (Ferritin L subunit) [H.sapiens], iron ion homeostasis
gtcccgcgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccatgagctcccagattcgtcagaattattccaccgacgtggaggcagccgtcaacagcctggtcaatttgtacctgcaggcctcctacacctacctctctctgggcttctatttcgaccgcgatgatgtggctctggaaggcgtgagccacttcttccgcgaattggccgaggagaagcgcgagggctacgagcgtctcctgaagatgcaaaaccagcgtggcggccgcgctctcttccaggacatcaagaagccagctgaagatgagtggggtaaaaccccagacgccatgaaagctgccatggccctggagaaaaagctgaaccaggcccttttggatcttcatgccctgggttctgcccgcacggacccccatctctgtgacttcctggagactcacttcctagatgaggaagtgaagcttatcaagaagatgggtgaccacctgaccaacctccacaggctgggtggcccggaggctgggctgggcgagtatctcttcgaaaggctcactctcaagcacgactaagagccttctgagcccagcgacttctgaagggccccttgcaaagtaatagggcttctgcctaagcctctccctccagccaataggcagctttcttaactatcctaacaagccttggaccaaatggaaataaagctttttgatgcaaaaaaaaaaaaaaaaaaa

SEQ ID NO: 2 - DNA, 1567 bp, Homo sapiens
  gene (1)...(1567): FKBP8; FK506 binding protein 8, 38kDa
gggtgaggaggaagaggaggaggaagaggaggaagaggatgacctgagtgagctgccaccgctggaggacatgggacaacccccggcggaggaggctgagcagcctggggccctggcccgagagttccttgctgccatggagcccgagcccgccccagccccggccccagaagagtggctggacattctggggaacgggctgttgaggaagaagacgctggtcccagggccgccaggttcgagccgcccggtcaagggccaggtggtcaccgtacatctgcagacgtcgctggagaatggcacacgggtgcaggaggagccggagctggtgttcactctgggtgactgtgacgtcatccaggccctggatctcagtgtcccactcatggacgtgggggagacggccatggtcactgctgactccaagtactgctacggcccccaaggcagcaggagcccatacatccccccgcacgcggccctgtgcctggaggtgaccctgaagacggctgtggacgggcctgacctggagatgctcacggggcaggagcgcgtggccctggccaaccggaagcgggagtgcggcaacgcccactaccagcgggcggacttcgtcctggccgccaactcctacgacctcgccatcaaggctatcacctccagcgccaaagtggacatgacgttcgaggaggaggcacagctcctgcagttgaaggtgaagtgtctgaacaacctggcggcctcgcagctgaagctcgaccactaccgcgcagccctgcgctcctgcagccttgtgctggagcaccagccagacaacatcaaggctctcttccgcaagggcaaggtgctggcccagcagggggagtacagtgaggccatccccatcctgagggcagccctgaagctggaaccttccaacaagacgatccacgcagagctctcaaagctggtgaagaagcatgcggcgcagcggagcacggagaccgccttgtaccggaaaatgctgggcaaccccagccggctgcctgctaagtgccctggcaagggtgcctggtccatcccatggaagtggctgtttggggcgactgctgttgccttggggggtgtggcactctctgtggtcatcgctgccaggaactgaccacctaggtggctgccaccccctctgcacaccatggaccctgccctgcgctccccaactcccccaggctccctgtccactgccctccctggtctggccccctcctccgggttaggggagcaaggattgggggtcgtgcagcccagccagcaggagggactgaggccctctaggaggaaagcccagagggagggggccctcattccttcagacccagttttcccccaccctccttaccccgctgggctaggtctccgccagggctggcctcagtttctcctcaacaggcctgggggcagcccttcccctgcctagtccccgcctgagtgccagccccccaccccgcctgccgccccctgtccaggttccctccccgccacagtgaaataaagcatcccaccctgcaaaaaaaaaaaaaaaaaaaaa

SEQ ID NO: 3 - DNA, 1596 bp, Homo sapiens
  gene (1)...(1596): TUBA3 tubulin, alpha, ubiquitous
tgtcggggacggtaaccgggacccgtgctctgctcctgtcgccttcgcctcctgaatccctagccatatgcgtgagtgcatctccatccacgttggccaggctggtgtccagattggcaatgcctgctgggagctctactgcctggaacacggcatccagcccgatggccagatgccaagtgacaagaccattgggggaggagatgactccttcaacaccttcttcagtgagacgggcgctggcaagcacgtgccccgggctgtgtttgtagacttggaacccacagtcattgatgaagttcgcactggcacctaccgccagctcttccaccctgagcagctcatcacaggcaaggaagatgctgccaataactatgcccgagggcactacaccattggcaaggagatcattgaccttgtgttggaccgaattcgcaagctggctgaccagtgcacccgtcttcagggcttcttggttttccacagctttggtgggggaactggttctgggttcacctccctgctcatggaacgcctgtcagttgattatggcaagaaatccaagctggagttctccatttacccggcaccccaggtttccacagctgtagttgagccctacaactccatcctcaccacccacaccaccctggagcactctgattgtgccttcatggtagacaatgaggccatctatgacatctgtcgtagaaacctcgatatcgagcgcccaacctacactaaccttaaccgccttattagccagattgtgtcctccatcactgcttccctgagatttgatggagccctgaatgttgacctgacagaattccagaccaacctggtcccctacccccgcatccacttccctctggccacatatgcccctgtcatctctgctgagaaagcctaccatgaacagctttctgtagcagacatcaccaatgcttgctttgagccagccaaccagatggtgaaatgtgaccctggccatggtaaatacatggcttgctgcctgttgtaccgtggtgacgtggttcccaaagatgtcaatgctgccattgccaccatcaaaaccaagcgcacgatccagtttgtggattggtgccccactggcttcaaggttggcatcaactaccagcctcccactgtggtgcctggtggagacctggccaaggtacagagagctgtgtgcatgctgagcaacaccacagccattgctgaggcctgggctcgcctggaccacaagtttgacctgatgtatgccaagcgtgcctttgttcactggtacgtgggtgaggggatggaggaaggcgagttttcagaggcccgtgaagatatggctgcccttgagaaggattatgaggaggttggtgtggattctgttgaaggagagggtgaggaagaaggagaggaatactaattatccattccttttggccctgcagcatgtcatgctcccagaatttcagcttcagcttaactgacagatgttaaagctttctggttagattgttttcacttggtgatcatgtcttttccatgtgtacctgtaatatttttccatcatatctcaaagtaaagtcattaacatca

SEQ ID NO: 4 - DNA, 1800 bp, Homo sapiens
  gene (1)...(1800): TTS-2.2; transport-secretion protein 2.2
gacagcgtctccgcctccgccggcggagaccccaaggtatcgagactgcgggacccactgcccgcaggacatcgagtcacgatgttcacgagggagaccaagtggaacatctcattcgctggctgcggcttcctcggggtctaccacattggcgtggcctcctgcctccgtgagcacgcgcccttcctggtggccaacgccactcacatctacggagcctcggcaggggcgctcaccgccacagcgctggtcactggggcctgcctgggtgaagcaggtgccaacattattgaggtgtccaaggaggcccggaagcggttcctgggtcctctgcatccctccttcaacctggtgaagaccatccgtggctgtctactaaagaccctgcctgctgattgccatgagcgcgccaatggacgcctgggcatctccctgactcgtgtttcagacggagagaacgtcatcatatcccactttagctccaaggatgagctcatccaggccaatgtctgcagcacatttatcccggtgtactgtggcctcattcctcctaccctccaaggggtgcgctatgtggatggcggcatttcagacaacttgccactttatgagctgaagaataccatcacagtgtccccattctcaggcgagagtgacatctgccctcaggacagctccaccaacatccacgagcttcgcgtcaccaacaccagcatccagttcaaccttcgcaatctctaccgcctctcgaaggctctcttcccgccagagcccatggtcctccgagagatgtgcaaacagggctacagagatggacttcgattccttaggaggaatgccctgctggaggcctgtgtggaaccaaaggacctgatgaccaccctttccaacatgctaccagtgcgcctggcaacggccatgatggtgccctatactctgccgctggagagtgcagtgtccttcaccatccgcttgttggagtggctgcctgatgtccctgaagatatccggtggatgaaagagcagacgggtagcatctgccagtatctggtgatgagggccaagaggaaattgggtgaccatctgccttccagactgtctgagcaggtggaactgcgacgtgcccagtctctgccctctgtgccactgtcttgcgccacctacagtgaggccctacccaactgggtacgaaacaacctctcactgggggacgcgctggccaagtgggaagaatgccagcgtcagctactgctgggtctcttctgcaccaatgtggccttcccgccggatgccttgcgcatgcgcgcacctgccagccccactgccgcagatcctgccaccccacaggatccacctggcctcccgccttgctgagaatcaccattcccacatcgcccggctaccagccaagctccaagttgtcctgccccactaagaggagccccggggtggaacaagatcctgtctgccccggctctcccccttacatgctgtggaatgaggacataggaccctgcacagctgcaagtgggctttcgatgtgaaacctttcaccagccactcactatgctactcctggtggggagggatggggagtcgccctcccccggagcccacagagccctcccccgtcacgtcacctgtgccttactcctgcccaccaccttttcagtgcagggtcagtcttaagaactccacatctgctgctgctccctggtgtccaagtttccttgcagagtgtgtgaagaattatttatttttgccaaagcagatctaataaaagccacagctcagcttctg

SEQ ID NO: 5 - DNA, 1656 bp, Homo sapiens
  gene (1)...(1656): UBXD1; UBX domain-containing 1
ttttcttccgggggctggtctccggcggccccgtccccgactgggccccgtgcccccccgcccccgcggccccccgccgccgggccagccgccaccatgaagaaattctttcaggagttcaaggccgacatcaagttcaagagcgcgggacccggtcagaagctcaaagagtccgtgggggaaaaggcccacaaagagaagcccaaccagccagcccccaggccgccccgccagggacccaccaatgaggcacagatggcagccgctgctgccctagcccggctggagcagaagcagtcccgggcctggggccccacatcgcaggacaccatccgaaaccaggtgagaaaggaacttcaagccgaagccaccgtcagcgggagccccgaggccccagggaccaacgtggtatctgagcccagagaggaaggctctgcccacctggctgtgcctggcgtgtacttcacctgtccgctcactggggccaccctgaggaaggaccagcgggacgcctgcatcaaggaggccattctcttgcacttctccaccgacccagtggccgcctccatcatgaagatctacacgttcaacaaagaccaggaccgggtgaagctgggtgtggacaccattgccaagtacctggacaacatccacctgcaccccgaggaggagaagtaccggaagatcaagctgcagaacaaggtgtttcaggagcgcattaactgcctggaagggacccacgagttttttgaggccattgggttccagaaggtgttgcttcccgcccaggatcaggaggaccccgaggagttctacgtgctgagcgagaccaccttggcccagccccagagcctggagaggcacaaggaacagctgctggctgcggagcccgtgcgcgccaagctggacaggcagcgccgcgtcttccagccctcgcccctggcctcgcagttcgaactgcctggggacttcttcaacctcacagcagaggagatcaagcgggagcagaggctcaggtccgaggcggtggagcggctgagcgtgctgcggaccaaggccatgcgggagaaggaggagcagcgggggctgcgcaagtacaactacacgctgctgcgcgtgcgcctccccgatggctgcctcctgcagggcactttctacgctcgggagcggctgggggcggtgtacgggttcgtccgggaggccctgcagagcgactggctgccttttgagctgctggcctcgggagggcagaagctgtccgaggacgagaacctggccttgaacgagtgcgggctggtgccctctgccctcctgaccttctcgtgggacatggctgtgctggaggacatcaaggccgcgggggccgagccggactccatcctgaaacccgagctcctgtcagccatcgagaagctcttgtgaaataaaagcagggttggcctcagccctgtgggtctgtctcatgctctccctgttcctctccccgccaccccagggcctccaagccacctctggaaatacttggctctgccccatgggcacgggaggggcgccagccgtggagctgtggaattgggccccgtggcagagcccccatcccttgggggctgtggggatgcgcccaagcccccgagggagaggcctggggacaccaac

SEQ ID NO: 6 - DNA, 1745 bp, Homo sapiens
  gene (1)...(1745): LOC51257; Hypothetical protein LOC51257
catttatccagcagtgaactgtcctagcgcaagagttagtaattgctcccctgttccttcacctccccactttggagctcagatttgtttttttgtttgtttgtttgcttgctttcttttgttctgttttagagactggagactgggtcttgctctgttacccaggctggagtgcagtggtgtgatcatagctcactacagccttgaactcctgggctcaagaggttgaggctccctcctcagcctcccaagtagctgggactacaggctttcagcaccatgcctggctaattcaaaaaaaccttcagagagatagggtctctctatgttgccctagctcgtctcaaactcctggcctcaagtgatcctcctgcttggacctcccaaagcgctgggattacaggctcctggaaccatgggcctcaggccctgaggatacggggctcccggtggccatgacgacgggtgactgctgccacctccccggctccctgtgtgactgctccggcagccctgccttctccaaggtcgtggaggctacgggcctcggaccgccccagtatgtggcacaggtgacttcaagggatggccggctcctctccaccgtcatccgtaccttggacacaccgagtgatggtcctttctgccggatctgccatgagggagcgaacggggagtgcttgctgtccccgtgtggctgcaccggcacgctgggtgccgtgcataagagctgtctggagaagtggctttcctcatctaacaccagctactgcgagctgtgccacacggagtttgcagtggagaaacggcctcgacccctcacagagtggctgaaggacccggggccgcggacggagaagcggacactgtgctgcgacatggtgtgtttcctgttcatcacaccgctggccgccatctcaggctggttgtgcctgcgcggggcccaggaccacctccggctccacagccagctggaggccgtgggtctcattgccctcaccatcgccctcttcaccatctatgtcctctggacgctggtctccttccgctaccactgccagctgtactccgagtggagaaagaccaaccagaaagttcgcctgaagatccgggaggcggacagccccgagggcccccagcattctccactggcagctggactcctgaagaaggtggcagaggagacaccagtatgaatgctgggctctccggaccctgcagcagagaggccagaggtagctggtgataccctgtcctgtggaaggacttccacttcaacacttccacttcaacagttcccgcacggcctgaacgcttcttaggccaagagacaccatgcggagcctagtctgtgatcctgtgtgaagatattttcagggttttttttttttttttttttgcatatggaggacaggtggacatggtcctgagctctggacggagcaggcaccctgatctcattctgaggtccacatggcaccttctgggccagcagctgtggccggtgtatcaagggcgcccttaaagctggaacattccagcaagcttcttgcgcttctctgcacccggcaggcccactttcctggcaccctcgactttatataaaagttgcactgcgtttcaaaaacccaccctgaatgaataaaaggagccctggctggacaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - DNA, 2700 bp, Homo sapiens
  gene (1)...(2700): ITRPK1; Inositol 1,3,4-triphosphate 5/6 kinase
cgcagaggcaccgccccaagtttgttgtgaccggcgggggacgccggtggtggcggcagcggcggctgcgggggcaccgggccgcggcgccaccatggcggtgcgacaggcgctgggccgcggcctgcagctgggtcgagcgctgctgctgcgcttcacgggcaagcccggccgggcctacggcttggggcggccgggcccggcggcgggctgtgtccgcggggagcgtccaggctgggccgcaggaccgggcgcggagcctcgcagggtcgggctcgggctccctaaccgtctccgcttcttccgccagtcggtggccgggctggcggcgcggttgcagcggcagttcgtggtgcgggcctggggctgcgcgggcccttgcggccgggcagtctttctggccttcgggctagggctgggcctcatcgaggaaaaacaggcggagagccggcgggcggtctcggcctgtcaggagatccaggcaatttttacccagaaaagcaagccggggcctgacccgttggacacgagacgcttgcagggctttcggctggaggagtatctgatagggcagtccattggtaagggctgcagtgctgctgtgtatgaagccaccatgcctacattgccccagaacctggaggtgacaaagagcaccgggttgcttccagggagaggcccaggtaccagtgcaccaggagaagggcaggagcgagctccgggggcccctgccttccccttggccatcaagatgatgtggaacatctcggcaggttcctccagcgaagccatcttgaacacaatgagccaggagctggtcccagcgagccgagtggccttggctggggagtatggagcagtcacttacagaaaatccaagagaggtcccaagcaactagcccctcaccccaacatcatccgggttctccgcgccttcacctcttccgtgccgctgctgccaggggccctggtcgactaccctgatgtgctgccctcacgcctccaccctgaaggcctgggccatggccggacgctgttcctcgttatgaagaactatccctgtaccctgcgccagtacctttgtgtgaacacacccagcccccgcctcgccgccatgatgctgctgcagctgctggaaggcgtggaccatctggttcaacagggcatcgcgcacagagacctgaaatccgacaacatccttgtggagctggacccagacggctgcccctggctggtgatcgcagattttggctgctgcctggctgatgagagcatcggcctgcagttgcccttcagcagctggtacgtggatcggggcggaaacggctgtctgatggccccagaggtgtccacggcccgtcctggccccagggcagtgattgactacagcaaggctgatgcctgggcagtgggagccatcgcctatgaaatcttcgggcttgtcaatcccttctacggccagggcaaggcccaccttgaaagccgcagctaccaagaggctcagctacctgcactgcccgagtcagtgcctccagacgtgagacagttggtgagggcactgctccagcgagaggccagcaagagaccatctgcccgagtagccgcaaatgtgcttcatctaagcctctggggtgaacatattctagccctgaagaatctgaagttagacaagatggttggctggctcctccaacaatcggccgccactttgttggccaacaggctcacagagaagtgttgtgtggaaacaaaaatgaagatgctctttctggctaacctggagtgtgaaacgctctgccaggcagccctcctcctctgctcatggagggcagccctgtgatgtccctgcatggagctggtgaattactaaaagaacttggcatcctctgtgtcgtgatggtctgtgaatggtgagggtgggagtcaggagacaagacagcgcagagagggctggttagccggaaaaggcctcgggcttggcaaatggaagaacttgagtgagagttcagtctgcagtcctgtgctcacagacatctgaaaagtgaatggccaagctggtctagtagatgaggctggactgaggaggggtaggcctgcatccacagagaggatccaggccaaggcactggctgtcagtggcagagtttggctgtgacctttgcccctaacacgaggaactcgtttgaagggggcagcgtagcatgtctgatttgccacctggatgaaggcagacatcaacatgggtcagcacgttcagttacgggagtgggaaattacatgaggcctgggcctctgcgttcccaagctgtgcgttctggaccagctactgaattattaatctcacttagcgaaagtgacggatgagcagtaagtaagtaagtgtggggatttaaacttgagggtttccctcctgactagcctctcttacaggaattgtgaaatattaaatgcaaatttacaactgcagatgacgtatgtgccttgaactgaatatttggctttaagaatgattcttatactctgaaggtgagaatattttgtgggcaggtatcaacattggggaagagatttcatgtctaactaactaactttatacatgatttttaggaagctatgcctaaatcagcgtcaacatgcagtaaaggttgtcttcaactgaaaaaaaaaaaaaaaaaaaaaaaaaaaaaaaaaaaaaaaaaa

SEQ ID NO: 8 - DNA, 3139 bp, Homo sapiens
  gene (1)...(3139): PINK1; PTEN induced putative kinase 1
ggccgaggaggaagtggcggcggcggcggcgggactgcgcgccccagctccgatccccgttccgcgtccccgccgccgggaggaggtgcccactcgctcgcggcgcgcgccggccgccagactcggcctgtgggcgatttcctccggacccaggctccccgcccgaggaggaagatgcagacctttctgaaagggaagagagttggctactggctgagcgagaagaaaatcaagaagctgaatttccaggccttcgccgagctgtgcaggaagcgagggatggaggttgtgcagctgaaccttagccggccgatcgaggagcagggccccctggacgtcatcatccacaagctgactgacgtcatccttgaagccgaccagaatgatagccagtccctggagctggtgcacaggttccaggagtacatcgatgcccaccctgagaccatcgtcctggacccgctccctgccatcagaaccctgcttgaccgctccaagtcctatgagctcatccggaagattgaggcctacatggaagacgacaggatctgctcgccacccttcatggagctcacgagcctgtgcggggatgacaccatgcggctgctggagaagaacggcttgactttcccattcatttgcaaaaccagagtggctcatggcaccaactctcacgagatggctatcgtgttcaaccaggagggcctgaacgccatccagccaccctgcgtggtccagaatttcatcaaccacaacgccgtcctgtacaaggtgttcgtggttggcgagtcctacaccgtggtccagaggccctcactcaagaacttctccgcaggcacatcagaccgtgagtccatcttcttcaacagccacaacgtgtcaaagccggagtcgtcatcggtcctgacggagctggacaagatcgagggcgtgttcgagcggccgagcgacgaggtcatccgggagctctcccgggccctgcggcaggcactgggcgtgtcactcttcggcatcgacatcatcatcaacaaccagacagggcagcacgccgtcattgacatcaatgccttcccaggctacgagggcgtgagcgagttcttcacagacctcctgaaccacatcgccactgtcctgcagggccagagcacagccatggcagccacaggggacgtggccctgctgaggcacagcaagcttctggccgagccggcgggcggcctggtgggcgagcggacatgcagcgccagccccggctgctgcggcagcatgatgggccaggacgcgccctggaaggctgaggccgacgcgggcggcaccgccaagctgccgcaccagagactcggctgcaacgccggcgtgtcgcccagcttccagcagcattgtgtggcctccctggccaccaaggcctcctcccagtagccacggagccgggacccagaggggcagcgcagggcgcagagcacacccgctgggccagcagctcccaacggcgatgctactactaagaatccccagtgatctgattcttctgttttttaatttttaacctgattctctgatgtcatgatctaaatgaggggtagtgggggatggaagagagtaccaggtggtccaccgttggggagtggggccgtccgcctgctctctactgtgcagacctcctaactgagtttacacacgcttgtgtttgcaacactaggtctggatgggaggtgaggggtgtgcgtatggctgccatgccggtgtctgtgcacatccctgtctgttggtctccatggccactgtggaccgggacccttgggggaagcctgcccatgtcgggctgtgggaggctgatcggtgcatgtgagagtggcttcccttctgcctgactccccactccctgacctgccccttccttgtttttcctcctactggtctccaccaaggctttgttagcccccaccctgcctggtgtgcagctaacccctccctccccacagccagaggaggccacagacccctcaggggagttccgcgctggtgtctgggctgtgctccctcactaaagggaaggaaaggaagctgggcgtcctctgggccccccaacacacgtcccatttagccctgcacagcggtctccttcccctaagccagcactgctgctccctggagcccgggaaggaggctgcctggctggaggcccgagccgatggcgcctgtgctgaggatttgtgctgtgatttgggcaaatcattccaggtctttgggcctccacccccttgtctctagtggacatttgagatcagagagcaccacagggctggctttgtgccctaacccctgggatgcagcctgcctttccataaagtcacctaggtgaggataggcgcgggagcctcggcatgacaccatggagatcggggccctcttcccagtgggttcactccttttcacacctgctgggtccctcctcacccagcaggcctggtccacctctcattgcaagccccaagcactgagccagcaggtgccagggagccacccgccccccatcgcttctgcacacctcagactcaccccatcaccttggcagcaaagcactggctctgccgtctgacccctgatccaggcagccccctctgcagagaaaagggttggggagaagcctctgcagtcctggaagatgtggggtgctgggtgagaggcatcagcccccacaagtatgtttttgtgtcttaagatagcagtttactttgaaaaagtgaaaaaggcttccgggctgtcctctgcccagtgagatggaggacgctagagaaagtgctgagtgtcccgagagaggcccccgagccagtgcatggaggtccttcggcctggctcagctgggctgcaggatgcccactttgaggagggaggcacagggcttgggcgaggggcagaggccatcagaactgcccggcttttttggaaactgaggacccaacaactaaccacgtttacacgacttgagttttgaaccccgattaatgtctgtacgtcacctttcctagttctgaccctgagccctggggaacaggaaagcgtggctggcctcttgcactgctttgtctccaaaataaactactgaaatcaaaccgcaaaaaaaaaaaaaaaaaaaaaa

SEQ ID NO: 9 - DNA, 765 bp, Homo sapiens
  gene (1)...(765): PMS2L5; postmeiotic segregation increased 2-like 5
cggacgcgtgggtcgggcactttcccagtccccgaggcggatccggtgttgcatccttggagagagctgagagctggagtacagaacctgctaaggccatcaaacctattgatcggaagtcagtccatcagatttgctctgggccggtagtactgagtctaagcactgcggtgaagaagatggtagaaaacagtctggatgctggtgccactaatattgatctaaagcttaaggactatggaatggatctcattgaagtttcaggcaatggatgtggggtagaagaagaaaacttcgaaggcttaatgatgtcaccatttctacctgccacgtatcggcgaaggttgggactcgactggtgtttgatcacgatgggaaaatcatccagaaaaccccctacccccaccccagagggaccacagtcagcgtgaagcagttattttctacgctacctgtgcgccataaggaatttcaaaggaatattaagaagaaacgtgcctgcttccccttcgccttctgccgtgattgtcagttccttgagggctccccagccatgcttcctgtacagcctgcaaaactgactcctagaagtaccccaccccacccctgctccttggaggacaacgtgatcactgtattcagctctgtcaagaatggtccaggttcttctagatgatctgcacaaatggttcctctcctccttcctgatgtctgccattagcattggaataaagttcctgctgaaaatccaaaaaaaaaaaaaaa

SEQ ID NO: 10 - DNA, 5449 bp, Homo sapiens
  gene (1)...(5449): RXRA; retinoid X receptor, alpha
gcgccgggggccgccgcgcccgccgcccgctgcctgcgccgccggccgggcatgagttagtcgcagacatggacaccaaacatttcctgccgctcgatttctccacccaggtgaactcctccctcacctccccgacggggcgaggctccatggctgccccctcgctgcacccgtccctggggcctggcatcggctccccgggacagctgcattctcccatcagcaccctgagctcccccatcaacggcatgggcccgcctttctcggtcatcagctcccccatgggcccccactccatgtcggtgcccaccacacccaccctgggcttcagcactggcagcccccagctcagctcacctatgaaccccgtcagcagcagcgaggacatcaagccccccctgggcctcaatggcgtcctcaaggtccccgcccacccctcaggaaacatggcttccttcaccaagcacatctgcgccatctgcggggaccgctcctcaggcaagcactatggagtgtacagctgcgaggggtgcaagggcttcttcaagcggacggtgcgcaaggacctgacctacacctgccgcgacaacaaggactgcctgattgacaagcggcagcggaaccggtgccagtactgccgctaccagaagtgcctggccatgggcatgaagcgggaagccgtgcaggaggagcggcagcgtggcaaggaccggaacgagaatgaggtggagtcgaccagcagcgccaacgaggacatgccggtggagaggatcctggaggctgagctggccgtggagcccaagaccgagacctacgtggaggcaaacatggggctgaaccccagctcgccgaacgaccctgtcaccaacatttgccaagcagccgacaaacagcttttcaccctggtggagtgggccaagcggatcccacacttctcagagctgcccctggacgaccaggtcatcctgctgcgggcaggctggaatgagctgctcatcgcctccttctcccaccgctccatcgccgtgaaggacgggatcctcctggccaccgggctgcacgtccaccggaacagcgcccacagcgcaggggtgggcgccatctttgacagggtgctgacggagcttgtgtccaagatgcgggacatgcagatggacaagacggagctgggctgcctgcgcgccatcgtcctctttaaccctgactccaaggggctctcgaacccggccgaggtggaggcgctgagggagaaggtctatgcgtccttggaggcctactgcaagcacaagtacccagagcagccgggaaggttcgctaagctcttgctccgcctgccggctctgcgctccatcgggctcaaatgcctggaacatctcttcttcttcaagctcatcggggacacacccattgacaccttccttatggagatgctggaggcgccgcaccaaatgacttaggcctgcgggcccatcctttgtgcccacccgttctggccaccctgcctggacgccagctgttcttctcagcctgagccctgtccctgcccttctctgcctggcctgtttggactttggggcacagcctgtcactgctctgcctaagagatgtgttgtcaccctccttatttctgttactacttgtctgtggcccagggcagtggctttcctgaggcagcagccttcgtggcaagaactagcgtgagcccagccaggcgcctccccaccgggctctcaggacaccctgccacaccccacggggcttgggcgactacagggtcttcgggccccagccctggagctgcaggagttgggaacggggcttttgtttccgttgctgtttatcgatgctggttttcagaattcctgtgtggccctcctgtctggagtgacatcttcatctgctctgaatactggtgcccagccagcccgtgacagcttccccctaatcaggaggggacagctgggggcgcaagctggtgtgtcatcagcaaagacctcagccgcctcggggatgagaggggactcgtggggcaagcaagctgccctgtgctctgagtgagggggaaggtagcccctttttccaaagataactcacagttttgccctcgagccaatgagaacatgagctgccctctgtgcaaggtttcggggccacctccaggctgcaggggcgggtcactcacccccctgttttctctctgccttggtgttctggtttcagactcccgactccccgttcagaccagagtgccccggcccctccccagcctgagtcttctccttgctctgcggggtgggctgaggcttgtccttgtttcctgcagggctggccctggctcgggcagggtggggcatcaccacctcactggccttgctggaggcacagggctctgcggacctgcagccatctgtgaggcccgcggggatgggaggggaggagggtggcctgttggtttccctcagagggggcaggtggcctggagagagaggggctcaggaactgggagcctcgtgggtggggcagatgctccgcggcctggagtggctctgccggggcattggtgggacccctgctcaggccttctctctggctgccagttgtgtctaaaagactcttggaatctgagaacccggagtcgcagcgccctcgggcctgggccacacgcaggccctggtgggaccacccagcctggtattgtccacggacagcgttgttcacccagagccttacttgggagcctcactgaacgcctgctctggttgaaggtggggtgggggcggggcttggggcctccctggctcagcccagtgcggcctggcgctcctcccgcaggctctgcccccgggctccggtggtgcggggccctctcaggttgaactcgcctcttttgcactggaaggccctccctttggcctgagtacttttcccgttcacgcctcagtcccgtggacccagcctttgtcagtggcaggtgcctgaacagagggtggatgggggggataccggagggggtcttgtcttcccagccgcagtctaggaatgatgcgggggggtggacgccttctccatagtctttccccacctggagcaggggcttcctcagtggtgaggggagctgcctacaggttggaccgggaggcagtggcttggagaggcagctttccagccttggtggggaagaaagtgtccattctttgccttcctggagctcccagccagagctgagcttaggcacccgagtggagcctgcagctgagtctgtgcccgagacaggctgtcagagattccagaagcctctcctccccgccgccctccacccctgcctttcagcgttgtggatccctagaggtggccccctgcccgatccaccgtcctgaggcagagtgttgagcctcatacctgtaccaggtccccggccagctgggcccctcccaggcactgccaggaagccccagctgcccctggcgggtgtggtggaaatggcaggagggtgcaggtactcttggggccccagcggtgggagtgcaaaagacccaacgccaacacctggtgccttttgcagccagcgcccacccatccgtgcccggacccttgggaatgcccgcggctccagaggaaaaagcccagggacggggcctccgttgcggggggtcggctgcttcttgggaactttgtcgtttccggcgctggctggctggctggctgtaaagcactgaagccccccggccgccaacccctgaaagcagaacctggcctccctggccacagcagccttacccaccgctctacgtgtcccgggcacttcccgcagccttcccgtccctttctcatcggccttgtagttgtacagtgctgttggtttgaaaaggtgatgtgtggggagtgcggctcatcactgagtagagaggtagaatttctatttaaccagacctgtagtagtattaccaatccagttcaattaaggtgattttttgtaattattattattttggtgggacaatctttaattttctaaagatagcactaacatcagctcattagccacctgtgcctgtccccgccttggcccggctggatgaagcggcttccccgcagggcccccacttcccagtggctgcttcctggggacccagggcaccccggcaccttcaggcacgctcctcagctggtcacctcccggctttgccgttcagatggggctcctgaggctcaggagtgaagatgccacagagccgggctcccctaggctgcgtcgggcatgcttggaagctggcctgccaggaccttccaccctggggcctgtgtcagccgccggccctccgcaccctggaagcacacggcctctgggaaggacagccctgaccttcggttttccgagcacggtgtttcccaagaattctgggctggcggcctggtggcagtgctggagatgaccccgagcccctccccgtggggcacccaggagggccctgccggaatgtgcagcctgtgggtagtcggctggtgtccctgtcgtggagctggggtgcgtgatctggtgctcgtccacgcaggtgtgtggtgtaaacatgtatgtgctgtacagagagacgcgtgtggagagagccgcacaccagcgccacccaggaaaggcggagcggttaccagtgttttgtgtttatttttaatcaagacgtttcccctgttttcctataaatttgcttcgtgtaagcaagtacataaggaccctcctttggtgaaatccgggttcgaatgaatatctcaaggcaggagatgcatctattttaagatgctttggagcagacagctttagccgttcccaatccttagcaatgccttagctgggacgcatagctaatactttagagaggatgacagatccataaagagagtaaagataagagaaaatgtctaaagcatctggaaaggtaaaaaaaaaaaatctatttttgtacaaatgtaattttatccctcatgtatacttggatatggcggggggagggctgggactgtttcgtttctgcttctagagattgaggtgaaagcttcgtccgagaaacgccaggacagacgatggcagaggagagggctcctgtgacggcggcgaggcttgggaggaaaccgccgcaatgggggtgtcttccctcggggcaggagggtgggcctgaggctttcaagggttttcttccctttcgagtaatttttaaagccttgctctgttgtgtcctgttgccggctctggccttcctgtgactgactgtgaagtggcttctccgtacgattgtctctgaaacatcgtggcctcaggtgccagggtttgatggacagtagcattagaattgtggaaaaggaacacgcaaagggagaagtgtgagaggagaaacaaaatatgagcgtttaaaatacatcgccattcagttcgttaaaaaaaaaaaaaaaaaaaaaaaaa

SEQ ID NO: 11 - DNA, 567 bp, Homo sapiens
  gene (1)...(567): GCN5L1; GCN5 general control of amino-acid synthesis 5-like 1; biogenesis of lysosome-related organelles complex-1, subunit 1 (BLOC1S1)
cagcgggcacgtgacatggccccggggagccgaggtgagcgtccagcttccggagccggagggggcccggcgtacccagcccccagcccgacgtgaccatgctgtcccgcctcctaaaagaacaccaggccaagcagaatgaacgcaaggagctgcaggaaaagaggaggcgagaggctatcactgcagcgacctgcctgacagaagctttggtggatcacctcaatgtgggtgtggcccaggcctacatgaaccagagaaagctggaccatgaggtgaagaccctacaggtccaggctgcccaatttgccaagcagacaggccagtggatcggaatggtggagaacttcaaccaggcactcaaggaaattggggatgtggagaactgggctcggagcatcgagctggacatgcgcaccattgccactgcactggaatatgtctacaaagggcagctgcagtctgccccttcctagcccctgttccctccccaaaccctatccctcctacctcacccgcagggggaaggagggaggctgacaagccttgaataaaacacaagcctcc

SEQ ID NO: 12 - DNA, 7222 bp, Homo sapiens
  gene (1)...(7222): CABIN1; calcineurin binding protein 1
agccggacggcgcggtggtcgggacagactggccgttgctgtggagacgcctggagagttgtggactggggcaacctttgccagtgatgagaagtgatgctcgtggcagtgctgaatctctctgaatatgattcgaattgcagccttaaatgccagctccaccattgaggatgatcatgaaggaagctttaaaagtcacaaaacccagacaaaggaggctcaggaagcagaggcttttgcattgtaccacaaggcccttgatctgcagaaacatgaccggtttgaggagtctgccaaagcctaccatgagctcttggaggcgagcctgctgcgggaggcagtttcatccggtgatgagaaagaggggttgaaacaccctgggctgatactgaaatattccacttataagaacttggcccagctggcagcccagcgggaggatctggagacagccatggagttctacttagaggcagtgatgctggactccacagatgtcaacctctggtataagattggacatgtggccctgaggctcatccggatccccctggctcgccatgcttttgaggaagggctgcggtgcaatcctgaccactggccctgtttggataacctaatcactgtcctgtacaccctcagtgattacacaacatgtctgtacttcatctgcaaagctttggagaaggattgccggtacagcaaagggctggtcctcaaggagaagatttttgaggagcagccttgtctccggaaggactctctcagaatgttcctcaaatgtgacatgtcgattcacgatgtttcggtgagtgcagctgagacacaggcgattgtagatgaggccttggggctgcgaaaaaagaggcaagcgctgattgtgcgggagaaggagccggacctgaaacttgtgcagcccattcctttcttcacctggaagtgcctcggagagagcttgctggccatgtacaatcatctcaccacctgtgagcccccacgtcccagccttggcaaaaggattgatttgtcggactaccaggaccccagccagcctcttgagtcctccatggtggtgacgccagttaacgtgatccagccaagcactgtcagcaccaacccagctgtggctgtcgccgagcctgtggtctcctacacctctgtggctacaaccagcttcccactgcacagtcctggtctgttggagacaggcgctcctgtgggtgatatttctgggggagataaatccaagaaaggggtaaaacggaagaagatttcagaagagagtggagaaacagcaaagcggcggtctgcccgtgtccgaaacaccaagtgcaaaaaagaagagaaagtagacttccaggagcttctgatgaagttcttgccgtccaggttaagaaagctggaccctgaggaggaagatgattcctttaataactatgaagtccagtcagaagccaaactggaaagcttcccaagcattgggcctcaaaggctgtcatttgactcagccacattcatggaatctgaaaagcaggacgtgcatgagttcctgctggagaacctaaccaacgggggcatcctggagctgatgatgcgctacctgaaagccatgggccacaagttcttggtaaggtggcctccaggcttggcggaggtcgtgctcagcgtctaccacagctggaggaggcacagcaccagcctgcccaacccgctgctgagggactgcagcaacaagcacatcaaggacatgatgctgatgtctctctcctgcatggaactccagctggaccagtggctgctgaccaaaggcagaagctctgcagtgtctcctcggaactgtcctgctggtatggtgaatggcagatttggacctgacttcccagggacccactgcctgggtgacctcctacagctgtcatttgcctcgtcccagcgcgacctgttcgaggatggttggctggagtttgtggtccgtgtttactggctgaaggctcgcttcctggcgctgcagggagacatggagcaggccctggagaactatgacatctgcacagaaatgctccagagttccaccgccatccaggtggaggcaggggctgaacgaagagacattgtcatccggctgcccaacctccataatgactctgtggtttccctggaggagattgataagaacctgaagtcgctggagcggtgccagtccctggaggagattcagcggctgtatgaagcaggcgactacaaggctgttgtgcatctgctccgccccactttgtgcaccagtgggtttgaccgggccaaacacctggagtttatgacttccattcctgagaggccagcccagctgcttcttctgcaggactccttgctccggctgaaggactatcggcagtgttttgagtgttccgatgtggctctgaacgaggctgtccagcagatggtgaactcaggtgaggctgccgccaaggaggagtgggtggccacagtgacccaactgctgatgggcatcgagcaggccctctctgcggacagcagtggtagcatcctgaaggtatcatcctccaccactggccttgtgcggctcaccaacaacctcatccaggtcattgactgcagcatggctgtgcaggaggaggccaaggagccccacgtctcttcagtgctaccctggatcattctacaccggatcatctggcaggaggaagacaccttccattctctgtgccaccagcagcagctccaaaacccagcggaggaagggatgtcagagacgcccatgctcccatcctccctcatgctgctgaacacagcccacgagtatttgggcagaaggtcctggtgctgcaattcagatggggctctgctgcgattctatgtgcgagtactccagaaggaactggctgcatccacctctgaagacacgcacccttacaaggaggagctggagacagccttggagcagtgcttctactgcctgtacagcttccccagcaagaagagtaaggccaggtacctggaggaacactcggcccagcaggtggatcttatatgggaggatgcactgttcatgtttgagtattttaagcccaagacccttcctgaatttgacagctataagaccagcaccgtgtctgctgacttggccaacctactgaagagaattgccaccattgtgcctcgcacagagaggccagcccttagcctggacaaagtctctgcctacattgagggaacttcaactgaggtaccctgcctcccagagggggctgacccctcccctccagtggtgaacgagctttactacctcctggctgattatcatttcaaaaacaaggagcagtccaaggccatcaagttctacatgcatgacatctgcatctgccccaataggtttgattcctgggcaggcatggctctggcccgggccagccgcattcaggacaagctgaactccaatgagctgaagagtgatgggcccatttggaagcatgccacgcccgtcttgaactgcttccgtcgggccctggagattgacagctccaacttgtccctatggattgagtatggcaccatgtcctatgccttgcactcattcgcctcacgtcaattgaagcagtggagaggcgagctgccccctgagctcgtgcagcagatggagggccggcgcgacagcatgctagagacagccaagcactgtttcacatcagcagcccgctgcgagggtgatggtgacgaggaggagtggctcatccactacatgctgggcaaggtggctgagaagcagcagcagccacccaccgtttacttgctgcactacaggcaggctggccactacctgcacgaggaggctgcccgctaccccaagaagatccactaccacaacccacctgagctggccatggaggccctggaggtgtactttcggctccatgcttccatcctgaagctcctggggaagcccgattctggggttggtgcagaggtcctggtcaactttatgaaggaggctgcagaaggaccctttgccaggggcgaggagaagaacacacccaaagcttcagaaaaggagaaggcctgcctggtggacgaggactcccactcttcagctgggacactgccgggccccggagcctccctcccctcctcctctggcccaggtctgacatccccaccttacacagccactccgattgaccacgattacgtcaaatgtaaaaaaccccaccagcaggcaacgccggacgaccgaagccaggacagcacagccgtagcactctcagactctagctcaacgcaggacttctttaatgagcccaccagcttactggaaggctccaggaaatcctacacagagaagaggctgcccattctcagttcccaagcaggagcgacgggtaaagatcttcagggggccacagaagaaagaggaaaaaacgaggagtcattggagagtacagaaggcttccgggctgcagagcaaggtgtccagaagcctgctgcagaaaccccagcctctgcttgcatccctggcaagccctcagcatccacacccaccctgtgggatgggaagaagagaggggacctcccaggggagccagtggccttcccccaggggctgccggctggtgctgaggagcagcggcagtttctcacagagcagtgcatcgcctccttccgcctgtgcctgagccgcttcccccagcactataagagtctctaccgtctggccttcctctacacctacagcaagacccaccggaacctccagtgggcccgcgacgtgttgctaggcagcagtatcccgtggcaacaactgcagcacatgccggcacaggggctcttctgcgagaggaacaagaccaatttcttcaacggcatctggcggatccccgtggacgagattgaccggccgggcagctttgcctggcacatgaaccgctccatcgtgctgctgctcaaggtgctggcccagctgcgggaccacagcaccctgctgaaggtgtcctccatgcttcagcggaccccagaccagggcaagaagtatctgcgagatgctgaccgccaggtcctggcgcagcgggccttcatcctcactgtgaaggtgctcgaagacacgctgagcgagctcgcagaggggtcagaacgcccagggcccaaggtctgtggcctccccggagccaggatgaccaccgatgtctcacacaaggccagtcctgaggatggccaggagggcctcccccagccgaagaagccccctctggctgatggctcagggccagggcccgagccaggaggcaaagtgggcctcctcaaccaccggcctgtggccatggatgcaggagacagtgcagaccaaagcggggagcggaaggataaagagagcccacgggcagggcccactgagcccatggacacgagtgaggccactgtttgccactcagacttggagcggacaccacccctgctgccaggtcgccccgcaagggaccggggccccgagagccggcccactgagctgtccctggaggagctgagcatcagtgcccggcagcagcccaccccgctcaccccagcccagccagcccccgcccccgcccccgccaccaccacagggaccagggcagggggccacccggaggagccgctctcccggctcagccgcaagaggaagctcctggaggacacagagtcaggcaagacacttctgttggatgcctaccgtgtgtggcagcagggccagaagggtgtggcctatgacctgggccgtgtggagaggatcatgtcggagacctacatgctcatcaagcaggtggatgaggaggctgcgctggagcaggctgtgaagttctgccaggtccatcttggggctgccgcccagagacaggcctcgggggacacccccaccactccaaagcaccccaaagacagccgagagaacttctttcctgtgacagtggtgcccacagcccctgaccctgtgccagctgactctgtccagcggcccagtgatgctcacaccaagcctcgccctgcactagctgccgccacaactattatcacctgccctccgtcagcatcagcttccaccctggaccagtccaaggaccctgggcctccccggccacacaggcctgaagctacccccagcatggcctctctgggcccagagggagaagagctggcgagagtggcagagggcaccagcttcccgcctcaggagccacggcacagtccgcaggtgaagatggcccccacaagttccccggcagagccacactgctggccggcagaggctgccctgggcacaggcgctgagcccacctgcagccaggaggggaaactgaggcctgagccgagaagggatggggaggctcaggaggctgcgagtgagactcagcccctgagctctcccccaacagctgccagctccaaggcccccagcagtgggagtgcccagccaccagagggtcacccaggcaagcctgagcccagccgggctaagtcccgccccctgcccaacatgccaaagctggtcatcccctccgccgccaccaagttcccccctgagatcaccgtcacgccacccaccccaaccctgctctcccccaaaggcagcatctcggaggagaccaagcagaagctgaagtcagccatcctttctgcccagtctgctgccaacgtgaggaaggagagcctatgccagccagccctggaggtcctggagacatccagccaggagtcctcgctggagagcgagacagacgaggacgacgactacatggacatttgaggggccactgcagccccaccgccacgccccaggggaccagccaggcctggaatgccccctgggcaggaccctgggcaggaccagaggcccacatggatgccactccccacacagcccccaggcctgcccagcccacctcctcatggcatcctccctgtacccaggtcaggctgtccacaccacatgggagcccagaggaggaggggcccgccttagccatgtgaaggtggattggtcgccatctgcacgccaggcggcatccttttctatgaagtgttgactttgtaaatctgcccacacccagctggccatatccacccctcgacgccgggatgagccggctctgcctgtgtcacagtggaggggtcctttagggccaggctcacccctcaccctttttttggttgcttttctaataaagatggaacagtt

SEQ ID NO: 13 - DNA, 1620 bp, Homo sapiens
  gene (1)...(1620): LGALS9; lectin, galactoside-binding, soluble, 9 (galectin 9)
caaaggacttcctagtgggtgtgaaaggcagcggtggccacagaggcggcggagagatggccttcagcggttcccaggctccctacctgagtccagctgtccccttttctgggactattcaaggaggtctccaggacggacttcagatcactgtcaatgggaccgttctcagctccagtggaaccaggtttgctgtgaactttcagactggcttcagtggaaatgacattgccttccacttcaaccctcggtttgaagatggagggtacgtggtgtgcaacacgaggcagaacggaagctgggggcccgaggagaggaggacacacatgcctttccagaaggggatgccctttgacctctgcttcctggtgcagagctcagatttcaaggtgatggtgaacgggatcctcttcgtgcagtacttccaccgcgtgcccttccaccgtgtggacaccatcttcgtcaatggctctgtgcagctgtcctacatcagcttccagcctcccggcgtgtggcctgccaacccggctcccattacccagacagtcatccacacagtgcagagcgcccctggacagatgttctctactcccgccatcccacctatgatgtacccccaccccgcctatccgatgcctttcatcaccaccattctgggagggctgtacccatccaagtccatcctcctgtcaggcactgtcctgcccagtgctcagaggttccacatcaacctgtgctctgggaaccacatcgccttccacctgaacctccgttttgatgagaatgctgtggtccgcaacacccagatcgacaactcctgggggtctgaggagcgaagtctgccccgaaaaatgcccttcgtccgtggccagagcttctcagtgtggatcttgtgtggagctcactgcctcaaggtggccgtggatggtcagcacctgtttgaatactaccatcgcctgaggaacctgcccaccatcaacagactggaagtggggggcgacatccagctgacccatgtgcagacataggcggcttcctggccctggggccgggggctggggtgtggggcagtctgggtcctctcatcatccccacttcccaggcccagcctttccaaccctgcctgggatctgggctttaatgcagaggccatgtccttgtctggtcctgcttctggctacagccaccctggaacggagaaggcagctgacggggattgccttcctcagccgcagcagcacctggggctccagctgctggaatcctaccatcccaggaggcaggcacagccagggagaggggaggagtgggcagtgaagatgaagccccatgctcagtcccctcccatcccccacgcagctccaccccagtcccaagccaccagctgtctgctcctggtgggaggtggcctcctcagcccctcctctctgacctttaacctcactctcaccttgcaccgtgcaccaacccttcacccctcctggaaagcaggcctgatggcttcccactggcctccaccacctgaccagagtgttctcttcagaggactggctcctttcccagtgtccttaaaataaagaaatgaaaatgcttgttggcaaaaaaaaaaaaaaaaaaaaa

SEQ ID NO: 14 - DNA, 2385 bp, Homo sapiens
  gene (1)...(2385): CEBPA; CCAAT/enhancer binding protein (C/EBP), alpha
atggagtcggccgacttctacgaggcggagccgcggcccccgatgagcagccacctgcagagccccccgcacgcgcccagcagcgccgccttcggctttccccggggcgcgggccccgcgcagcctcccgccccacctgccgccccggagccgctgggcggcatctgcgagcacgagacgtccatcgacatcagcgcctacatcgacccggccgccttcaacgacgagttcctggccgacctgttccagcacagccggcagcaggagaaggccaaggcggccgtgggccccacgggcggcggcggcggcggcgactttgactacccgggcgcgcccgcgggccccggcggcgccgtcatgcccgggggagcgcacgggcccccgcccggctacggctgcgcggccgccggctacctggacggcaggctggagcccctgtacgagcgcgtcggggcgccggcgctgcggccgctggtgatcaagcaggagccccgcgaggaggatgaagccaagcagctggcgctggccggcctcttcccttaccagccgccgccgccgccgccgccctcgcacccgcacccgcacccgccgcccgcgcacctggccgccccgcacctgcagttccagatcgcgcactgcggccagaccaccatgcacctgcagcccggtcaccccacgccgccgcccacgcccgtgcccagcccgcaccccgcgcccgcgctcggtgccgccggcctgccgggccctggcagcgcgctcaaggggctgggcgccgcgcaccccgacctccgcgcgagtggcggcagcggcgcgggcaaggccaagaagtcggtggacaagaacagcaacgagtaccgggtgcggcgcgagcgcaacaacatcgcggtgcgcaagagccgcgacaaggccaagcagcgcaacgtggagacgcagcagaaggtgctggagctgaccagtgacaatgaccgcctgcgcaagcgggtggaacagctgagccgcgaactggacacgctgcggggcatcttccgccagctgccagagagctccttggtcaaggccatgggcaactgcgcgtgaggcgcgcggctgtgggaccgccctgggccagcctccggcggggacccagggagtggtttggggtcgccggatctcgaggcttgcccgagccgtgcgagccaggactaggagattccggtgcctcctgaaagcctggcctgctccgcgtgtcccctcccttcctctgcgccggacttggtgcgtctaagatgagggggccaggcggtggcttctccctgcgaggaggggagaattcttggggctgagctgggagcccggcaactctagtatttaggataaccttgtgccttggaaatgcaaactcaccgctccaatgcctactgagtagggggagcaaatcgtgccttgtcattttatttggaggtttcctgcctccttcccgaggctacagcagacccccatgagagaaggaggggagcaggcccgtggcaggaggagggctcagggagctgagatcccgacaagcccgccagccccagccgctcctccacgcctgtccttagaaaggggtggaaacatagggacttggggcttggaacctaaggttgttcccctagttctacatgaaggtggagggtctctagttccacgcctctcccacctccctccgcacacaccccaccccagcctgctataggctgggcttccccttggggcggaactcactgcgatgggggtcaccaggtgaccagtgggagcccccaccccgagtcacaccagaaagctaggtcgtgggtcagctctgaggatgtatacccctggtgggagagggagacctagagatctggctgtggggcgggcatggggggtgaagggccactgggaccctcagccttgtttgtactgtatgccttcagcattgcctaggaacacgaagcacgatcagtccatcccagagggaccggagttatgacaagctttccaaatattttgctttatcagccgatatcaacacttgtatctggcctctgtgccccagcagtgccttgtgcaatgtgaatgtgcgcgtctctgctaaaccaccattttatttggtttttgttttgttttggttttgctcggatacttgccaaaatgagactctccgtcggcagctgggggaagggtctgagactccctttccttttggttttgggattacttttgatcctgggggaccaatgaggtgaggggggttctcctttgccctcagctttccccagcccctccggcctgggctgcccacaaggcttgtcccccagaggccctggctcctggtcgggaagggaggtggcctcccgccaacgcatcactggggctgggagcagg

SEQ ID NO: 15 - DNA, 4648 bp, Homo sapiens
  gene (1)...(4648): LRRN4; leucine rich repeat neuronal 4
gggggtgggggaaggagggacggccggtcccgtcagtcaggcagcgggagccgccgggagcggatggcggcggccgtagcggctccactcgccgccgggggtgaggaggcggcagccacgacctccgtgcccgggtctccaggtctgccggggagacgcagtgcagagcgggccctagaggaggccgtggccaccgggaccctgaacctgtctaaccggcgcttgaagcacttcccccggggcgcggcccgtagctacgacctgtcagacatcacccaggctgacctgtcccggaaccggtttcccgaggtgcccgaggcggcgtgccagctggtgtccctggagggcctgagcctctaccacaattgcctgagatgcctgaacccagccttggggaatctcacagccctcacctacctcaacctcagccgaaaccagctgtcgctgctgccaccctacatctgccagctgcccctgagggtcctcatcgtcagcaacaacaagctgggagccctgccccctgacatcggcaccctgggaagcctgcgacagcttgacgtgagcagcaacgagctccaatccctgccctcggaactgtgtggcctctcttccctgcgggacctcaatgtccggaggaaccagctcagtacgctgcccgaagagctgggggacctccctctggtccgcctggatttctcctgtaaccgcgtctcccgaatcccagtctccttctgccgcctgaggcacctgcaggtcattctgctggacagcaaccctctgcagagtccacctgcccaggtctgcctgaaggggaaacttcacatcttcaagtatttgtccacagaggccgggcagcgtgggtcggccctgggggacctggccccttctcggcccccgagtttcagtccctgccctgcagaggatctatttccgggacatcggtacgatggtgggctggactcaggcttccacagcgttgatagtggcagcaagaggtggtctggaaatgagtcaacagatgaattttcagagctgtcattccggatctcagagctggcccgggagccccggggacccagagaacgcaaggaggatggctcagcggacggagaccctgtgcagattgacttcatcgacagccatgtccccggggaggatgaagagcgaggcactgtggaggagcagcgaccacccgaattaagccctggggcaggggacagggagagggcaccaagcagcaggcgggaggagccggcaggggaggagcggcggcgcccggacaccttgcagctgtggcaggagcgggaacggcggcagcagcagcagagcggggcgtggggggccccgaggaaggatagcctcttgaagccagggctcagggctgttgtgggaggggccgccgccgtgtccactcaagccatgcacaacggctcgcctaagtccagtgcctcccaagcaggggctgcagcggggcagggagcccccgcccctgcccctgcctcccaagagccccttcccatagctggaccagcgacagcacctgctccacggccacttggctccattcagagaccaaacagcttcctcttccgttcctcctctcagagtggctcaggcccttcctcaccagactctgtcctgagacctcggcggtacccccaggttccagatgagaaggacttaatgactcagctgcgccaggtccttgagtcccggctgcagcggcccctgcctgaggacctggccgaggctctggccagtggggtcatcctgtgccagctggccaaccagctacggccgcgctccgtgcccttcatccatgtgccctcccctgctgtgccaaaactcagtgccctcaaggctcggaagaatgtggagagttttctagaagcctgtcgaaaaatgggggtgcctgaggctgacctgtgctcgccctcggatctcctccagggcactgcccgggggctgcggaccgcgctggaggccgtgaagcgggtggggggcaaggccctaccgcccctctggcccccctctggtctgggcggcttcgtcgtcttctacgtggtcctcatgctgctgctctatgtcacctacactcggctcctgggttcctaggccccaaaatcggccctccctcacccctttcccttcctctctatttataaggtccctgctccacccgaccccacctgcggtgccttcagccccaaccaaagacactagtgcacccccttcacagacactgacctcagaggccccactctggtgcccccagaccctgggcccccagcctctggcctccctccagtagccccacgagtccccacctctcagtgctgacggtgccttcatgtccccgccggccctgcccctgccctctgtaccccgtgaggggtggcaggagctggagtctcccccttcctcctgtgccctccccttccccccccaacagctgctatgggggggctaaattatctctattttgtagagaggatctatatttgtaggggttcggggcccaggccgggtccctatctctgtgtataaactgtacagaccgtggccgccctgcctgtgtgtgtgtgtgtgtgtgtgtgtgtgtgtgtgtgtgtgcgcgcgcgcgcgcgtctgctccgcgtgttggtggctgtggccatggctctgtgcccaccagcatctccctcctgagatgccggcctctcatgctcccggagcgtccgccaaccccccgtgtcacctcccttctgttatcgctgacagctttcttgcgtctcatttgtcgccgagccccgagcgcacggtgatgctcgggtctgcccccgaccccctgccacaggccggaagccgcagggggcaccgtggggaagctaacccggccccttcccccaggagtcactgtgccagccccaccacatcctggaagaggagggggccccgggaaggggcctcccctacatcgctgctgtcgtccacgccctgctggaccggccttaggggtgaaggtgggggcacagggcccaccccgcccgggaccccgggagcagaagacgcgcctgggctccgcgctctcagagaagcacgtggtgagggtggcctgggcctgggcacccttggcctgccggcctggctgcctctggggtcgagggtctgggtggaaggaccgcgggtgtcgcaggtgtcgtgtcagccgcaataaacagaagccagaagccctctgggtggccctcaggtgtaactgtgtctgagagcacgtgcgtcgcggggttgttgggggggcctctgtttccggaattcaccgcccagtcgccagggggcgcccgcgggagcccggcgggggcacgcgccacgtggccgggagacgcacagggcgccagcgtcgccgcgaggcctctggggtgggttcgccaggagggcacgttcgcgccgcgccgcggagggaggctgagagggccgggccgggcggcggggacacggcggtaccgtcctgcgctcgggaacgcggaatgttgccctggtcgccgccgcggtggggcgcgggcccggaggaggcgcccgagggaccaggccttccggcgggcaaggaccagggcgagccctggagcgccggagcggacgcccccagggagctggggacacccccatgggacaggtgaggggctcggacggagccgtggggggacagcgactgggggcctccatgccttcaccacgcgctctacggaggcttctccattgtccctgctggagattgggtccctcggtctcccttgcaccaggcacccttgggctccaggggagggtgcgtgggggcggtgtggctgcagcctcagtccctgcccctccagcccacagcccccatcccgtagcccttcctggatctggagagagcagctgctgctgccccaccacctgccaggcccggccactgagcagccccccggagccccagatccccgttccccccaggtggcctgggaggtggccccctcgaggatgactccactagcgccctgggaccccaagtatgaagccaaagcaggacctcggccggtgtggggggccaactgtagctcaggagcctcgttctcaggccggacgctgtgtcacccctcattctggccgctgtatgaagcagcctcgggcaggggtctcaggcccgtggcccctgccacagggcactggaatggacagcaggcgcccccagatgcagggttcccggtggtgtgctgtgaagatgtcttcctctcggaccctctgctgccccgggggcagcgtgttcccctgtacctgtccaaggccccccagcagatgatgggctccctgaaactgctgccgccgccccccatcatgtctgccagggtgctcccccgcccatcaccctcccggggcccctccactgcctggctcagcgggccggagctgatcgctctcactggcctgctgcagatgagccagggggagcctaggcccagctcctccgcggttggccccccagaccatacctctgacccacccagcccctgtggtagccccagcagttctcagggtgctgacctctctctcccacagaccccagacacccattgtccatagccttctcagggcagagtgggctggttgtgttgacaataaaacagtgttggtttgc

SEQ ID NO: 16 - DNA, 1815 bp, Homo sapiens
  gene (1)...(1815): STXBP2; syntaxin binding protein 2
gcggccgctcgcccctcggggaatatggcgccctcggggctgaaggcggtggtgggggaaaaaattctgagcggagttattcggagtgtcaagaaggatggggagtggaaggtgcttatcatggatcacccaagcatgcgcatcttgtcttcctgctgcaaaatgtcagatatcctggctgagggcatcaccattgttgaagacatcaacaaacggcgggaacccattcccagtctggaggccatttatttgctgagccccacggagaagtcggttcaggccctgatcaaagacttccaggggaccccgactttcacctacaaagcggcccatatcttcttcaccgacacctgccccgagcccctgttcagtgagctaggccgctctcgtctggcaaaggtggtgaagacgttgaaggagattcaccttgccttcctcccctacgaggcccaggtgttctccctcgatgctccccacagcacctacaacctctactgccccttccgggcagaggagcgcacgcggcagctcgaggtgctggcccagcagattgccacgctgtgcgccaccctgcaggagtacccggccatccgctaccgcaagggcccagaggacacagcccagttggcccacgccgtcctggccaagctgaacgccttcaaggcagacactcccagtctgggcgagggcccagagaaaacccgctcccagctgctgataatggaccgggcagctgaccccgtgtccccactactgcatgagctcacgttccaggccatggcgtatgatctgctggacatagagcaggacacatacaggtatgagaccaccgggctgagcgaggcgcgggagaaggccgtcttgctggacgaggacgatgacttgtgggtggagcttcgccacatgcatatcgcagatgtgtccaagaaggtcacggagctcctgaggaccttctgtgagagcaagaggctgaccacggacaaggcgaacatcaaagacctatcccagatcctgaaaaagatgccgcagtaccagaaggagctgaataagtattctacgcacctgcatctagcagatgattgtatgaagcacttcaagggctcggtggagaagctgtgtagtgtggagcaggacctggccatgggctccgacgcagagggggagaagatcaaggactccatgaagctgatcgttccggtgctgctggacgcggcggtgcccgcctacgacaagatccgggtcctgctgctctacatcctccttcggaatggtgtgagtgaggagaacctggccaagctgatccagcatgccaatgtacaggcgcacagcagcctcatccgtaacctggagcagctgggaggcactgtcaccaaccccgggggctcggggacctccagccggctggagccgagagaacgcatggagcccacctatcagctgtcccgctggaccccggtcatcaaggatgtaatggaggacgccgtggaggaccggctggacaggaacctgtggcccttcgtatccgaccccgcccccacggccagctcccaggccgctgtcagtgcccgcttcggtcactggcacaagaacaaggctggcgtagaagcccgggcgggcccccggctcatcgtgtatgtcatgggcggtgtggccatgtcagagatgagggccgcctacgaggtgaccagggccaccgagggcaagtgggaggtgctcattggctcctcacacatcctcaccccgacccgcttcctggatgacctgaaggcactggacaagaagctggaggacattgccctgccctgacgcggccgc

SEQ ID NO: 17 - DNA, 7300 bp, Homo sapiens
  gene (1)...(7300): SH3BP2; SH3-domain binding protein 2
cagccgggtgacccaggccgaggccggcagaagacagcctgatgccttgaagacttcctcttgcacttttgttggagggtgctggtttgctaaaagcagagagtatttttctttttatttattttgtttttaattttttaattttagctccagctcagttgcccagactggagagcagtggccaatcatagcttactgcctcctggaactcctggctcaatcgatcctcctggataagcctcctccgggtactatagcttcatggcggctgaagagatgcattggcctgtccctatgaaggccattggtgcccagaacctgctaaccatgcctgggggcgtggccaaggctggctacctgcacaagaagggcggtacccagctgcagctgctgaaatggcccctgcgctttgtcatcatccacaaacgctgcgtctactacttcaagagtagcacctctgcctccccgcagggcgccttctccctgagtggctataaccgggtgatgcgggcggctgaggagaccacgtccaacaacgttttccccttcaagatcatccacatcagcaagaagcaccgcacgtggttcttctcggcctcctccgaggaggagcgcaagagctggatggccttgctgcgcagggagattggccacttccacgaaaagaaagacctgcccttggacaccagcgactccagctcggacacagacagcttctacggcgcagttgagcggcctgtggatatcagcctttccccgtaccccacggacaatgaagactatgagcacgacgatgaggatgactcctacctggagcctgactccccggagcccggaaggcttgaggatgccctgatgcacccaccggcttacccaccacccccagtgcccacgcccaggaagccagccttctctgacatgccccgggcccactcctttacctccaagggccccggtcccctactgccacccccgccccctaagcacggcctcccagatgttggcctggctgctgaggactccaagagggacccactgtgcccgaggcgggctgagccttgccccagggtacctgctaccccccgaaggatgagcgatccccctctgagcaccatgcccaccgcacccggcctccggaaacccccttgcttccgggagagtgccagccccagcccggagccctggacccctggccacggggcctgctccacttccagtgctgccatcatggccactgccacctccagaaactgtgacaaactcaagtccttccacctgtccccccgaggaccacccacatctgagcccccacctgtgccagccaacaagcccaagttcctgaagatagctgaagaggaccccccaagggaggcagccatgcccggactctttgtgccccccgtggctccccggcctcctgcgctgaagctgccagtgcctgaggccatggcgcggcccgcagtcctgcccaggccagagaagccgcagctcccgcacctccagcgatcacccctcgatgggcagagtttcaggagcttctcctttgaaaagccccggcaaccctcacaggctgacactggcggggacgactcggacgaggactatgagaaggtgccactgcccaactcggtcttcgtcaacaccacggagtcctgcgaagtggaaaggttgttcaaggctacaagcccccggggagagccccaggatggactctactgcatccggaactcctctaccaagtcggggaaggtcctggttgtgtgggacgaaacctctaacaaagtgaggaactatcgcatttttgagaaggactctaagttctacctggagggcgaggtcctgtttgtgagtgtgggcagcatggtggagcactaccacacccacgtgctgcccagccaccagagcctgctgctgcggcacccctacggctacactgggcctaggtgatggcagtccatgtggctgccaggccaaggcagtcacaggggccctgaccccaggccacacagacggacatgggcccacatgggagggtgagcaggagcaaggctgtgcttgcctagggcctctgtgatggacatctcgtaggacccagccagtctcatccagcaggttgggttctagggctgaaccaggcgccaggctccagaggacgaagggactctgttgccccacactaacttgccctgtcccaatcccagaaacccaggaccaagctgtgcctgggctccaaggacaggaacactggtccccccatcacactcacccctaagtgggctgggagccaggcagggccagggcagctgggtgggggccggggctggccctgggacccccaggaacgctaagacacaggctccagtaggggctgttgcctccaataaagcagcagtgagctttgccttggtggctggggcttgattgggaaggaggggattaccagcttactgggtgcccatgctgatgtctaagtggtgaccgcagcagtacccgggaaccccaacagttggttgtcttgtcttccagggtgcaggtcactgagtgacttccccagggtgcacagcgagtaacagatcaggacccaaacttgggcagtctgggctgggagcccacaccccactcaccagttctgctgcctcaggtcaggccagggcagtgctgctgcagagctagaaggccctgcagctacagctgcttcattccctgcattagtgcctggttactgggtacctcctgagtggctgtccccgttccagaacttgcatacactgagcgggctacagagctagaagccctgcagctacagctgcttcattccctgcattagcgagcagttattgggtacctcctgcatgcctggtcccattccagacaggggcctctggcctggctgagttcacagcccagtctggggacagctgggtatgaggtgcttacggcacagtgtccagggcagctgggtgtgcagggactgggggctcccggaagattttttggaggaagtaacagctacgatgggatgggaacagtggaccctaagcaggccaagggtgcgtagggacggtggtacccagatgcccaagtcttccaggcaatacctggctcaggcccagccccaatccatccccttactttctgccatggagttccagcaggtcactctccctggcacaccttccaggctggatttttaatgaaacagactcagggaggtaggggctggcagggaccctagaatccttgtgatttttcttagcaccttatgtcagggaaacctaaactgaggtcagcacttgggcccactgacagtgactgactgggggagaaggtcctgcagcccccttcccctgggtgtgttctggggacctgtggtttgctggcggaaacaaatgatgaggctggttagcggatgtgggaggctgtgaccccagggggccatagggtgcggtggaactgcaggccctgcagatgacggcagccagctgcttccaggaaccaggtgtccaaggccacctctgcaggggtttcctcttcagcctgcctggggtgagaggtcagtgcaccacagccgaggctggagcacagggagcttctgttgttctgatctatctctggaaaaccagccattcctcctccctgcagtcagaattctttgccctgtctgacctgaacttgcttagggagtcatgccactccccactgtggccatagtttctcttcctgtaaaattttattattttagttttttgtttttgagatgtagtctcaccctgtcgcccaggctggagtgcaatgccgtgatctccgctcactgccacctccgcctctctagttcaagcgattttcctgcctcagcctcccgagtagctgggattccaggcgcccgccaccacgcctggctaattttttgtatttttagtagagacgggattttatcatgttggccaggctggtctcgaactcctgacctcaggtgatctgcccaccttggcctcccaaagtgctgggattacaggcatgagccactgtgcctggccccttcctgtaaaatttttaaatggagaattgggtgcgagatgtggtttccagcctggtgcctggggtgctgagctagtgagtggtgcagtccaggacacctttgctttatgtcacttacacggtcacctggagccggctcaagtggctaaagcatcctggggcccagagccaggtgatagtccctctggccaactggacagttgaggcttgtggttaacccgaagcccagctggggccttggtccagcttcgcttcccagattctgcacctgctagcacagctgtccacgtctgtgtgagctgttctaggccgagggcctcagtttcaagagtgtgttggggtgggatggggcaggccgtggtcctccagcatgaagaaggagccatgaggagttcccatgacctcccgagacttgccataagtgttctagtccacatataagggtagggttgggattaccatttactgaccacatctgtgaggtgccgagctgggtgcttgacatcatttgcttggagaagcagctgttagtagacccattttacaggtgagagaaccaagtctcacagaggcctgggttcaagtcccacctctgccactaactggcatgtgaccctatctatccttcactgctctgagcctagaccctggcccctgcctggctccctgccaggctccctgccacccctcacgacctctgatggtcgttgtgggggtctcttgcctggctcccagggctagggttagggctctggaggtgctttcactcaaccaagggggccacagcactggggagtgaaactgccccgcctcaccctgcgttgccctctgggtctgtgagggtgggctggcaggaggcctaggccttgccctaggggcagtcctgcttcctcattttatagatagggaaactgaggctttgggaggactcactgacatacctaccttcaagatgagttcaggtgggctcagttctggggcttgggaaaagggccccagtggctttgggaagcacccccagcccagggtgaaacatgcttcttctcttcctgtggttccatccgaaggattgtggtgagccccgtgccttcagttaataaagatttgtattgtgaaaagattttttcttttttttttgggacacagtctcactctgtcgcccaggctagagtggattggcgtgatctcggctcaatgcaaatctccagggttcaatcgattctcctgcctcaccctcccatgtagctgggattacagctgcctgccaaatttttgtatttttagtggaaccggggtttcaccatgttggccaggctggtcttgaactcctgacctcaactgatccgcccaccttggcctcccaagtgctgggattacaggcgcgagccacggcgcccagccttgaaaagatgtttttagaaccagaagaaacctcggttcccactgatccttctgggccacgttgtgcggagctcccctgctggttggggctcagcgcagccccagggaggtgcttcctgcacctcaggatgggcgagggtgggcattgggggagagggggacctgggacctgcggcttagttccctgaggcaggcagggcttattggggccatttcatagaaaggcagattgaagctcagcagggaagaagcttttgagggtgatccaggcgctggagggatggcctaggacaccagggtcacaccaggaacatgggagggccgtgcttgtctctagacgaggggaatgggggaagggccacaacctctgtttctgtgacccagcagcatcaagcccctcgctgggcacctcgcacacaccccctgccttatctctgcctgcacgccctgttccctccacctagactgcctgctgagggggcagtgccaggaggttgcctgtccttggggaagaggggcagtgaccctgtgaagatgcttgacagacaacccccaccacctcagaagtgtgtgtgagtggtgaacccttttaagccatcttccagccattctcactggagggagatttgatgggtacagagcagacccctacctgtctaccctccttcggacccctaggaagcttcgcaggccttccaggctgccagacagctgccctggcgttgccgtctgcttcttccctggccccactctgaggggctcagagctgaggcagaatccctttttcattcatttcctgcagaataaaacaacatacagaaaagtgaataaaacataaatgcacaacctaacacactgttaggaagtaaacgatctgcaaccaccatcaggaaatagttttgccagcacccaagtgccctcccctcacagtgtcacttccggcctctctgccctggcttatgtgagtcttgtgttcttgtttttctaaaaagtcttcagcacccaattatgcaagcattgcagtattttcctgtttctgtgctttatccccttgaatcatacagatgcaaattctggcagctggcttctttggctcgttattatgtctgtgagatttattcatgttgctgtgcgtagtatagtttgtgcatgttcattgctaaaaacttccattgtttggctgtatcgtagttcacagattcatttcactgtcagtcaagcttgtccaatgcatgcagcccaggatgcctttgaatgtggcccaacacaaatttgtaaactttcttaaaacattataaagatttttgtttgcgatttttttttttagctcatcagctatagttagtggtagtgtattttatgcgtgacccgagacagttcttccggtatggtccatggaagccaaaagattggacatgcctgctgtagatggacagttggtttgtttctagtttggggtaactacacacaatgctgctagcaacagttttgtccatgtctctgatgcacgtgtgttttttgcaaatggtgcacaaatttttctagggtttgtactcaggagtctgactcctgggttctagggtatgaagatctttctaaatattgttctagtttacgtgcccaccagcagtaaaacagaattcccttgccttcccatccttggcagacatttcacttttgccagtctggtggggtgtatagttatggccttaatttgcatttagctaattaccaaggagattgagcatatttttatgtttttattaaccattttgattttgtctcctgtgaagtgtctatcatcttttgcccattttttaacttgttgtctttttctttttcttttctttttttttttttt

SEQ ID NO: 18 - DNA, 1692 bp, Homo sapiens
  gene (1)...(1692): RNF24; ring finger protein 24
ctggggtttgggggaccgagccgctccgggttcgggatgctgaggagaccgcgggcccgcgccagcctcccacacacacacactctcccggcgcctcggccggccgggcgcggtgtgcaggggtgaggcggtgtctccaggctgaggagggacgcggaggagggtccggcgcggccccgggactggagggtgagtccctcacaaagggcggagcggggctgtcacctggagatcacggagggaagttcatccatgagctcggatttcccacattacaacttcaggatgcctaatattggattccagaatctgcctctcaacatatatattgtggtttttggtactgctatatttgtcttcatccttagtttactcttctgttgctacttgattaggctaagacatcaagcacacaaagaattttatgcctacaaacaggttatattaaaagagaaagtaaaagaattgaatttacatgagctctgtgcagtgtgcctagaagacttcaagcctcgagatgagttggggatttgcccatgtaagcacgccttccacagaaagtgccttattaagtggctggaggttcgtaaagtgtgtcccctgtgcaacatgccagttctacagctggcccagttgcacagtaagcaggaccgtggaccccctcaggggccccttcctggggcagagaacattgtatagcttaccgcaaggatcagactgttgctggacacgacgtctgtgtggagccaggaggaacacatgtggtgtctgtatggctgctctctacctaggacaccagctgccacttcttttgcctcatgaagaactcttgggccagccaaactgggaacctaggtgtctgggtcttgtgacaaccaaagcactttgacactaccccctgccgagagaagaggagtggatgagcctgcgggtttgcctcaagaaacttcatgagggtctcttactaactccattacactctctctcctggagcctcatctccatgtcaagcaggagggtaaagaagggaactaagagcaggtctttcaagccacacccccacctgcggatggatgggtttctctgtaggccatgcaggcctttgtcgcagcaaaccttcccagcagcccttgagccaagtaaaaccagcacaaccagccaccagtggttggtgaggcagtgcccacaaggctcatgttgtatgcctttgataaggccatcttggctttgagtagcagtgttcctcgtcacccatttccccctcaggattacaacacctgctatcaaatcatctaagctgaaaacatgagatgcgcttggaaaggcctagtcagaagccatttcctcttatcatttccctctcctatgcaccagtaaggcccgtccagagccccagcagggagtgggccctgagtccacactgtccctgagtgatccaggaggctgcccacatccccacatgtgcactgtggttccagtgtagctgctgtgagcccactgccactgcctcagaagggagccactgtgaacctcttggctccaaagagcagtggctttttgagaatggcccatacttctggcccggctggatgaagggaatgccgaccctttggcctctccccctccttccatctcttctctctcttgccctatctctctttctacttcagaaaaaaaaaaaaaaaaaaaaaa

SEQ ID NO: 19 - DNA, 1743 bp, Homo sapiens
  gene (1)...(1743): PLAUR; plasminogen activator, urokinase receptor
cagtatccctcctgacaaaactaacaaaaatcctgttagccaaataatcagccacattcatatttaccgtcaaagtttttatcctcattttacagcagtggagagcgattgccccgggtcccacgttaggaagagagagaactgggatttgcacccaggcaatctggggacagagctgtgatcacaactccatgagtcagggccgagccagccccttcaccaccagccggccgcgccccgggaaggaagtttgtggcggaggaggttcgtacgggaggagggggaggcgcccacgcatctggggctgactcgctctttcgcaaaacgtctgggaggagtccctggggccacaaaactgcctccttcctgaggccagaaggagagaagacgtgcagggaccccgcgcacaggagctgccctcgcgacatgggtcacccgccgctgctgccgctgctgctgctgctccacacctgcgtcccagcctcttggggcctgcggtgcatgcagtgtaagaccaacggggattgccgtgtggaagagtgcgccctgggacaggacctctgcaggaccacgatcgtgcgcttgtgggaagaaggagaagagctggagctggtggagaaaagctgtacccactcagagaagaccaacaggaccctgagctatcggactggcttgaagatcaccagccttaccgaggttgtgtgtgggttagacttgtgcaaccagggcaactctggccgggctgtcacctattcccgaagccgttacctcgaatgcatttcctgtggctcatcagacatgagctgtgagaggggccggcaccagagcctgcagtgccgcagccctgaagaacagtgcctggatgtggtgacccactggatccaggaaggtgaagaagggcgtccaaaggatgaccgccacctccgtggctgtggctaccttcccggctgcccgggctccaatggtttccacaacaacgacaccttccacttcctgaaatgctgcaacaccaccaaatgcaacgagggcccaatcctggagcttgaaaatctgccgcagaatggccgccagtgttacagctgcaaggggaacagcacccatggatgctcctctgaagagactttcctcattgactgccgaggccccatgaatcaatgtctggtagccaccggcactcacgaaccgaaaaaccaaagctatatggtaagaggctgtgcaaccgcctcaatgtgccaacatgcccacctgggtgacgccttcagcatgaaccacattgatgtctcctgctgtactaaaagtggctgtaaccacccagacctggatgtccagtaccgcagtggggctgctcctcagcctggccctgcccatctcagcctcaccatcaccctgctaatgactgccagactgtggggaggcactctcctctggacctaaacctgaaatccccctctctgccctggctggatccgggggacccctttgcccttccctcggctcccagccctacagacttgctgtgtgacctcaggccagtgtgccgacctctctgggcctcagttttcccagctatgaaaacagctatctcacaaagttgtgtgaagcagaagagaaaagctggaggaaggccgtgggcaatgggagagctcttgttattattaatattgttgccgctgttgtgttgttgttattaattaatattcatattatttattttatacttacataaagattttgtaccagtgg

SEQ ID NO: 20 - DNA, 1797 bp, Homo sapiens
  gene (1)...(1797): RIS1; Ras-induced senescence 1
ccggcgccgggggcggcggggaaccccaaacgcaaccgggtctggagggatccccgcgccgagccagccgccgtcaccgcctccgcgccgcccctgcgggcttggcaggcgcccggcgcgcccgcactgcgcccggccgccggctcccgcggtcccaccgtgagctcgccggcccgtcgcccgctcgccatgcaaccgccgccggcctcgcgcgcgtaggcgcccgccgcaggccatgctgcccctgctcgccgcgctcctggccgccgcctgcccgctgccgcccgtccgcggcggggccgcggacgcgcccggcctcctcggggtgccctccaatgcttcagtcaacgcgtcctccgcggacgagcccatcgccccgcggctgctggcctcggcggcccccgggccccccgagcgcccgggcccggaggagacggcggcggcagcggcgccgtgcaacatcagcgtgcagcggcagatgctgagctcgctgctcgtgcgctggggccgcccgcggggcttccagtgcgacctactgctcttctccaccaacgcgcacggccgcgctttcttcgccgccgccttccaccgcgtcgggccgccgctgctcatcgagcacctggggctggcggcgggcggcgcgcagcaggacctgcgcctctgcgtgggctgcggctgggtgcgcggtcgccgcactggccgcctccggcccgccgccgcccccagcgccgccgccgccaccgccggggcgcccaccgcgctgccagcctaccccgcggccgagccgcccgggccgctgtggctgcagggcgagccgctgcatttctgctgcctagacttcagcctggaggagctgcagggcgagccgggctggcggctgaaccgtaagcccattgagtccacgctggtggcctgcttcatgaccctggtcatcgtggtgtggagcgtggccgccctcatctggccggtgcccatcatcgccggcttcctgcccaacggcatggaacagcgccggaccaccgccagcaccaccgcagccacccccgccgcagtgcccgcagggaccaccgcagccgccgccgccgccgccgctgccgccgccgccgcggccgtcacttcgggggtggcgaccaagtgacccgctccgctcctccctgtgtccgtcctgtgtccgcgcgcgcgggtgcctttcccgccggggactcggccggtgtgcttcgtgctgtagttatcgttagttcctcttcccgagatggggccgccgagagaccccagcgcctttgaaaagcaaggtttgtgctgcgcttccagttccgaaaagcagatgtttaagcccttggactgagggtgggatcgcagctccgaagacggagaggagggaaatggggccctttcccctctattgcatccccctgcccgactccttccccgcacccacgtgccctagattcatggcagaaaatgaccaaatcctgtgtatttgttttatatatttaataactgttttaaatgaaagttttagtaaaaaaaatacaaaacaaaaagattaaattgctattgctgtagtaagagaagctctttgtatctgaacatagttgtatttgaaatttgtggttttttaatttatttaaaattggggggagggcatgggaaggatttaacaccgatatattgttaccgctgaaaatgaactttatgaaccttttccaagttgatctatccagtgacgtggcctggtgggcgtttcttcttgtacttatgtggttttttggcttttaatacagacattttcctcca

SEQ ID NO: 21 - DNA, 3970 bp, Homo sapiens
  gene (1)...(3970): ADD1; adducin 1 (alpha)
gcacccaggtcgggcggtgggggcgagcggaggggctgaggggcggagaggcctggcgggccgctgctgcgggccaggggacgggggcggagccggagccggagccgacgggcggtggccgcactgggaccccggaatcccgcgcgctgcccacgattcgcttctgaggaacctagaaagattgtacaatgaatggtgattctcgtgctgcggtggtgacctcaccacccccgaccacagcccctcacaaggagaggtacttcgaccgagtagatgagaacaacccagagtacttgagggagaggaacatggcaccagaccttcgccaggacttcaacatgatggagcaaaagaagagggtgtccatgattctgcaaagccctgctttctgtgaagaattggaatcaatgatacaggagcaatttaagaaggggaagaaccccacaggcctattggcattacagcagattgcagattttatgaccacgaatgtaccaaatgtctacccagcagctccgcaaggagggatggctgccttaaacatgagtcttggtatggtgactcctgtgaacgatcttagaggatctgattctattgcgtatgacaaaggagagaagttattacggtgtaaattggcagcgttttatagactagcagatctctttgggtggtctcagcttatctacaatcatatcacaaccagagtgaactccgagcaggaacacttcctcattgtcccttttgggcttctttacagtgaagtgactgcatccagtttggttaagatcaatctacaaggagatatagtagatcgtggaagcactaatctgggagtgaatcaggccggcttcaccttacactctgcaatttatgctgcacgcccggacgtgaagtgcgtcgtgcacattcacaccccagcaggggctgcggtctctgcaatgaaatgtggcctcttgccaatctccccggaggcgctttcccttggagaagtggcttatcatgactaccatggcattctggttgatgaagaggaaaaagttttgattcagaaaaatctggggcctaaaagcaaggttcttattctccggaaccatgggctcgtgtcagttggagagagcgttgaggaggccttctattacatccataaccttgtggttgcctgtgagatccaggttcgaactctggccagtgcaggaggaccagacaacttagtcctgctgaatcctgagaagtacaaagccaagtcccgttccccagggtctccggtaggggaaggcactggatcgcctcccaagtggcagattggtgagcaggaatttgaagccctcatgcggatgctcgataatctgggctacagaactggctacccttatcgataccctgctctgagagagaagtctaaaaaatacagcgatgtggaggttcctgctagtgtcacaggttactcctttgctagtgacggtgattcgggcacttgctccccactcagacacagttttcagaagcagcagcgggagaagacaagatggctgaactctggccggggcgacgaagcttccgaggaagggcagaatggaagcagtcccaagtcgaagactaagtggactaaagaggatggacatagaacttccacctctgctgtccctaacctgtttgttccattgaacactaacccaaaagaggtccaggagatgaggaacaagatccgagagcagaatttacaggacattaagacggctggccctcagtcccaggttttgtgtggtgtagtgatggacaggagcctcgtccagggagagctggtgacggcctccaaggccatcattgaaaaggagtaccagccccacgtcattgtgagcaccacgggccccaaccccttcaccacactcacagaccgtgagctggaggagtaccgcagggaggtggagaggaagcagaagggctctgaagagaatctggacgaggctagagaacagaaagaaaagagtcctccagaccagcctgcggtcccccacccgcctcccagcactcccatcaagctggaggaagaccttgtgccggagccgactactggagatgacagtgatgctgccacctttaagccaactctccccgatctgtcccctgatgaaccttcagaagcactcggcttcccaatgttagagaaggaggaggaagcccatagacccccaagccccactgaggcccctactgaggccagccccgagccagccccagacccagccccggtggctgaagaggctgccccctcagctgtcgaggagggggccgccgcggaccctggcagcgatgggtctccaggcaagtccccgtccaaaaagaagaagaagttccgtaccccgtcctttctgaagaagagcaagaagaagagtgactcctgaaagccctgcgctaacactgtcctgtccggagcgaccctggctctgccagcgtccccggccacgtctgtgctctgtccttgtgtaatggaatgcaaaaaagccaagccctccgcctagaggtcccctcacgtgaccagccccgtgtagccccgggctgacccagtgtgtgctcagcagccccaccccaccctgccccttgtcctctcagagcctcagcttctgggggagacatgctctccccacaggggggaggcactaagtcatggtcctggctggaaggtactgaaggcttctgcagctttggctgcacgtcaccctcctgagcctcacctttcctgccgtccctcctgttgtgaaatcaccacattctgtctctgcttggcttcccctccaccctaaagtctcaggtgacggactcagactcctggcttcatgtggcattctctctgctcagtgatctcacttaaatctatatacaaagccttggtcccgtgaaaacactcgtgtgcccaccagcggccttgaagaggcaggtctgggccagatgctgggcaggaaaccccagcggcagatgggcctgtgtgcacccaacgtgatgctatgcatgtctgaccgacgatccctcgaccagaatcagattcaggagctcagtttctttttcacttgggtctctggattcctgtcatagggaaggtatatcaggaggggaagaggcctttctagaattttctttgagcaggtttacaatttagcttacatttttcgactgtgaacgtgaataggctgctttttgctttcttctttccagaccccacagtagagcacttttcacttatttgggggaggcttcaggggactgttctcaccttaactcagccagaaagatgccctagttgtgatcaaaggtaactcgaggtggagggtagccctggggcccctcgacatcaccgtcattgatggagcctgaaccgtgtgctcctcggcagatgctgttgttgttacttccctccaagaggctggaaaagggctcagagctgctgagcaggaaccggagggtgacccatttcaggaggtgccggtaccagcctgactaggtacaggcaagcttgtgtgggcccaacaggcccttggtagagctggtgccagatgtgggctcagatcctgggcatgatgggccgagccacctcggatcccactgattggccagccgagcgagaaccaggctgctgcatggcactgaccgccgcttccagcttcctctgagccgcagggcctgctacgcgggcaagcgtgctgcctctcttctgtgtcgttttgttgccaaggcagaatgaaaagtccttaaccgtggactcttcctttatcccctcctttaccccacatatgcaatgacttttaattttcacttttgtagtttaatcctttgtattacaacatgaaatatagttgcatatatggacaccgacttgggaggacaggtcctgaatgtcctttctccagtgtaacatgttttactcacaaataaaattctttcagcaaaaaaaaaaaaaaaaaaaaaaaaaaaaaaa

SEQ ID NO: 22 - DNA, 1472 bp, Homo sapiens
  gene (1)...(1472): GPSM3; G-protein signalling modulator 3 (AGS3-like)
ccggagcggggatcaccttaacttgactgtggtcccccagccctccaagaaatgcccataaaccaaggaggagagggtaaaaaatagaagaatacatggaattggggagaagaatgggaaaaaagatgggagtgactgcataatgtcagcattttgtgcttttggctcagcatttggattggatggaggattttcatcctgaaaccagccctccgtcgccaccgaccatggaataattgtcttccacgaaactcttccctggtgccaaaaagggttttatgtatatatttgagagctccacatccctggatctgaatcctccacttcccactggaaccatgcccctcccagtcccgtttctcccctctaggggatggaggctgagagaccccaggaagaagaggatggtgagcagggcccccctcaggatgaggaaggctggccccctccaaactccaccactcggccttggcgatctgctcctccatcccctcctcctccagggacccgccacacagccctgggaccccgctcggcctccctgctctccctgcagactgaactccttctggacctggtggctgaagcccagtcccgccgcctggaggagcagagggccaccttctacaccccccaaaacccctcaagcctagcccctgccccactccgtcctctcgaggacagagaacagctttacagcactatcctcagtcaccagtgccagcggatggaagcccagcggtcagagcctcccctccctccaggggggcaagagctcctggagttgctgctgagagttcagggtgggggtcgaatggaggagcaaaggtcccggccccccacacacacctgctgagacttgagccccaaccagcccttccttgccactggtctcaaagctgggcagcccattgcatgccctcaactcttgcttggcaggggtaccagagactgaaagacacggcacaaatctcaatattcatctcccacatcaccttccctgggaactggacagggtgaaagtcctcaaactctgggaacaggcgagatggaacagggatttaactccccgcccacaggtccatgggagcttgaggcagtaagggggatcccaggcacccatctcaaggagtggctgggagtcttttccctaacttgtggggacaccaccagttgtcaagctactaggcagtagggtctgagggctcaggcctccacctgagaggttataacctgagagacagctctacccttcctcccagtaagaagggaaggtgggtgggcacctgagagattaagactattctcccagtcccactaccagcacccccgatccctgagactgaggggtttacgggctgtgaatggaccttcagccctgcccaccctccctccccactgctgctgagtctgtctgatgttttggttgtgtgaataaatataattcccctctggaaaaaaaaaaaaaaaaa

SEQ ID NO: 23 - DNA, 2680 bp, Homo sapiens
  gene (1)...(2680): BC002942; hypothetical protein BC002942
gtagcggacatggcgggctcccggctcccgcggcagctcttcctccagggcgtggcggccgtcttcatgtttgctttcgcttccctctacacgcaaatcccaggctgggggatctgcaggccctgactcagaggtagtctagtctgcaagggctgtccagggcctggccatgacatgccctcccacaggcctgtatggccccgagggcatcctacctgcaaggaggacgctgcggcctcagggcaaggggcgctggcagcagctgtgggagaccccgacgctgctgtgggaagcgccgagactggggctggacacggcccagggcctggagctgctgagcctgctgggtgcactagtggccctgggagccctgctgctgagcccactgcgccaccctgtcatctacttgctgctttgggccgcctacctgtcagcctgccaggtgggccaggtgttcctttatttccagtgggactccctgctgctagagactggcttcctggccgtgctggtggccccgctgaggccagcctcccaccgcaaggaggccccccagggcaggcaggcaggggccctgccccacgaagacctccccttctggctggtgcgatggctgctgttccgcctcatgttcgcctcaggcgtggtcaagctgaccagccgctgccctgcgtggtgggggctcactgccctcacctaccactacgagacccagtgcctgcccacgcccgccgcctggttcgcacaccacctgccggtctggctgcacaagctcagcgtggtggccaccttcctaattgagatcgctgtgccgcccctgttcttcgcccccattcgacgcctgcgcttggctgctttctactcgcaggtgctgctgcaggtcctgattatcatcaccggcaactacaacttcttcaacctgatgacgctggtgcttaccactgcgctgttggacgaccagcacctggctgctgagcctggccatggcagccgcaagaagacggccacctcctggcccaaggccctgctggccaccctgtcgctgctgctggaactagccgtctacgggcttctggcctatggcactgtgcactactttggcctggaggttgactggcagcagcgcaccatccactccagaaccactttcaccttccaccagttttctcagtggctgaagacactgacgctgcccactgtgtggctgggtgtggcctccctggtctgggagctgctgagtgccctgtggaggtggacccaggtgcggggctggctacggaagctcagtgctgtagtccaactgtcccttgtgggcactgcgaccgtggccttgttcctgattagcctggtgccgtactcctacgtggagcccgggacccacgggcgcctctggaccggggcccaccgcctgtttggtgccgtggagcacctacagctggccaactcctacggcctcttccgccgcatgactgggcttggtggacggcctgaggtggtgctggagggcagttacgacggccaccactggacggagatcgagttcatgtacaagcctgggaacctgagccggccgcccccggttgtggtgccccaccagccacgcctggactggcagatgtggtttgcagccctgggcccacacacgcacagcccgtggttcacaagcctggtcttgcgcctgctgcagggcaaggagccagtgatccgccttgtccagagccaagtggccaggtatcccttccacaagcagccgcccacctacgtccgagcccagcgctacaagtactggttctcccagcctggggagcagggccagtggtggcggcgccagtgggtggaggagttcttcccatccgtgtccctgggggaccccacgctggagacgctgctcaggcagtttggactacaggagaaaagcccacctcgcacccgcagcgccaacagcaccctggcccaggccctccactggactcgctctcagctgtctcccctggaggcccccgccctgctctgggggctcctcatggccgtgggggctgtcagatttgtgcaggccctgctagcaccctgttctctccggtcctccccgctggcaccagtcagcggggagaagcgcaggccagcctcccagaaagactccggagctgcctccgaacaggccaccgcagcccccaacccctgctccagtagttcgaggaccacccggcgaaagaagtagctgtgttctcccagctgcacgtcctgagagggccaggtcgccgggagtgctctggcctccggcaggacaggacccagccactgtgccttagctgaccctgcagggccaggcacaggttggggggctgcccctggggtttgcagggtgctgcattgagggctccaggccccacccccacgccagccatgcccctccccaggactcccactattgcctctgtgattggcccaggaggaaaacacgaccaagctcaagacccttcccctgccctgggctgtgggggtctgagtctagagcccccaaccctaggccccgtgccagaggggaagaggctgactcccaggggaagaggggaagcactgtcatcttccacgtcatcttcacaccagcccatcctgccctttagatctgggcaccaataaaggcgtcttttgtgcttgaaaaaaaaaaaaaaaaaaaaaa

SEQ ID NO: 24 - DNA, 1177 bp, Homo sapiens
  gene (1)...(1177): TNFRSF5; tumor necrosis factor receptor superfamily, member 5
gccaaggctggggcaggggagtcagcagaggcctcgctcgggcgcccagtggtcctgccgcctggtctcacctcgctatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacgtgggcaaacaggcagttggccagagagcctggtgctgctgctgctgtggcgtgagggtgaggggctggcactgactgggcatagctccccgcttctgcctgcacccctgcagtttgagacaggagacctggcactggatgcagaaacagttcaccttgaagaacctctcacttcaccctggagcccatccagtctcccaacttgtattaaagacagaggcagaaaaaaaaaaaaaaa

SEQ ID NO: 25 - DNA, 7121 bp, Homo sapiens
  gene (1)...(7121): N4BP1; Nedd4 binding protein 1
ggcccggcccgcggcggcggcaaaggggcggaggggttcgctgccagcaattgccaagctgctgcggtcgtggcgggatggtatagccgggcctgagggcgggcggcggggcgcgggcgggggcgaggcggccgggaagggccgacgccgcctgactggcttgggagcagcggcagcaagctgagggggcgtcgccgccgccggcccccggcgccgccgcgggaggccgcgcccgccatggcggcccgggcggtgctggacgagttcactgcgccagctgagaaggcggagctgctggagcagagccgcggccgtatcgagggcctgtttggcgtgagcctagccgtgctcggcgcgctaggggctgaggagccgctgcccgcgcgcatctggctgcagctctgcggggcgcaggaggcggtgcacagcgccaaggaatatattaaaggaatctgtgaacctgaactagaagaaagagaatgttaccccaaggacatgcactgcatttttgttggggcagagagcctgtttctgaaaagcttgattcaggatacttgtgctgacctctgcattctggacattggccttcttggcatcagaggaagtgctgaggctgtggtcatggctaggagtcacattcaacaatttgtaaagctctttgaaaataaagagaacctacccagtagtcagaaagaatcagaggtgaaaagggaattcaaacaatttgttgaagcccatgcagacaattacacaatggatttgttgattttgcccacttccttgaaaaaagaacttttgacactcacacaaggtgaggagaatctctttgaaacaggagatgatgaggttattgaaatgagagattctcaacaaacagagtttacacagaatgctgccacagggctgaatatttctagagatgaaactgttttgcaggaagaggcaagaaataaagctgggactcctgtttctgagcttacaaaacaaatggacacagtcctttctagctcaccagatgtgctttttgatccaataaatggtctaaccccagatgaagaggcactttccaatgagagaatttgtcagaaaaggagattttctgattctgaagaaaggcatacgaagaagcagttttctttggaaaatgttcaggagggggagattttacacgatgctaagacattggctggaaatgtaatagctgacctatctgattcttctgctgattctgaaaatttaagtccagatataaaagaaactactgaggaaatggaatacaacatcctcgtaaacttttttaaaaccatgggctactcccaagaaattgttgaaaaggtcattaaggtgtatggaccatctactgaaccattattgctcttagaggaaattgaaaaagaaaataaaagattccaagaagacagagaattttcagctggtacagtgtatccagagaccaacaaaaccaaaaataaaggtgtttatagcagcacaaatgagcttacaactgattccactccaaagaaaacacaggctcacacacagcaaaatatggtagaaaaattttctcagttaccattcaaagtggaagctaaaccatgtacctcaaattgtagaattaatactttcagaacagtgccaatagaacagaaacatgaagtctggggttcaaaccagaactacatttgtaacacagaccctgaaactgatggcctttcaccttctgttgcctctccaagtcccaaagaagtcaattttgtttcaaggggagcttcaagtcaccagcccagagttccactttttcctgaaaatggtttacaccagcagccagaacccttgcttccaaataatatgaaatctgcctgtgaaaaacgtttaggatgttgtagttctcctcattctaagccaaattgctcaaccctttctccaccaatgccactgccccagctgttaccttcggttactgatgcaaggtcggcaggaccttctgatcatattgattcctcagttactggggttcaaaggtttcgagatactctaaaaataccctacaagctggaattaaaaaatgaaccagggagaacggatttgaaacacattgttatagatgggagcaatgttgcaattacccatggtctgaaaaagttcttttcttgtcgtggaattgcaattgcagttgaatatttttggaagcttggcaacagaaacatcactgtatttgtccctcagtggagaacaaggcgtgatcctaatgtcacagaacagcacttcttaacccagctccaggagctcggaatattatctttaactcctgcccggatggtctttggagaaagaattgcttctcatgatgacaggtttctactacacttagcggacaaaactggtggcataattgtgacaaatgataatttcagagaatttgtgaatgagtcagtctcttggagagaaattattacaaaaaggctgctgcagtacacgttcgtgggggacatatttatggttcccgatgatcctctgggaagaagtggacctcgattagaggagtttcttcagaaggaagtctgtcttagagatatgcagcccctactcagtgccctgccaaatgtgggcatgtttgaccccagcttcagagtccctggcacccaggcagccagcaccagccaccagcctccgacccggattcagggagccccttcaagccactggctccctcagcagccccacttcccactgctgccagccctccccagtctccagcagaacctgcccatgccagctcagagatcttctgcagaaaccaacgagctgagggaagcccttctgaagatcttccctgactcagagcaaagactgaaaatagaccagatcttggtggcccacccatacatgaaagatctaaatgcgctttctgccatggtgttggattgaagattgtgctgagcgctgagcttgcgctcagggctggctgatcaaactcaagctcacacctgtacctgatgggaatgaacataatgtgaagaaacttatttcccagtgtaaacgcagttgtacagaaaaaaaatgttttgggtataaatctgggttttcaaaaccagcttttttctatatataaattatgctgctattacagatttaacattttctgttaaaggaaagtctacattttctgcctgttcagaactgtttaatagcagttactcttgagtgtatttacctttttatgttttttaaactattttccttaaagccgaaaatattgacaaatggatatggttagcctttataaataaataaaaaaaaaagtgttgctttcttaaggaaagcaagacgtcttaaagtatattttcttgagatgagtatgtcccatcactaccatgaagtgtgctcacccttccagcctcctctgccctcacccccggagttaaagtgggtaagagttggtttgttctgcagtctttggttggagtgttgtgaaagtggacccgcggtgccactagatggcacttggtgcctagccatggtgaatgaccagggcgaagttagtctcacagaataccagggctagaagacatcgtggagcccatggagtcccaccctccttcccccttccaccctgcgcaaaaggggacggcccagtgtgcacgccgtccggccatgcccacagccacagccacagctgaggagtggccgggcaggcagcaggctccccagccagggcttgacacgcccacagtcaggggcctccacatgcctggcctggctctggaagtcacgtcttagcttctgaacaccgtagcgggttatgaggaagtttaatgaagagcccggtcactgtggccatcttgtgtccccaactgcaggagacgccctgatgtggagtttgtacagctttgccaaaaaatggttttctattctcaaaagtgacccaagccaataaatagcattagtagcttctgtggggggatcccagagcccctgtatttattttccctgtgtttgtctctagtgtcctcctaaacagcctttcctgtgagtctttctcagaattgatatcttaatattgtctgttctaggtgttgcccaaattcagtgtcagtgaagttcttttcctgggcaatcttaacatctttactcttattgtctggaccttcaaagggctttgtattttacacctgcgcccccaggctgctcacagtcggcttgctgcctgtcctgcctgtccggatcttgtctcctagtcctgaggagcggaggctgagaactcagtgctggtttgtaaatggtgaggggacttgggggcatcccagagtgctccctccaggcctgcttcttggttttgtttgatcactgcgttcttcaagggatgaatccagagccctccatgaggccaagcttgtccttcaatcatgtttcctctcagatgcgtccgtgatgcctcctaatgtggaactggttgtccattgtttgggcctatggccaagtcacccagctgtggaagcagaggtagaagacgaggccagccaggagggcgacttcagtcacagctcccatgcctcagctttgtacctgttttcaaaagcacaactgaggtgtgcgggctggagctgtcttgcagtgattctggccttctggctcatggttcagtccagcagcctggctgacccactatttctcctctgcttcagaggaaacccaggaaatgcccttactgccaggctgagtctccacccatgctggttggtgctggctaggctgagggggccaccacttttcctggctagaagctacttgacctttgatgtttgagttctgtaagtcttcgtgttctgacttactgcttcagagggattggcctgtcccctttccctttctcggctatgggaaggaaggattgctcattggttgccttcatcagttacagcatgagacggaattcatcattccttccgaaacccctgatatttaatatttaatatttaaaaacccaaattatcaaaccattaagaactcattactggttctcagcctcctccagtactagcctcagtgtggctgctgcataagtatctgtagcctgtctacctcctgcagtggggccgctcgcctcttccctgtctactgctcaggctctcccacttcgtggcatccatgtaaagtaggtggcagggcagagatgtcactctcattcaacagggaggatgtctgttgctcagagaggttgtcctgaggggctgggtgattcctgggcctacattcttcccgaggctccaggccgctgtctctggaagtaaaagagccttgtctgaccttaatgcaagcagtctgtttgaacccctgtaggctgcattcaggagacagaaggtgtctgggccatcctgggcggccggtcagcgttgctaggcaggctcggctgtctggccgggacttgggcctgggtggcttttgagaccagtgaagaagggagagccggcctcatgccgatgcggcttgtggcacggctgggatgtgagggaggactcagatctacacacagaaacccctcttctccccgccctcccccagctcctacctgcctcccacgcctcaggtgtggctgcctgtgggaccatcccccaacccctttcctcgcacctccttgtcctcacccagttcctgcagtgtctctgacccacgcctcccgcctcctggccgacttgcccaggaggtgtctctggctcacctccgtctgttcatcaccttcctccccagtgtttccacttatcttggatgttttagattgaaacagcctgattcccggaagaatcctcttcattcattgctagtgcttcccctcacctcccactctccacttcccagtttgcaaatgtggctttcgcccaccaagtgaaagcggactgagagcagcccttggggacggcccggtgcctggctgcaaggccgcgctggggctctgtcttggtgcacatggcttgaccggactttccctgcttcccaccacttccctcactcccagacctccctcattctttttgtctcttctttttgcctaaagccagtccttaacaccctattcttcctctgcaggtggcttgcagacttttccccacctttggggctcgtggtggtggagagggcagctggtgttaagaatgtaggttaccgggcatgacccggcagatgcttgcccagtagttctggaggaaggcccgggaatctgcaaatgagcgcattccccaggcagttcccatgcaggtgatccacggaccacatgttgagaaactgcagtcacccttagggccacaccgtccctctcctcgctgtcccctctctgtagtgactggccctgaccttcaggagtgcactttccactctaccaggaagccctatgacatcctcaggctccccagacctgcagcttgcatggggcccctcccttcttccacacccaccctccgtatggtcccctgctctgccctcgtgctttgctggcccctgcccgctactcccactctcagacacccaggggtggtgggccctaactggctggcccctcccagcgctgccctctgccgtccagatgctgcagtgtggccagatttaccttccagtaacatacttctagtcacccctcctcctgcgaagtgatctgcagtggctgtttgaccagaccacaaagttcacatctcctgagcttagtgtccgtggctgtccacctcccagccatacttgactgtccccaaactctccctgcagccacatgtttcccatgacctgtgggctctgcagatggacctctctccgctagagatgcccttctcccaaatggcttccctcctggaaggcccagcctgagtcctcgtctcctttccagtgcttctgccagaagcatccccatgatgttgtgaccgcacagcactttgtgtctcgctttgagcacttgccactctggctggtgctgctgccactgattgtgtactgtcttgctgccctttctagactgtgagctcctcgtgggcagggaccgcctgtgttctctgtatttcccacggcgcctagcacagtgccttgcacttgataggtgcttaataaatgtctgctcaactgaaaaaaaaaaaaaaaaa

SEQ ID NO: 26 - DNA, 1575 bp, Homo sapiens
  gene (1)...(1575): FLJ12438; hypothetical protein FLJ12438
aaaagaccaatcaagacggccggccgaggcccctggaacggcttaggcggctgcggctgctacggcggcgcatgctaggggattctgccgggtagaagagctgggcctggaacccagccctgaggacatcctgcggcccaggggcaagtgacacctgctgagagaggcccaggatggtggaggctgaggaactggcacagctgcggctgctcaatctggagctcctgaggcagctgtgggtggggcaggatgctgtgcggcggtcagtggccagggcagcctcggagtcaagcctggaatccagcagcagctacaactcagagactccatcgaccccagagacgtcctcaacttccttgagcacctcctgcccacggggccggtcctccgtgtggggcccaccagatgcctgtcgaggggacctccgtgatgtggccagatcgggggtggcctctctcccacctgccaattgccagcaccaggagtccctgggccgaccgagaccccactcagcaccctcgctgggcacctcaagcctgagggacccagagccctcagggaggctgggtgatccaggaccccaggaggcacagaccccgaggtccatcctggctcaacagagcaagctgtccaagcccagggtgaccttctctgaggagtctgcagttcctgagaggagctggcgcctcaggccatacctgggctatgactggattgcagtgtctctggacaccagctcttccatcaccagccagcctgaggccttcttctccaagctgcaggagtttcgggaaaccaacaaggaggagtgtatctgcagccatcctgagccccagttgccaggcctgcgtgagagcagtggcagcggcgtggaggaagaccatgaatgcgtgtactgttaccgtgtcaaccggcgcctgttcccggtgcctgtggatcccggtaccccctgccgcctgtgcaggacaccgcgagaccagcagggccctgggaccctggcgcagccagcgcacgtcagggtgagcatcccgctgtcgatcctggagcccccgcaccggtaccacatccaccggcgaaagagctttgacgcctctgacacactggccctgccccggcactgcctgctgggctgggacatttttcctccgaagtctgagaaaagctcagcccccaggaacctggacctctggtcctctgtctccgctgaggcccagcaccagaagctgtccggcaccagcagcccttttcacccggcctcaccaatgcagatgctgcccccgaccccgacctggtcagtgccccaggtccctcggccccacgtcccacggcagaagccctgaggactgactcctgggggagaacagcattcccgccgcctccagcctctcccctctggcaggcgcacccaggagatggaatcccctgcccgcccagctcaggcccagctgtcctaggttgggcaggtgggtggacccaagcttgtctgctgcctgagttccagagagggaggaccctggggtggagggtgagggattctgtggaagtttgtaaataaagctcagtgctctgcagctc

SEQ ID NO: 27 - DNA, 2623 bp, Homo sapiens
  gene (1)...(2623): MMP24; matrix metalloproteinase 24 (membrane-inserted)
gggatgccgaggagccggggcggccgcgccgcgccggggccgccgccgccgccgccgccgccgggccaggccccgcgctggagccgctggcgggtccctgggcggctgctgctgctgctgctgcccgcgctctgctgcctcccgggcgccgcgcgggcggcggcggcggcggcgggggcagggaaccgggcagcggtggcggtggcggtggcgcgggcggacgaggcggaggcgcccttcgccgggcagaactggttaaagtcctatggctatctgcttccctatgactcacgggcatctgcgctgcactcagcgaaggccttgcagtcggcagtctccactatgcagcagttttacgggatcccggtcaccggtgtgttggatcagacaacgatcgagtggatgaagaaaccccgatgtggtgtccctgatcacccccacttaagccgtaggcggagaaacaagcgctatgccctgactggacagaagtggaggcaaaaacacatcacctacagcattcacaactataccccaaaagtgggtgagctagacacgcggaaagctattcgccaggctttcgatgtgtggcagaaggtgaccccactgacctttgaagaggtgccataccatgagatcaaaagtgaccggaaggaggcagacatcatgatcttttttgcttctggtttccatggcgacagctccccatttgatggagaagggggattcctggcccatgcctacttccctggcccagggattggaggagacacccactttgactccgatgagccatggacgctaggaaacgccaaccatgacgggaacgacctcttcctggtggctgtgcatgagctgggccacgcgctgggactggagcactccagcgaccccagcgccatcatggcgcccttctaccagtacatggagacgcacaacttcaagctgccccaggacgatctccagggcatccagaagatctatggacccccagccgagcctctggagcccacaaggccactccctacactccccgtccgcaggatccactcaccatcggagaggaaacacgagcgccagcccaggccccctcggccgcccctcggggaccggccatccacaccaggcaccaaacccaacatctgtgacggcaacttcaacacagtggccctcttccggggcgagatgtttgtctttaaggatcgctggttctggcgtctgcgcaataaccgagtgcaggagggctaccccatgcagatcgagcagttctggaagggcctgcctgcccgcatcgacgcagcctatgaaagggccgatgggagatttgtcttcttcaaaggtgacaagtattgggtgtttaaggaggtgacggtggagcctgggtacccccacagcctgggggagctgggcagctgtttgccccgtgaaggcattgacacagctctgcgctgggaacctgtgggcaagacctactttttcaaaggcgagcggtactggcgctacagcgaggagcggcgggccacggaccctggctaccctaagcccatcaccgtgtggaagggcatcccacaggctccccaaggagccttcatcagcaaggaaggatattacacctatttctacaagggccgggactactggaagtttgacaaccagaaactgagcgtggagccaggctacccgcgcaacatcctgcgtgactggatgggctgcaaccagaaggaggtggagcggcggaaggagcggcggctgccccaggacgacgtggacatcatggtgaccatcaacgatgtgccgggctccgtgaacgccgtggccgtggtcatcccctgcatcctgtccctctgcatcctggtgctggtctacaccatcttccagttcaagaacaagacaggccctcagcctgtcacctactataagcggccagtccaggaatgggtgtgagcagcccagagccctctctatccacttggtctggccagccaggcccttcctcaccagggtctgaggggcagctctggccagtgctcaccagggccagcagggccctaggctggggtcgtacagctgaagttgtgggtgcattggcctaggctgagcgtggggcagggaattatgggggctgtgcccagggtgggtgtctggcacccagctgccagccttctgtcctgggcaaactactccctacttaagggaataggccaggctccatccggaggcagggaccatgccaggaggagcccctgtggtcacggcatcctgtggtgtccatgaggtaccacagctccactcctggctggaacccggcaccctctgtgggaagccagcactagctctcatcccccatccgggagataccaccagtcctggtccccttttgccaacacctgctggtcagatgtccccctacccccaccccactgtcctccaaggctacaggacccctgcttctgacacagtgagcaacaagcctgggtttccctgctggcagacggcagatccctcaggaaacctgctccacttgtcagggtctcttcggagacccaggatttagggtcacatgctgcaggcagggctgtggcccagctgggtctgacaaggacccgtgtcacatcgtgaatattta

SEQ ID NO: 28 - DNA, 4394 bp, Homo sapiens
  gene (1)...(4394): PTP4A1; protein tyrosine phosphatase type IVA, member 1
ccggctcggtacgcgctctgctccgagccgctcactgcgtggtagagtctggtgcccccgccgtcgcctgcatcgccgccaccgccgctccgccacgaccaccgccgcctcctgccctgcagccaccgccaccgcctgtgtcgccgccgcctcgggaccggctgtatgattaggccacaatcttcaatgagtaaacatattcctcaattctgtggtgttcttggtcacacatttatggagtttctgaagggcagtggagattactgccaggcacagcacgacctctatgcagacaagtgaactgtagaaactgattactgctccaccaagaagcccccataagagtggttatcctggacacagaagtgttgaattgaaatccacagagcattttacaagagttctgacctggatggggtaaacctcagtgcacttcttttctgttggcctcagtattactggattgaagaattgctgcttcttgttaggaggttcatttcacttatcattacttacaacttcatactcaaagcactgagaatttcaagtggagtatattgaagtagacttcagtttctttgcatcatttctgtattcaatttttttaattatttcataaccctattgagtgttttttaactaaattaacatggctcgaatgaaccgcccagctcctgtggaagtcacatacaagaacatgagatttcttattacacacaatccaaccaatgcgaccttaaacaaatttatagaggaacttaagaagtatggagttaccacaatagtaagagtatgtgaagcaacttatgacactactcttgtggagaaagaaggtatccatgttcttgattggccttttgatgatggtgcaccaccatccaaccagattgttgatgactggttaagtcttgtgaaaattaagtttcgtgaagaacctggttgttgtattgctgttcattgcgttgcaggccttgggagagctccagtacttgttgccctagcattaattgaaggtggaatgaaatacgaagatgcagtacaattcataagacaaaagcggcgtggagcttttaacagcaagcaacttctgtatttggagaagtatcgtcctaaaatgcggctgcgtttcaaagattccaacggtcatagaaacaactgttgcattcaataaaattggggtgcctaatgctactggaagtggaacttgagatagggcctaatttgttatacatattagccaacatgttggcttagtaagtctaatgaagcttccataggagtattgaaaggcagttttaccaggcctcaagctagacagatttggcaacctctgtatttgggttacagtcaacctatttggatacttggcaaaagattcttgctgtcagcatataaaatgtgcttgtcatttgtatcaattgacctttccccaaatcatgcagtattgagttatgacttgttaaatctattcccatgccagaatcttatcaatacataagaaatttaggaagattaggtgccaaaatacccagcacaatacttgtatatttttagtaccatacagaagtaaaatcccaggaactatgaacactagaccttatgtggtttattccttcaatcatttcaaacattgaaagtagggcctacatggttatttgcctgctcactttatgtttacatctcccacattcataccaatatacgtcaggtttgcttaaccattgatttttttttttttttaccaagtcttacagtgattattttacgtgtttccatgtatctcactttgtgctgtattaaaaaaacctccattttgaaaatctacgttgtacagaagcacatgtctttaatgtcttcagacaaaaaagccttacattaatttaatgtttgcactctgaggtgcaacttaacagggagggcctgagaaaagaatgggagggggctattaattattttttagcaaaatgttgcctttgtcttgtgcaaacatgtagaatatgctctttaatttagtaaaatatttttttaaaaggtagagatgctttgttattgtaatcataaacttcctgaaattcttgtaatttttttcccatacttatcagaagtgtgtttaccaacttatttttgtttgaaagtgtgattttttttttccttcccaacctctcttgcaaaaaaagaaatgggtttctgctaatgaattgagcacatctaatattttatatgccttttggagctgtgtaagttaatatttgatacttgacaatttgttttattatgtaattgataaaatggtgatgtgtattaatgttagttcaaccatatatttatactgtctggggatgtgtggttatagttctgtgggagaaataattttgtcagtgttcaccagcttgtaaaaacttagtgcgagagctgaaacatctaaataaataatgacatgcatttatcatcattgagattggtttgctttaaattaacttattttgtagaagacaaaatgaattgcacttcacttaatgtgtgtcctcatctttttacaaataaatgaaggattataaatgatgtcagcattttagtaaacttttagacaaaatttgttagggtcattcatgaaaactttaatactaaaagcactttccattatatactttttaaaggtctagataattttgaaccaatttattattgtgtactgaggagaaataatgtatagtagaggacagccttggtttgtaaagctcagttccactagttcatggttttgtgcaacttctgagcctcagttttctcctttgcaaattaataattacatacctttatagattttgaaattaatttaaatattagtatttggtacatgaaggcttaatgttaagtttcctttaatgatccacaataatccctttgatcacgttaatctaaatctagatgtctttgtctaattttttttgaatagcagttataaatgtaaaggactcaaagtttaagtaaaaagtgatactccaccttgtgtttcaaagaatttagttccacctcttcataccagtttaacacttaatatatttcattggattttagacagggcaaaaggaagaacaggggcctctggaggcccttggttatttaaatcttggattatttgtgatagtaatcacaaatttttggctaatttttaacctgaggttttgttttttttttaaaggaaatgcagcctagtcttgagaacataattttatataatcaattactaaatgttaaactattaccacacagcccataaaacagcatttgcgtttattgagagagaggatgtgccatcatgattaatgaaaactatcttttgagtttgaaaagaaattaatttgcagtgtttggattgtatatatggtgctaaaaataaattaatttactttataaaccttatctgtacattatacgatgtgatgaaatttgctttttatccaaatattttgtatcttgtaaatatggctaattataggaatgcctataatacatcttagattccttatatctaataagagttcaaagagttatgagttgaagtcttgaatgcaggaaactatctgatagtgttctaaaatttggttacttgggtttggatacccttagtgggatgatgtaaatagaggctagctacctaggcttgtctatagcaaccataatgttgatgtaagtaatgcggttactgaatcataagaaaatgccatctctttttagttgaaggaaaactctggaagtaggtgccattggtcattctgcagtgcactgcaaccattgtttcccctagtgccctcttttccctagggcattgctctcctattcccacgccttaacacagctctatacctagaagcagccagcccaggcatgcagtcacatttaatcacatcccccttctagagtgcttcaaaatgatgtagtccctcaacttggctaaagaatctcaatctcttgaaatttatttttttaatgtcatattcatctggtaaatatctactgtttgccaggcatttaagaatatggcaaagaacataaaagatggtgtcaccagattttggtcaccaatgagtacccgacccgttgccatgattaagagagaatgctttctattggagtttcaggaaatataatttgagaatactttaaagggaagtggaagtataagtgaatgatatttttcttttacatgtaaacaatgaagttatttcaaagttaagttttaaacaaaatacatgaagtagtgtctgccatacatgttaatattctacattcttgcttccttaaattaatatgtttgtgtgtatatatgtgcctcacacctgaattgaaaattaaagactggtttaaaagtggttaaaaaaaaaaaaaaaaaa

SEQ ID NO: 29 - DNA, 5581 bp, Homo sapiens
  gene (1)...(5581): PAFAH1B1; platelet-activating factor acetylhydrolase, isoform Ib, alpha subunit
agctcctgtgacagacggagctggagcggcggggcggcggcggagtccggcggccgggagagcgagtgagcgagcggaggagcagcgacacgggagtctagggagcgagaaggagaaggaggggagcgctcgggcgcgagcgagagaaaccgcgagcgccgagcttggactcgagccccggaacggctgaggagcccgcccgctcccctcccctccccctccccgggcccgggcccagcgcgccatcctcccccctccttccctccctccctccttcctccctcccctctccctccccctcccccgccggtggatgggagtgaaggacggaagaggcccggcggaggcggcggtgcagcgctccggtggaatgaatcttacttgttgaatatcttctggttactagttggattcatttgtgaaagaatcattttcccctgtgtggaagacacttagtggcatatttaaattataagtccacggatcaaaaagctttttgatttcccaaaggagggacataccactatatcagataagcttgacattacagccaagatggtgctgtcccagagacaacgagatgaactaaatcgagctatagcagattatcttcgttcaaatggctatgaagaggcatattcagtttttaaaaaggaagctgaattagatgtgaatgaagaattagataaaaagtatgctggtcttttggaaaaaaaatggacatctgttattagattacaaaagaaggttatggaattagaatcaaagctaaatgaagcaaaagaagaatttacgtcaggtggacctcttggtcagaaacgagacccaaaagaatggattccccgtccgccagaaaaatatgcattgagtggtcacaggagtccagtcactcgagtcattttccatcctgtgttcagtgttatggtctctgcttcagaggatgctacaattaaggtgtgggattatgagactggagattttgaacgaactcttaaaggacatacagactctgtacaggacatttcattcgaccacagcggcaagcttctggcttcctgttctgcagatatgaccattaaactatgggattttcagggctttgaatgcatcagaaccatgcacggccatgaccacaatgtttcttcagtagccatcatgcccaatggagatcatatagtgtctgcctcaagggataaaactataaaaatgtgggaagtgcaaactggctactgtgtgaagacattcacaggacacagagaatgggtacgtatggtacggccaaatcaagatggcactctgatagccagctgttccaatgaccagactgtgcgtgtatgggtcgtagcaacaaaggaatgcaaggctgagctccgagagcatgagcatgtggtagaatgcatttcctgggctccagaaagctcatattcctccatctctgaagcaacaggatctgagactaaaaaaagtggtaaacctgggccattcttgctgtctggatccagagacaagactattaagatgtgggatgtcagtactggcatgtgccttatgaccctcgtgggtcatgataactgggtacgtggagttctgttccattctggggggaagtttattttgagttgtgctgatgacaagaccctacgcgtatgggattacaagaacaagcgatgcatgaagaccctcaatgcgcatgaacactttgttacctccttggatttccacaagacggcaccctatgtcgtcactggcagcgtagatcaaacagtaaaagtgtgggagtgccgttgattgtgtctccttcggcccctcctccctcttttcctctggatgcactctgatgataccatggttaccccattgagctctgtttaaataaatattgtcctttcatgtaaattattctggatgtagattgagcttattaaatgttacacacaaagtattcatgcatggtgaatccaaattgtatactgtaaatttacatacgttgtctagaagtaccatagggtttaaaaacctgggctggcattggtcacaccaggcctaagaaggcagaagttgaatcaattgaactagggcactaaactgaatagttgacagtgtcattttatgttggattattaattcctgtttttctttctgctatctgttggtgcctgacttgatggcctcatttggggaaaagtggtggttattagggcttttcctgaaatgtgtatctatgtaacatcacttaagtgtgcttaataaatctcctgtaaggattttagatgataaggctacaattcagaatcttctgaaccatctatgtaatgaatggggattatacattggaatttttgtcatgacacatttgccaaatcagtaggatatatttgttttggcagcctatcacgcagaggctagtggtatatttatgtaagaaaatgactgtaaatctcaagaaaaatctcagcagctaatagcaactcatttatttcattttggtcttaatgctttgtaaacaggtcaaaaaatactgtcatactctaagcttctattttccacactggacatacttctagttgtattctccatactattagactgtgtagtgatgtgacttccaagtagaatttaatctccccattgagtgtgtcatggtacaaatcactattcgtttttggtgttttttagggatgtgcaatgtgcattacataatgacagaaatactgagaaggttctgtgtgcccatttgaaaggagtgggaggaatacagcagtttgtttttcaacatgaatctgatattgatttaaactgtgtttcacttacaagttttaaaaaaatgacagggtttaatggagcgtgcataaaaatgtactgttttcaccttttgtttatatgtaaatgtttgtaagtatatgggcctatctgtaagtgggtaagtctgtatgtgtgtatcatacacatcaacctccatgtccttagtcctgggtttttgaaaaagtgctaaaacggacaagtagaataaatgttgctgtggaatgccatgctttagaacaaaccctttttgatcttaatgcttctgaaaactaggtctgactctggggatttttttccagccgaaggaaaatcacttccgttatgtccccctctaatttagccgctcgacattttacacaacccggatatgttgtatattttgacccaaagttacaggtaggtttaagagaatttttagccatgacttttggagcactattccattgtcagttattaataaagaattccattgcttagctaaccaacaggttttttttgtttccaagagagttatttgaaaagttaacagaacaatgagataacagtgacagtttaacaaagataaaattctgaactgcgttttattcatttgtgtactatgtgattttttaaatgtcccctttagtatttaatggaaaattggttcctgcaaaagacaaagggtgagagttagcgtcctgtagatacacacagagactaggccgtatattaactagaagcagctttatgtctagcttgtgtctttttgtttgtttgcttgtttgtttttagattcctgagagatgtctctggaagggaaagttttgagaactaatggctatttttgaggacaaaaattacatcttaagctaattccttaaatacatacagtaggtgaattttcaggacaatattgcctcacaaccctgcttacattgaaaagtctttttcccttagctcttctgactggatttttctacaaaactatggaaaatatctttgttcttgtttgctgctattttctgtcctattttgagaaatataaatacatagaaatggtgcatcttaacatttgtttgtacatgtataaatgtcttgtattttaattcatttttagcatgaattgtttaagggtaagccacaacatctagaaatcactcatagatattgaacaataaaggagaatggtaccgatgcaggaggaagcaagcgtgtcttcccctgcagcacacagcgacttgcgttgacaaaggaggaggaaacgattactctgtaaacaaagttatccttacttgggagattgccacagcctgctgctgagttgagttaccagacatcctccatgtgagaagcagcgaacattgaatctcagggatggcccacaactgggtccacatgtaatgagccctgtttaataacgaaggggtgggggagagcagtccgtctacaacctggaatcagatttgcaaaatttcctgcactgctgtctgacactgtcctgttgatgccctttctgactgtgttctctgttttctctgtctgctgtctaaccctgtgccttgcctgggataaggacaatgatgaggttactggtttggattgtaagtagaggacttttattaattggtttagaggttcactgctgctttgtcactttctcaatcaaattggccacttaagaaataaagagctggtagaattgcatcctcagatgattattgactgtgtgtgtgtgtgaaaacagacattccagtgccacccaaatatatatctgtaacgtgcccaagaaatcctagctgcgctcttgagagtgcatgccatggagactggtttagacaccgcgtggagcctagttgcctgttgtcacggcatcttgcactttaggagactaagaccgtcctggttcgtctgtgtgtggtgtgaccaatggtgtgcccagagcactactctcaaaatcactagtgttagcaagtcgtcccgggctggggagcgttcgccgtagtctttggaagctttggctttagatttaccaagccccgcctccccgctgccagtgccctgctctcccgttcgcctctttctgtttctgtgtgaactttcccggtaatatcactcgttaaataggttttctttaaacttaattaaggaaaaactatttaaaggtaaaggatattttgttgacatcggtggctcgatcatccttaagcaactgaagttaaaattgttgaaggaaaaggcacttaaattggttactttcatgtccagctgtatataagtccagtgtgttcatctagatgacgcaaagaatctcctggtagagaagcgacatgtaaaaaactggtggaaaaaggttttggattttttttccagtggggtggggggagggcaagctggatttacaggtcacggctggactgaatgggcctttttatcttcccactgtatcatggaagtagctgcttgcttgtactgtccatccttcaggcatccctaaagctcactctgaagatgttagagacaaacacaaactcttcgagttaaagttgatcctgacactgacatgaaggcaagccttgatttcgtatgaacgttgctgaagtggtaattgaggaaaacagttccccagattgttaagagttcactgaagatattgacacaattttaaaaaatcagtaaaggaatgtatataatattgctctcgtgttttacagtaagatttgttgctctcagactgtgtaaaacaaaatttattcatgttttctgcatattaaaaaatcttattgtaccaactggtaaaccg

SEQ ID NO: 30 - DNA, 4912 bp, Homo sapiens
  gene (1)...(4912): SOX4; SRY (sex determining region Y)-box 4
attggggtctgctctaagctgcagcaagagaaactgtgtgtgaggggaagaggcctgtttcgctgtcgggtctctagttcttgcacgctctttaagagtctgcactggaggaactcctgccattaccagctcccttcttgcagaagggagggggaaacatacatttattcatgccagtctgttgcatgcaggctttttggcttcctaccttgcaacaaaataattgcaccaactccttagtgccgattccgcccacagagagtcctggagccacagtcttttttgctttgcattgtaggagagggactaagtgctagagactatgtcgctttcctgagctaccgagagcgctcgtgaactggaatcaactgcttcagggaaaaagaaaaaaaaaaaaaaaagacttgcctgggaggccgcgagaaacttgcattggaagcttcagcaaccagcattcgagaaactcctctctactttagcacggtctccagactcagccgagagacagcaaactgcagcgcggtgagagagcgagagagagggagagagagactctccagcctgggaactataactcctctgcgagaggcggagaactccttccccaaatcttttggggacttttctctctttacccacctccgcccctgcgaggagttgaggggccagttcggccgccgcgcgcgtcttcccgttcggcgtgtgcttggcccggggaaccgggagggcccggcgatcgcgcggcggccgccgcgagggtgtgagcgcgcgtgggcgcccgccgagccgaggccatggtgcagcaaaccaacaatgccgagaacacggaagcgctgctggccggcgagagctcggactcgggcgccggcctcgagctgggaatcgcctcctcccccacgcccggctccaccgcctccacgggcggcaaggccgacgacccgagctggtgcaagaccccgagtgggcacatcaagcgacccatgaacgccttcatggtgtggtcgcagatcgagcggcgcaagatcatggagcagtcgcccgacatgcacaacgccgagatctccaagcggctgggcaaacgctggaagctgctcaaagacagcgacaagatccctttcattcgagaggcggagcggctgcgcctcaagcacatggctgactaccccgactacaagtaccggcccaggaagaaggtgaagtccggcaacgccaactccagctcctcggccgccgcctcctccaagccgggggagaagggagacaaggtcggtggcagtggcgggggcggccatgggggcggcggcggcggcgggagcagcaacgcggggggaggaggcggcggtgcgagtggcggcggcgccaactccaaaccggcgcagaaaaagagctgcggctccaaagtggcgggcggcgcgggcggtggggttagcaaaccgcacgccaagctcatcctggcaggcggcggcggcggcgggaaagcagcggctgccgccgccgcctccttcgccgccgaacaggcgggggccgccgccctgctgcccctgggcgccgccgccgaccaccactcgctgtacaaggcgcggactcccagcgcctcggcctccgcctcctcggcagcctcggcctccgcagcgctcgcggccccgggcaagcacctggcggagaagaaggtgaagcgcgtctacctgttcggcggcctgggcacgtcgtcgtcgcccgtgggcggcgtgggcgcgggagccgaccccagcgaccccctgggcctgtacgaggaggagggcgcgggctgctcgcccgacgcgcccagcctgagcggccgcagcagcgccgcctcgtcccccgccgccggccgctcgcccgccgaccaccgcggctacgccagcctgcgcgccgcctcgcccgccccgtccagcgcgccctcgcacgcgtcctcctcggcctcgtcccactcctcctcttcctcctcctcgggctcctcgtcctccgacgacgagttcgaagacgacctgctcgacctgaaccccagctcaaactttgagagcatgtccctgggcagcttcagttcgtcgtcggcgctcgaccgggacctggattttaacttcgagcccggctccggctcgcacttcgagttcccggactactgcacgcccgaggtgagcgagatgatctcgggagactggctcgagtccagcatctccaacctggttttcacctactgaagggcgcgcaggcagggagaagggccggggggggtaggagaggagaaaaaaaaagtgaaaaaaagaaacgaaaaggacagacgaagagtttaaagagaaaagggaaaaaagaaagaaaaagtaagcagggctggcttcgcccgcgttctcgtcgtcggatcaaggagcgcggcggcgttttggacccgcgctcccatcccccaccttcccgggccggggacccactctgcccagccggagggacgcggaggaggaagagggtagacaggggcgacctgtgattgttgttattgatgttgttgttgatggcaaaaaaaaaaaagcgacttcgagtttgctcccctttgcttgaagagaccccctcccccttccaacgagcttccggacttgtctgcacccccagcaagaaggcgagttagttttctagagacttgaaggagtctcccccttcctgcatcaccaccttggttttgttttattttgcttcttggtcaagaaaggaggggagaacccagcgcacccctccccccctttttttaaacgcgtgatgaagacagaaggctccggggtgacgaatttggccgatggcagatgttttgggggaacgccgggactgagagactccacgcaggcgaattcccgtttggggcttttttttcctccctcttttccccttgccccctctgcagccggaggaggagatgttgaggggaggaggccagccagtgtgaccggcgctaggaaatgacccgagaaccccgttggaagcgcagcagcgggagctaggggcgggggcggaggaggacacgaactggaagggggttcacggtcaaactgaaatggatttgcacgttggggagctggcggcggcggctgctgggcctccgccttcttttctacgtgaaatcagtgaggtgagacttcccagaccccggaggcgtggaggagaggagactgtttgatgtggtacaggggcagtcagtggagggcgagtggtttcggaaaaaaaaaaagaaaaaaagaaaaaaaaagaaaaaaaaaagatttttttcttctcttaatcggaatcgtgatggtgttggattatttcaatggtggggttaatatagcatgttatcctgtctatcttttaaagatttctgtataagactgttgagcagtttttaaaatagtgtaggataatataaaaagcagatagatggcgctatgtttgattcctacaacgaaattatcaccagctttttttcattcttaactctttaaaggattcaaacgcaactcaaatctgtgctggactttaaaaaaacaattcaggaccaaattttttctcagtgtgtgtgtttattccttataggtgtaaatgagaagacgtgtttttttccttcaccgatgctccatcctcgtatttctttttccttgtaaatgtaatcagatgccattttatatgtggacgtatttatactggccaaacatattttttcttttgtccctttttttctttcctttctttttacttcctttatttctttattccttccttttcctttttttcttttttttttctttttttttttttttttttggtagttgttgttacccacgccattttacgtctccttcactgaagggctagagttttaacttttaattttttatatttaaatgtagacttttgacacttttaaaaaacaaaaaaagacaagagagatgaaaacgtttgattattttctcagtgtatttttgtaaaaaatatataaagggggtgttaatcggtgtaaatcgctgtttggatttcctgattttataacagggcggctggttaatatctcacacagtttaaaaaatcagcccctaatttctccatgtttacacttcaatctgcaggcttcttaaagtgacagtatcccttaacctgccaccagtgtccaccctccggcccccgtcttgtaaaaaggggaggagaattagccaaacactgtaagcttttaagaaaaacaaagttttaaacgaaatactgctctgtccagaggctttaaaactggtgcaattacagcaaaaagggattctgtagctttaacttgtaaaccacatcttttttgcactttttttataagcaaaaacgtgccgtttaaaccactggatctatctaaatgccgatttgagttcgcgacactatgtactgcgtttttcattcttgtatttgactatttaatcctttctacttgtcgctaaatataattgttttagtcttatggcatgatgatagcatatgtgttcaggtttatagctgttgtgtttaaaaattgaaaaaagtggaaaacatctttgtacatttaagtctgtattataataagcaaaaagattgtgtgtatgtatgtttaatataacatgacaggcactaggacgtctgcctttttaaggcagttccgttaagggtttttgtttttaaacttttttttgccatccatcctgtgcaatatgccgtgtagaatatttgtcttaaaattcaaggccacaaaaacaatgtttgggggaaaaaaaagaaaaaatcatgccagctaatcatgtcaagttcactgcctgtcagattgttgatatataccttctgtaaataactttttttgagaaggaaataaaatcagctggaactgaaccctaaaaaaaaaaaaaaaaaaaaaaaaaaaaaaaaaaaa

SEQ ID NO: 31 - DNA, 1216 bp, Homo sapiens
  gene (1)...(1216): ASNA1; arsA arsenite transporter, ATP-binding, homolog 1 (bacterial)
atgctcctcgatgtggagccgctggagcctacacttagcaacatcatcgagcagcgcagcctgaagtggatcttcgtcgggggcaagggtggtgtgggcaagaccacctgcagctgcagcctggcagtccagctctccaaggggcgtgagagtgttctgatcatctccacagacccagcacacaacatctcagatgcttttgaccagaagttctcaaaggtgcctaccaaggtcaaaggctatgacaacctctttgctatggagattgaccccagcctgggcgtggcggacgtgcctgacgagttcttcgaggaggacaacatgctgagcatgggcaagaagatgatgcaggaggccatgagcgcatttcccggcatcgatgaggccatgagctatgccgaggtcatgaggctggtgaagggcatgaacttctcggtggtggtatttgacacggcacccacgggccacaccctgaggctgctcaacttccccaccatcgtggagcggggcctgggccggcttatgcagatcaagaaccagatcagccctttcatctcacagatgtgcaacatgctgggcctgggggacatgaacgcagaccagctggcctccaagctggaggagacgctgcccgtcatccgctcagtcagcgaacagttcaaggaccctgagcagacaactttcatctgcgtatgcattgctgagttcctgtccctgtatgagacagagaggctgatccaggagctggccaagtgcaagattgacacacacaatataattgtcaaccagctcgtcttccccgaccccgagaagccctgcaagatgtgtgaggcccgtcacaagatccaggccaagtatctggaccagatggaggacctgtatgaagacttccacatcgtgaagctgccgctgttaccccatgaggtgcggggggcagacaaggtcaacaccttctcggccctcctcctggagccctacaagccccccagtgcccagtagcacagctgccagccccaaccgctgccatttcacactcaccctccaccctccccaccccctcggggcagagtttgcacaaagtcccccccataatacagggggagccacttgggcaggaggcagggaggggtccattccccctggtggggctggtggggagctgtagttgccccctacctctcccacctcttgctcttcaataaatgatcttaaactg

SEQ ID NO: 32 - DNA, 4917 bp, Homo sapiens
  gene (1)...(4917): MAN2A2; mannosidase, alpha, class 2A, member 2
ggcagctcggccgactgggcccggagcggcgcggaggccgggcgctgacggtgtgtgtggaggccagtatgaagctgaaaaagcaggtgacagtgtgtggggctgccatcttctgtgtggcagtcttctcgctctacctcatgctggaccgagtgcaacacgatcccacccgacaccagaatggtgggaacttcccccggagccaaatttctgtgctgcagaaccgcattgagcagctggagcagcttttggaggagaaccatgagattatcagccatatcaaggactccgtgctggagctgacagccaacgcagagggcccgcccgccatgctgccctactacacggtcaatggctcctgggtggtgccaccggagccccggcccagcttcttctccatctccccgcaggactgccagtttgctttggggggccggggtcagaagccagagctgcagatgctcactgtgtcggaggagctgccgtttgacaacgtggatggtggtgtgtggaggcaaggcttcgacatctcctacgacccgcacgactgggatgctgaagacctgcaggtgtttgtggtgccccactctcacaatgacccaggctggatcaagacctttgacaagtactacacagagcagacccaacacatcctcaatagcatggtgtctaagctgcaggaggacccccggcggcgcttcctctgggcagaggtctccttcttcgccaagtggtgggacaacatcaatgtccaaaagagagcggcagtccgaaggctggtgggaaacgggcagctggagattgcgacaggaggctgggtgatgccagatgaggccaattcccactactttgcattgattgaccagctcatcgaaggacaccagtggctggagagaaatcttggtgcaaccccccgctctggctgggcagtggacccctttggatacagctccaccatgccttacctgctgcgccgtgccaacctcaccagcatgctgattcagagagtgcactatgccatcaagaagcactttgctgccacccacagcctagagttcatgtggaggcagacatgggactcggactccagcacagacatcttctgtcacatgatgcccttctacagctatgacgtcccccatacctgtggcccagatcccaagatctgctgccaatttgatttcaaacgcctgcctggtgggcgcatcaactgcccttggaaggtgccaccccgggccatcacagaggccaacgtggcagagagggcagccctgcttctggaccaataccggaagaagtcccagctgttccgaagcaacgtcctcctggtgcctcttggagatgacttccgatatgacaagccccaggagtgggatgcccagttcttcaactaccaacggctctttgacttcttcaacagcaggcctaacctccatgtgcaggcccagtttggcactctttctgactattttgatgccctgtacaagaggacaggggtggagccaggggcccggcctccagggtttcctgtgctgagcggggatttcttctcctatgcggaccgggaggatcattactggacaggctattacacttcccggcccttctacaagagcttagaccgagtcctggaagcccacctgcggggggcagaggttctgtacagcctggctgcagctcacgctcgccgctctggtctggctggccggtacccactgtctgatttcaccctcctgacggaagctcggcgcacattggggctcttccagcatcacgatgccatcactggcacggccaaggaggctgtggtggtggactatggggtcaggcttctgcgctcccttgtcaacctgaagcaggtcatcattcatgcagcccactatctggtgctgggggacaaggagacctaccactttgaccctgaggcgcccttcctccaagtggatgacactcgcttaagtcacgacgccctcccagagcgcacggtgatccagctggattcctcgcccaggtttgtggtcctattcaacccactggaacaggagcgattcagcatggtgtccctgctggtcaactctccccgcgtgcgtgtcctttcggaggagggtcagcccctggccgtgcagatcagcgcacactggagctctgccaccgaggcggtccctgacgtctaccaggtgtctgtgcctgtccgcctgccagccctgggcctgggcgtgctgcagctacagctgggcctggatgggcaccgcacgctgccctcctctgtgcgcatctacctgcacggccggcagctgtccgtcagcaggcacgaagcgtttcctctccgtgtcattgactctggcaccagcgacttcgccctcagcaaccgctacatgcaggtctggttctcaggccttactgggctcctcaaggggtcagggctgtgttttttggcagagcatccgaagggtggatgaggagcacgagcagcaggtggacatgcaggtccttgtctatggcacccgtacgtccaaagacaagagtggagcctacctcttcctgcccgatggcgaggcaagccctacgtccccaaggagccccccgtgctgcgtgtcactgaaggccctttcttctcagaggtggttgcgtactatgagcacattcaccaggcggtccggctttacaatctgccaggggtggaggggctgtctctggacatatcatccctggtggacatccgggactacgtcaacaaggagctggccctgcacatccatacagacatcgacagccagggtgcagccccgacggtatctgaagaagctccccctccaggccaacttctaccccatgccagtcatggcctatatccaggacgcacagaagcgcctcacgctgcacactgcccaggccctgggtgtctctagcctcaaagatggccagctggaggtgatcttggaccggcggctgatgcaggatgacaaccggggcctaggccaagggctcaaggacaacaagagaacctgcaaccgtttccgcctcctgctagagcggcgaaccgtgggcagtgaggtccaagatagccactctaccagctacccatccctcctcagccacctgacctccatgtacctgaacgccccggcgctcgctctgcctgtagccaggatgcagctcccaggccctggtctgcgctcatttcatcctctggcttcctcactgccctgtgacttccacctgctcaacctacgtacgctccaggctgaggaggacaccctaccctcggcggagaccgcactcatcttacaccgcaagggttttgactgcggcctggaggccaagaacttgggcttcaactgcaccacaagccaaggcaaggtagccctgggcagccttttccatggcctggatgtggtattccttcagccaacctccttgacgttactgtaccctctggcctccccgtccaacagcactgacgtctatttggagcccatggagattgctacctttcgcctccgcttgggttagggcttcttgtggcctgaagagaaagttcattcacagagactgcctcttaacatgaagatcattggacaagccacacgggtatcccatcccgatctgcctcccagaactgtgacacactgggctctgccctcattttctgtttattgctgctgctgtgttttcggcgcaacccacaaacccagtgatgggtaaatagggcagacgccagtgagatcagggagagaaggcccttggtcagagtgggcagtgccaggctctgctttgggttgtgagtggacacccaactgggcacaggctcaggcacccatcctttttccaaacagggatatagaagtggtggaagcagacagaagaggtaagggaggctaagtgggtaacagcccagcatcagggtcactgtggcaacagcaggctctaggggaatcctgtggttatgtagagactccatgtcctggtgtgatgagcaggatcagagtgactctgggaggacaggggtggggacccagagttagcagtggggatggagcagtagaaggaatcactgtttctcctaggagtctgaaggcctcgctgctttctgtgatggctttgcagtaagtgccgcctggcctgcatgcattggctaacaggctgcagaatggcaggaaggactcgctagagattgtcatggccagagatcataggtcacttcaggtagcaagacccctggcaaactgggcacttggcctatgtactgatttgtgggatggtggcaggggtgtggggtccttcaccctgcctgaattctctttggcttctgtgctctgtatgctgctgtccccaagggctctttcttattatggcagggagtggggattggtcctactttctttctctggaaaggaaagcctccaagactccatgtgcttgggcagcttgagaaggcgttcagcaccacgcctagcaggcagaccttgaagcctcacctttagtctatctgcagaggtattcagttcctggcacaggggactaggggcatgtagagtatatgaggaggcagtatggctgtgcaggagccttcatttcagcttcaattaatagggaagaatttatgatagctctatagatgctgaaaaggtatttcgtaagatttaaaatccatcccttattaaaactcttagtaaattaagtctggaaagaaacaccctaatctagataaaggtctgtttcagaaaccaacagtgatggcattctaaagagtcagacgccacaggcattcccattaaagtcagaaactagccaagggcaagctattattcagcagtgtcccggcactactaacccctgcaacaagccagatgaggaacataaggaagaattataattgtcattatttgtagacaataaaactgcctacctgtaaaa

SEQ ID NO: 33 - DNA, 1965 bp, Homo sapiens
  gene (1)...(1965): NFYC; nuclear transcription factor Y, gamma
acgcgtccggggaaacggtgcaaacggcgtggccgccatcttgcttgtgcccccgcttcgcgcgcgctccgtgacgcacacttcccccctcccctccgccgcgcctgggcctctgcattgcccgactccgtaggagcgcgggggcggctcctgctcttcctggactcctgagcagagttgtcgagatgtccacagaaggaggatttggtggtactagcagcagtgatgcccagcaaagcctacagtcgttctggcctcgggtcatggaagaaatccggaatttaacagtgaaagacttccgagtgcaggaactcccactggctcgtattaagaagattatgaaactggatgaagatgtgaagatgatcagtgcagaagcgcctgtactctttgccaaggcagcccagatttttatcacagagttgactcttcgagcctggattcacacagaagataacaagcgccggactctacagagaaatgatatcgccatggcaattacaaaatttgatcagtttgattttctcatcgatattgttccaagagatgaactgaaacctccaaagcgtcaggaggaggtgcgccagtctgtaactcctgccgagccagtccagtactatttcacgctggctcagcaacccaccgctgtccaagtccagggccagcagcaaggccagcagaccaccagctccacgaccaccatccagcctgggcagatcatcatcgcacagcctcagcagggccagaccacacctgtgacaatgcaggttggagaaggtcagcaggtgcagattgtccaggctcagccacagggtcaagcccaacaggcccagagtggcactggacagaccatgcaggtgatgcagcagatcatcactaacacaggagagatccagcagatcccggtgcagctgaatgccggccagctgcagtatatccgcttagcccagcctgtatcaggcactcaagttgtgcagggacagatccagacacttgccaccaatgctcaacagattacacagacagaggtccagcaaggacagcagcagttcagccagttcacagatggacagcagctctaccagatccagcaagtcaccatgcctgcgggccaggacctcgcccagcccatgttcatccagtcagccaaccagccctccgacgggcaggccccccaggtgaccggcgactgagggcctgagctggcaaggccaaggacacccaacacaatttttgccatacagccccaggcaatgggcacagccttcctccccagaggacccggccgacctcagcgcctcctgcaggctaggacactggtgcactacaccccatgcctgggggccgagattctccagcagaaagatgcaatattttttgtttcctttttttccatttttttctctaaggaatcaatatttcaatatgttgagtgtgtgtccaatgctatgaaattaaaatattaaataacatatttatggcattttcttgaagagtgtggttgaagaaatatttctccttttgtttttcttttttttttgtttgttactgccacttctttttaggagcaaatctccccaggggtgtacggtatttcttgactctgggaacagctgctacccccaagacttgccacgttgttctgccctcagatggaattaggtgaatgtgtgtagctgctttttcactcgtggtcctctccctatcccttgctctgaccccagagctctgtgtatttgcatccagaggccatggaaacattctttgcatttaagagacagatttatccctgtggagagtgggtggattcattgccacactcttttctcccagggacccaggaaactaggactttgtgtgtttgctgcccacctcccttttattttttaaatgcattaaaaactgtgctagtctcctttgcatggacttcaagctgcatgaaatgcaataaatctcattttagat

SEQ ID NO: 34 - DNA, 11433 bp, Homo sapiens
  gene (1)...(11433): NOTCH2; Notch homolog 2
aggctgcttcgttgcacacccgagaaagtttcagccaaacttcgggcggcggctgaggcggcggccgaggagcggcggactcggggcgcggggagtcgaggcatttgcgcctgggcttcggagcgtagcgccagggcctgagcctttgaagcaggaggaggggaggagagagtggggctcctctatcgggaccccctccccatgtggatctgcccaggcggcggcggcggcggcggaggaggaggcgaccgagaagatgcccgccctgcgccccgctctgctgtgggcgctgctggcgctctggctgtgctgcgcggcccccgcgcatgcattgcagtgtcgagatggctatgaaccctgtgtaaatgaaggaatgtgtgttacctaccacaatggcacaggatactgcaaatgtccagaaggcttcttgggggaatattgtcaacatcgagacccctgtgagaagaaccgctgccagaatggtgggacttgtgtggcccaggccatgctggggaaagccacgtgccgatgtgcctcagggtttacaggagaggactgccagtactcaacatctcatccatgctttgtgtctcgaccctgcctgaatggcggcacatgccatatgctcagccgggatacctatgagtgcacctgtcaagtcgggtttacaggtaaggagtgccaatggacggatgcctgcctgtctcatccctgtgcaaatggaagtacctgtaccactgtggccaaccagttctcctgcaaatgcctcacaggcttcacagggcagaaatgtgagactgatgtcaatgagtgtgacattccaggacactgccagcatggtggcacctgcctcaacctgcctggttcctaccagtgccagtgccctcagggcttcacaggccagtactgtgacagcctgtatgtgccctgtgcaccctcaccttgtgtcaatggaggcacctgtcggcagactggtgacttcacttttgagtgcaactgccttccaggttttgaagggagcacctgtgagaggaatattgatgactgccctaaccacaggtgtcagaatggaggggtttgtgtggatggggtcaacacttacaactgccgctgtcccccacaatggacaggacagttctgcacagaggatgtggatgaatgcctgctgcagcccaatgcctgtcaaaatgggggcacctgtgccaaccgcaatggaggctatggctgtgtatgtgtcaacggctggagtggagatgactgcagtgagaacattgatgattgtgccttcgcctcctgtactccaggctccacctgcatcgaccgtgtggcctccttctcttgcatgtgcccagaggggaaggcaggtctcctgtgtcatctggatgatgcatgcatcagcaatccttgccacaagggggcactgtgtgacaccaaccccctaaatgggcaatatatttgcacctgcccacaaggctacaaaggggctgactgcacagaagatgtggatgaatgtgccatggccaatagcaatccttgtgagcatgcaggaaaatgtgtgaacacggatggcgccttccactgtgagtgtctgaagggttatgcaggacctcgttgtgagatggacatcaatgagtgccattcagacccctgccagaatgatgctacctgtctggataagattggaggcttcacatgtctgtgcatgccaggtttcaaaggtgtgcattgtgaattagaaataaatgaatgtcagagcaacccttgtgtgaacaatgggcagtgtgtggataaagtcaatcgtttccagtgcctgtgtcctcctggtttcactgggccagtttgccagattgatattgatgactgttccagtactccgtgtctgaatggggcaaagtgtatcgatcacccgaatggctatgaatgccagtgtgccacaggtttcactggtgtgttgtgtgaggagaacattgacaactgtgaccccgatccttgccaccatggtcagtgtcaggatggtattgattcctacacctgcatctgcaatcccgggtacatgggcgccatctgcagtgaccagattgatgaatgttacagcagcccttgcctgaacgatggtcgctgcattgacctggtcaatggctaccagtgcaactgccagccaggcacgtcaggggttaattgtgaaattaattttgatgactgtgcaagtaacccttgtatccatggaatctgtatggatggcattaatcgctacagttgtgtctgctcaccaggattcacagggcagagatgtaacattgacattgatgagtgtgcctccaatccctgtcgcaagggtgcaacatgtatcaacggtgtgaatggtttccgctgtatatgccccgagggaccccatcaccccagctgctactcacaggtgaacgaatgcctgagcaatccctgcatccatggaaactgtactggaggtctcagtggatataagtgtctctgtgatgcaggctgggttggcatcaactgtgaagtggacaaaaatgaatgcctttcgaatccatgccagaatggaggaacttgtgacaatctggtgaatggatacaggtgtacttgcaagaagggctttaaaggctataactgccaggtgaatattgatgaatgtgcctcaaatccatgcctgaaccaaggaacctgctttgatgacataagtggctacacttgccactgtgtgctgccatacacaggcaagaattgtcagacagtattggctccctgttccccaaacccttgtgagaatgctgctgtttgcaaagagtcaccaaattttgagagttatacttgcttgtgtgctcctggctggcaaggtcagcggtgtaccattgacattgacgagtgtatctccaagccctgcatgaaccatggtctctgccataacacccagggcagctacatgtgtgaatgtccaccaggcttcagtggtatggactgtgaggaggacattgatgactgccttgccaatccttgccagaatggaggttcctgtatggatggagtgaatactttctcctgcctctgccttccgggtttcactggggataagtgccagacagacatgaatgagtgtctgagtgaaccctgtaagaatggagggacctgctctgactacgtcaacagttacacttgcaagtgccaggcaggatttgatggagtccattgtgagaacaacatcaatgagtgcactgagagctcctgtttcaatggtggcacatgtgttgatgggattaactccttctcttgcttgtgccctgtgggtttcactggatccttctgcctccatgagatcaatgaatgcagctctcatccatgcctgaatgagggaacgtgtgttgatggcctgggtacctaccgctgcagctgccccctgggctacactgggaaaaactgtcagaccctggtgaatctctgcagtcggtctccatgtaaaaacaaaggtacttgcgttcagaaaaaagcagagtcccagtgcctatgtccatctggatgggctggtgcctattgtgacgtgcccaatgtctcttgtgacatagcagcctccaggagaggtgtgcttgttgaacacttgtgccagcactcaggtgtctgcatcaatgctggcaacacgcattactgtcagtgccccctgggctatactgggagctactgtgaggagcaactcgatgagtgtgcgtccaacccctgccagcacggggcaacatgcagtgacttcattggtggatacagatgcgagtgtgtcccaggctatcagggtgtcaactgtgagtatgaagtggatgagtgccagaatcagccctgccagaatggaggcacctgtattgaccttgtgaaccatttcaagtgctcttgcccaccaggcactcggggcctactctgtgaagagaacattgatgactgtgcccggggtccccattgccttaatggtggtcagtgcatggataggattggaggctacagttgtcgctgcttgcctggctttgctggggagcgttgtgagggagacatcaacgagtgcctctccaacccctgcagctctgagggcagcctggactgtatacagctcaccaatgactacctgtgtgtttgccgtagtgcctttactggccggcactgtgaaaccttcgtcgatgtgtgtccccagatgccctgcctgaatggagggacttgtgctgtggccagtaacatgcctgatggtttcatttgccgttgtcccccgggattttccggggcaaggtgccagagcagctgtggacaagtgaaatgtaggaagggggagcagtgtgtgcacaccgcctctggaccccgctgcttctgccccagtccccgggactgcgagtcaggctgtgccagtagcccctgccagcacgggggcagctgccaccctcagcgccagcctccttattactcctgccagtgtgccccaccattctcgggtagccgctgtgaactctacacggcaccccccagcacccctcctgccacctgtctgagccagtattgtgccgacaaagctcgggatggcgtctgtgatgaggcctgcaacagccatgcctgccagtgggatgggggtgactgttctctcaccatggagaacccctgggccaactgctcctccccacttccctgctgggattatatcaacaaccagtgtgatgagctgtgcaacacggtcgagtgcctgtttgacaactttgaatgccaggggaacagcaagacatgcaagtatgacaaatactgtgcagaccacttcaaagacaaccactgtgaccaggggtgcaacagtgaggagtgtggttgggatgggctggactgtgctgctgaccaacctgagaacctggcagaaggtaccctggttattgtggtattgatgccacctgaacaactgctccaggatgctcgcagcttcttgcgggcactgggtaccctgctccacaccaacctgcgcattaagcgggactcccagggggaactcatggtgtacccctattatggtgagaagtcagctgctatgaagaaacagaggatgacacgcagatcccttcctggtgaacaagaacaggaggtggctggctctaaagtctttctggaaattgacaaccgccagtgtgttcaagactcagaccactgcttcaagaacacggatgcagcagcagctctcctggcctctcacgccatacaggggaccctgtcataccctcttgtgtctgtcgtcagtgaatccctgactccagaacgcactcagctcctctatctccttgctgttgctgttgtcatcattctgtttattattctgctgggggtaatcatggcaaaacgaaagcgtaagcatggctctctctggctgcctgaaggtttcactcttcgccgagatgcaagcaatcacaagcgtcgtgagccagtgggacaggatgctgtggggctgaaaaatctctcagtgcaagtctcagaagctaacctaattggtactggaacaagtgaacactgggtcgatgatgaagggccccagccaaagaaagtaaaggctgaagatgaggccttactctcagaagaagatgaccccattgatcgacggccatggacacagcagcaccttgaagctgcagacatccgtaggacaccatcgctggctctcacccctcctcaggcagagcaggaggtggatgtgttagatgtgaatgtccgtggcccagatggctgcaccccattgatgttggcttctctccgaggaggcagctcagatttgagtgatgaagatgaagatgcagaggactcttctgctaacatcatcacagacttggtctaccagggtgccagcctccaggcccagacagaccggactggtgagatggccctgcaccttgcagcccgctactcacgggctgatgctgccaagcgtctcctggatgcaggtgcagatgccaatgcccaggacaacatgggccgctgtccactccatgctgcagtggcagctgatgcccaaggtgtcttccagattctgattcgcaaccgagtaactgatctagatgccaggatgaatgatggtactacacccctgatcctggctgcccgcctggctgtggagggaatggtggcagaactgatcaactgccaagcggatgtgaatgcagtggatgaccatggaaaatctgctcttcactgggcagctgctgtcaataatgtggaggcaactcttttgttgttgaaaaatggggccaaccgagacatgcaggacaacaaggaagagacacctctgtttcttgctgcccgggaggggagctatgaagcagccaagatcctgttagaccattttgccaatcgagacatcacagaccatatggatcgtcttccccgggatgtggctcgggatcgcatgcaccatgacattgtgcgccttctggatgaatacaatgtgaccccaagccctccaggcaccgtgttgacttctgctctctcacctgtcatctgtgggcccaacagatctttcctcagcctgaagcacaccccaatgggcaagaagtctagacggcccagtgccaagagtaccatgcctactagcctccctaaccttgccaaggaggcaaaggatgccaagggtagtaggaggaagaagtctctgagtgagaaggtccaactgtctgagagttcagtaactttatcccctgttgattccctagaatctcctcacacgtatgtttccgacaccacatcctctccaatgattacatcccctgggatcttacaggcctcacccaaccctatgttggccactgccgcccctcctgccccagtccatgcccagcatgcactatctttttctaaccttcatgaaatgcagcctttggcacatggggccagcactgtgcttccctcagtgagccagttgctatcccaccaccacattgtgtctccaggcagtggcagtgctggaagcttgagtaggctccatccagtcccagtcccagcagattggatgaaccgcatggaggtgaatgagacccagtacaatgagatgtttggtatggtcctggctccagctgagggcacccatcctggcatagctccccagagcaggccacctgaagggaagcacataaccacccctcgggagcccttgccccccattgtgactttccagctcatccctaaaggcagtattgcccaaccagcgggggctccccagcctcagtccacctgccctccagctgttgcgggccccctgcccaccatgtaccagattccagaaatggcccgtttgcccagtgtggctttccccactgccatgatgccccagcaggacgggcaggtagctcagaccattctcccagcctatcatcctttcccagcctctgtgggcaagtaccccacacccccttcacagcacagttatgcttcctcaaatgctgctgagcgaacacccagtcacagtggtcacctccagggtgagcatccctacctgacaccatccccagagtctcctgaccagtggtcaagttcatcaccccactctgcttctgactggtcagatgtgaccaccagccctacccctgggggtgctggaggaggtcagcggggacctgggacacacatgtctgagccaccacacaacaacatgcaggtttatgcgtgagagagtccacctccagtgtagagacataactgacttttgtaaatgctgctgaggaacaaatgaaggtcatccgggagagaaatgaagaaatctctggagccagcttctagaggtaggaaagagaagatgttcttattcagataatgcaagagaagcaattcgtcagtttcactgggtatctgcaaggcttattgattattctaatctaataagacaagtttgtggaaatgcaagatgaatacaagccttgggtccatgtttactctcttctatttggagaataagatggatgcttattgaagcccagacattcttgcagcttggactgcattttaagccctgcaggcttctgccatatccatgagaagattctacactagcgtcctgttgggaattatgccctggaattctgcctgaattgacctacgcatctcctcctccttggacattcttttgtcttcatttggtgcttttggttttgcacctctccgtgattgtagccctaccagcatgttatagggcaagacctttgtgcttttgatcattctggcccatgaaagcaactttggtctcctttcccctcctgtcttcccggtatcccttggagtctcacaaggtttactttggtatggttctcagcacaaacctttcaagtatgttgtttctttggaaaatggacatactgtattgtgttctcctgcatatatcattcctggagagagaaggggagaagaatacttttcttcaacaaattttgggggcaggagatcccttcaagaggctgcaccttaatttttcttgtctgtgtgcaggtcttcatataaactttaccaggaagaagggtgtgagtttgttgtttttctgtgtatgggcctggtcagtgtaaagttttatccttgatagtctagttactatgaccctccccacttttttaaaaccagaaaaaggtttggaatgttggaatgaccaagagacaagttaactcgtgcaagagccagttacccacccacaggtccccctacttcctgccaagcattccattgactgcctgtatggaacacatttgtcccagatctgagcattctaggcctgtttcactcactcacccagcatatgaaactagtcttaactgttgagcctttcctttcatatccacagaagacactgtctcaaatgttgtacccttgccatttaggactgaactttccttagcccaagggacccagtgacagttgtcttccgtttgtcagatgatcagtctctactgattatcttgctgcttaaaggcctgctcaccaatctttctttcacaccgtgtggtccgtgttactggtatacccagtatgttctcactgaagacatggactttatatgttcaagtgcaggaattggaaagttggacttgttttctatgatccaaaacagccctataagaaggttggaaaaggaggaactatatagcagcctttgctattttctgctaccatttcttttcctctgaagcggccatgacattccctttggcaactaacgtagaaactcaacagaacattttcctttcctagagtcaccttttagatgataatggacaactatagacttgctcattgttcagactgattgcccctcacctgaatccactctctgtattcatgctcttggcaatttctttgactttcttttaagggcagaagcattttagttaattgtagataaagaatagttttcttcctcttctccttgggccagttaataattggtccatggctacactgcaacttccgtccagtgctgtgatgcccatgacacctgcaaaataagttctgcctgggcattttgtagatattaacaggtgaattcccgactcttttggtttgaatgacagttctcattccttctatggctgcaagtatgcatcagtgcttcccacttacctgatttgtctgtcggtggccccatatggaaaccctgcgtgtctgttggcataatagtttacaaatggttttttcagtcctatccaaatttattgaaccaacaaaaataattacttctgccctgagataagcagattaagtttgttcattctctgctttattctctccatgtggcaacattctgtcagcctctttcatagtgtgcaaacattttatcattctaaatggtgactctctgcccttggacccatttattattcacagatggggagaacctatctgcatggacctctgtggaccacagcgtacctgcccctttctgccctcctgctccagccccacttctgaaagtatcagctactgatccagccactggatattttatatcctcccttttccttaagcacaatgtcagaccaaattgcttgtttctttttcttggactactttaatttggatcctttgggtttggagaaagggaatgtgaaagctgtcattacagacaacaggtttcagtgatgaggaggacaacactgcctttcaaactttttactgatctcttagattttaagaactcttgaattgtgtggtatctaataaaagggaaggtaagatggataatcactttctcatttgggttctgaattggagactcagtttttatgagacacatcttttatgccatgtatagatcctcccctgctatttttggtttatttttattgttataaatgctttctttctttgactcctcttctgcctgcctttggggataggtttttttgtttgtttatttgcttcctctgttttgttttaagcatcattttcttatgtgaggtggggaagggaaaggtatgagggaaagagagtctgagaattaaaatattttagtataagcaattggctgtgatgctcaaatccattgcatcctcttattgaatttgccaatttgtaatttttgcataataaagaaccaaaggtgtaatgttttgttgagaggtggtttagggattttggccctaaccaatacattgaatgtatgatgactatttgggaggacacatttatgtacccagaggcccccactaataagtggtactatggttacttccttgtgtacatttctcttaaaagtgatattatatctgtttgtatgagaaacccagtaaccaataaaatgaccgcatattcctgactaaacgtagtaaggaaaatgcacactttgtttttacttttccgtttcattctaaaggtagttaagatgaaatttatatgaaagcatttttatcacaaaataaaaaaggtttgccaagctcagtggtgttgtattttttattttccaatactgcatccatggcctggcagtgttacctcatgatgtcataatttgctgagagagcaaattttcttttctttctgaatcccacaaagcctagcaccaaacttctttttttcttcctttaattagatcataaataaatgatcctggggaaaaagcatctgtcaaataggaaacatcacaaaactgagcactcttctgtgcactagccatagctggtgacaaacagatggttgctcagggacaaggtgccttccaatggaaatgcgaagtagttgctatagcaagaattgggaactgggatataagtcataatattaattatgctgttatgtaaatgattggtttgtaacattccttaagtgaaatttgtgtagaacttaatatacaggattataaaataatattttgtgtataaatttgttataagttcacattcatacatttatttataaagtcagtgagatatttgaacatgaaaaaaaaaa

SEQ ID NO: 35 - DNA, 3753 bp, Homo sapiens
  gene (1)...(3753): HDAC5; histone deacetylase 5
gcggagccggaggagccgccgcaaagatggaggagccgtcgaggaggtgctgccgccgctgccgccgccgctgctgccgccgccgcccgcgaagccggagctcgagccgcagcggggatgccgttctgagtgcctgactgcctcgccccgaaggatggcctcggatgggcattagaggcacggcggccccgggctcccgtcccgtccgtctgtctgttatcgtctgtctctcttgacatcaccgcagctccaccccctcccgtcccagcccccaacgccagcttcctgcaggcccagagccggcatgaactctcccaacgagtcggatgggatgtcaggtcgggaaccatccttggaaatcctgccgcggacttctctgcacagcatccctgtgacagtggaggtgaagccggtgctgccaagagccatgcccagttccatggggggtgggggtggaggcagccccagccctgtggagctacggggggctctggtgggctctgtggaccccacactgcgggagcagcaactgcagcaggagctcctggcgctcaagcagcagcagcagctgcagaagcagctcctgttcgctgagttccagaaacagcatgaccacctgacaaggcagcatgaggtccagctgcagaagcacctcaagcagcagcaggagatgctggcagccaagcagcagcaggagatgctggcagccaagcggcagcaggagctggagcagcagcggcagcgggagcagcagcggcaggaagagctggagaagcagcggctggagcagcagctgctcatcctgcggaacaaggagaagagcaaagagagtgccattgccagcactgaggtaaagctgaggctccaggaattcctcttgtcgaagtcaaaggagcccacaccaggcggcctcaaccattccctcccacagcaccccaaatgctggggagcccaccatgcttctttggaccagagttcccctccccagagcggcccccctgggacgcctccctcctacaaactgcctttgcctgggccctacgacagtcgagacgacttccccctccgcaaaacagcctctgaacccaacttgaaagtgcgttcaaggctaaaacagaaggtggctgagcggagaagcagtcccctcctgcgtcgcaaggatgggactgttattagcacctttaagaagagagctgttgagatcacaggtgccgggcctggggcgtcgtccgtgtgtaacagcgcacccggctccggccccagctctcccaacagctcccacagcaccatcgctgagaatggctttactggctcagtccccaacatccccactgagatgctccctcagcaccgagccctccctctggacagctcccccaaccagttcagcctctacacgtctccttctctgcccaacatctccctagggctgcaggccacggtcactgtcaccaactcacacctcactgcctccccgaagctgtcgacacagcaggaggccgagaggcaggccctccagtccctgcggcagggtggcacgctgaccggcaagttcatgagcacatcctctattcctggctgcctgctgggcgtggcactggagggcgacgggagcccccacgggcatgcctccctgctgcagcatgtgctgttgctggagcaggcccggcagcagagcaccctcattgctgtgccactccacgggcagtccccactagtgacgggtgaacgtgtggccaccagcatgcggacggtaggcaagctcccgcggcatcggcccctgagccgcactcagtcctcaccgctgccgcagagtccccaggccctgcagcagctggtcatgcaacaacagcaccagcagttcctggagaagcagaagcagcagcagctacagctgggcaagatcctcaccaagacaggggagctgcccaggcagcccaccacccaccctgaggagacagaggaggagctgacggagcagcaggaggtcttgctgggggagggagccctgaccatgccccgggagggctccacagagagtgagagcacacaggaagacctggaggaggaggacgaggaagaggatggggaggaggaggaggattgcatccaggttaaggacgaggagggcgagagtggtgctgaggaggggcccgacttggaggagcctggtgctggatacaaaaaactgttctcagatgcccagccgctgcagcctttgcaggtgtaccaggcgcccctcagcctggccactgtgccccaccaggccctgggccgtacccagtcctcccctgctgcccctgggggcatgaagagccccccagaccagcccgtcaagcacctcttcaccacaggtgtggtctacgacacgttcatgctaaagcaccagtgcatgtgcgggaacacacacgtgcaccctgagcatgctggccggatccagagcatctggtcccggctgcaggagacaggcctgcttagcaagtgcgagcggatccgaggtcgcaaagccacgctagatgagatccagacagtgcactctgaataccacaccctgctctatgggaccagtcccctcaaccggcagaagctagacagcaagaagttgctcggccccatcagccagaagatgtatgctgtgctgccttgtgggggcatcggggtggacagtgacaccgtgtggaatgagatgcactcctccagtgctgtgcgcatggcagtgggctgcctgctggagctggccttcaaggtggctgcaggagagctcaagaatggatttgccatcatccggcccccaggacaccacgccgaggaatccacagccatgggattctgcttcttcaactctgtagccatcaccgcaaaactcctacagcagaagttgaacgtgggcaaggtcctcatcgtggactgggacattcaccatggcaatggcacccagcaggcgttctataatgacccctctgtgctctacatctctctgcatcgctatgacaacgggaacttctttccaggctctggggctcctgaagaggttggtggaggaccaggcgtggggtacaatgtgaacgtggcatggacaggaggtgtggacccccccattggagacgtggagtaccttacagccttcaggacagtggtgatgcccattgcccacgagttctcacctgatgtggtcctagtctccgccgggtttgatgctgttgaaggacatctgtctcctctgggtggctactctgtcaccgccagatgttttggccacttgaccaggcagctgatgaccctggcagggggccgggtggtgctggccctggagggaggccatgacttgaccgccatctgtgatgcctctgaggcttgtgtctcggctctgctcagtgtagagctgcagcccttggatgaggcagtcttgcagcaaaagcccaacatcaacgcagtggccacgctagagaaagtcatcgagatccagagcaaacactggagctgtgtgcagaagttcgccgctggtctgggccggtccctgcgagaggcccaagcaggtgagaccgaggaggccgagactgtgagcgccatggccttgctgtcggtgggggccgagcaggcccaggctgcggcagcccgggaacacagccccaggccggcagaggagcccatggagcaggagcctgccctgtgacgccccggcccccatccctctgggcttcaccattgtgattttgtttattttttctattaaaaacaaaaagtcacacattc

SEQ ID NO: 36 - DNA, 7710 bp, Homo sapiens
  gene (1)...(7710): HCFC1; host cell factor C1 (VP16-accessory protein)
caaccaaccagtggttcatcccagccgtgaggggggacattccccctgggtgtgcagcctatggcttcgtgtgtgacgggactcgcctcctggtgtttggtgggatggtggagtatgggaaatacagcaatgacctctacgaactccaggcgagccggtgggagtggaagagactcaaagcaaagacgcccaaaaacgggccccctccgtgtcctcgactcgggcacagcttctcccttgtgggcaacaaatgctacctgtttgggggtctggccaatgatagcgaggacccaaagaacaacattccaaggtacctgaatgacttatatatcctggaattacggccaggctctggagtggtagcctgggacattcccatcacttacggggtcctaccaccaccccgggagtcacatactgccgtggtctacaccgaaaaagacaataagaagtccaagctggtgatctacggcgggatgagtggctgcaggctgggggacctgtggaccctagatattgacaccctgacgtggaataagcccagtctcagcggggtggcgcctcttcctcgcagtctccactcggcaaccaccatcggaaataaaatgtacgtgtttggtggctgggtgcctctcgtcatggatgacgtcaaagtggccacacacgagaaggagtggaagtgtaccaacacgctggcttgtctcaacctggataccatggcctgggagaccatcctgatggatacactggaggacaacatcccccgtgctcgggctggccactgcgcagtcgccatcaacacccgcctgtacatttggagtgggcgtgacggctaccgcaaggcctggaacaaccaggtctgctgcaaggacctctggtacctagagacagaaaagccaccacccccagcccgagtacaactggtacgcgccaacaccaactccctggaggtgagctggggggcagtggcaacagccgacagctaccttctccagctccagaaatatgacattcctgccacggctgctactgccacctcccctacacccaatccggtcccatctgtgcctgccaaccctcccaagagccctgccccagcagcagccgcacctgctgtgcagccgctgacccaagtaggcatcacgctcctgccccaggctgcccccgcacccccgaccaccaccaccatccaggtcttgccaacggtgcctggcagctccatttctgtgcccaccgcagccaggactcaaggtgtccctgctgttctcaaagtgaccggtcctcaggctacaacaggaactccattggtcaccatgcgacctgccagccaggctgggaaagcccctgtcaccgtgacctcccttcccgccggagtgcggatggttgtgccaacacagagtgcccagggaacggtgattggcagtagcccacagatgagtgggatggccgcactggccgctgcggccgctgccacccagaagatccccccttcctcgcgacccacggtgctgagtgtcccagcgggtaccaccatcgtgaagaccatggctgtgacacctggcactaccaccctcccagccactgtgaaggtggcctcctcgccagtcatggtgagcgtgagcaaccctgccactcgcatgctgaagactgcagccgcccaggtggggacatcggtttcctccgccaccaacacgtctacccgccctatcatcacagtgcacaagtcaggcactgtgacagtggcccagcaagcccaggtggtgaccacagttgtgggcggggtcaccaagaccatcaccctggtgaagagccccatctctgtcccaggaggcagtgctctgatttccaatctgggcaaagtgatgtcggtggtccagaccaaaccagttcagacttcagcagtcacaggccaggcgtccacgggtcctgtgactcagatcatccagaccaaagggcccctgccagcgggaacaatcctgaagctggtgacctcagcagatggcaagcccaccaccatcatcactaccacgcaggccagtggggcggggaccaagcccaccatcctgggcatcagcagcgtctcccccagtaccaccaagcccggcacgaccaccatcatcaaaaccatccccatgtcggccatcatcacccaggcgggcgccacgggtgtgaccagcagtcctggcatcaagtcacccatcaccatcatcaccaccaaggtgatgacttcaggaactggagcacctgcgaaaatcatcactgctgtccccaaaattgccactggccacgggcagcagggagtgacccaggtggtgcttaagggggccccgggacagccaggcaccatcctccgcactgtgcccatggggggtgttcgcctggtcacacccgtcaccgtctccgccgtcaagccagccgtcaccacgttggttgtgaaaggcaccacaggtgtcacgaccctaggcacagtgacaggcaccgtctccaccagccttgccggggcggggggccacagcactagtgcttccctggccacgcccatcaccaccttgggcaccattgccaccctctcaagccaggtgatcaaccccactgccatcactgtgtcggccgcacagaccacgctgacagcggcaggcgggctcacaaccccgaccatcaccatgcagcccgtgtcccagcccacccaggtaactctgatcacggcacctagtggggtggaggcccagcctgtgcatgacctccctgtgtccattctggcctccccgactacagaacagcccaccgccacagttaccatcgccgactcaggccagggtgatgtgcagcctggcactgtcaccttggtgtgctccaacccaccctgtgagacccacgagactggcaccaccaacacggccaccactactgttgtggctaaccttgggggacacccccagcccacccaagtgcagttcgtctgtgacagacaggaggcagctgcttctcttgtgacctcgactgtgggccagcagaatggtagcgtggtccgagtctgttcgaacccgccctgcgagacccacgagacgggcaccaccaacaccgccaccaccgccacctccaacatggccgggcagcatggctgctcaaacccaccctgcgagacccacgagacgggcaccaccaacactgccactacagccatgtcgagcgtcggcgccaaccaccagcgagatgcccgtcgggcctgtgcagctggcacccctgccgtgatccggatcagtgtggccactggggcgctggaggcagcccagggctctaagccccagtgccaaacccgccagaccagcgcgaccagcaccaccatgactgtgatggccaccggggccccgtgctcggccggcccactccttgggccgagcatggcacgggagcccgggggccgcagccctgcttttgtgcagttggcccctctgagcagcaaagtcaggctgagcagcccaagcattaaggaccttcctgcggggcgccacagccatgcggtcagcaccgctgccatgacccgttccagcgtgggtgctggggagccccgcatggcacctgtgtgcgagagcctccagggtggctcgcccagcaccacagtgactgtgacagccctggaggcactgctgtgcccctcggccaccgtgacccaagtctgctccaacccaccatgtgagacccacgagacaggcaccaccaacaccgccactacctcgaatgcaggcagcgcccagagggtgtgctccaacccgccatgcgagacccacgagacgggcaccacccacacggccaccaccgctacttcaaacgggggcacgggccagcccgagggtgggcagcagccccctgctggtcgcccctgtgagacacaccagaccacttccactggcaccaccatgtcggtcagcgtgggtgccctgcttcccgacgccacttcttcccacaggaccgtggagtctggcctagaggtggcggcggcacccagcgtcaccccccaggctggcaccgcgctgctggctcctttcccaacacagagggtgtgctccaaccccccctgtgagacccacgagacgggcaccactcacacggccaccactgtcacttccaacatgagttcaaaccaagaccccccacctgctgccagcgatcagggagaggtggagagcacccagggcgacagcgtgaacatcaccagctccagtgccatcacgacaaccgtgtcctccacactgacgcgggctgtgaccaccgtgacgcagtccacaccggtcccgggcccctctgtgccgcccccagaggaactccaggtgtcgccaggtcctcgccagcagctgccaccacggcagcttctgcagtcggcttccacagccctgatgggggagtccgccgaggtcctgtcagcctcccagacccctgagctcccggccgccgtggatctgagcagcacaggggagccatcttcgggccaggagtctgccggctctgcggtggtggccactgtggtggtccagccacccccacccacacagtccgaagtagaccagttatcacttccccaagagctaatggccgaggcccaagctggcaccaccaccctcatggtaacggggctcacccccgaggagctggcagtgacggctgctgcagaagcagctgcccaggccgcagccacggaggaagcccaggccctggccatccaggcggtgctccaggccgcgcagcaggccgtcatgggcaccggcgagcccatggacacctccgaggcagcagcaaccgtgactcaggcggagctggggcacctgtcggccgagggtcaggagggccaggccaccaccatacccattgtgctgacacagcaggagctggctgccctggtgcagcagcagcagctgcaggaggcccaggcccagcagcagcatcaccacctccccactgaggccctggcccctgccgacagtctcaacgacccagccattgagagcaattgcctcaatgagctggccggcacggtccccagcactgtggcgctgctgccctcaacggccactgagagcctggctccatccaacacatttgtggccccccagccggttgtggtggccagcccagccaagctgcaggctgcagctaccctgaccgaagtggccaatggcatcgagtccctgggtgtgaagccagacctgccgcccccacccagcaaagcccccatgaagaaggaaaaccagtggtttgatgtgggagtcattaagggcaccaatgtaatggtgacacactatttcctgccaccagatgatgctgtcccatcagacgatgatttgggcaccgtccctgactataaccagctgaagaagcaggagctgcagccaggcacagcctataagtttcgtgttgccggaatcaatgcctgtgcgcgggggcccttcagcgaaatctcagcctttaagacgtgcctgcctggtttcccaggggccccttgtgccattaaaatcagcaaaagtccggatggtgctcacctcacctgggagccaccctctgtgacctccggcaagattatcgagtactccgtgtacctggccatccagagctcacaggctgggggcgagctcaagagctccaccccggcccagctggccttcatgcgggtgtactgcgggcccagcccctcctgcctggtgcagtcctccagcctttccaacgcccacatcgactacaccaccaagcccgccatcatcttccgcatcgccgcccgcaatgagaagggctatggcccggccacacaagtgaggtggctgcaggaaaccagtaaagacagctctggcaccaagccagccaacaagcggcccatgtcctctccagaaatgaaatctgctccaaagaaatctaaggccgatggtcagtgagaggaagctgactagcccctggattcttctccagacccccctgcttcaggaacacccgccagggcccacccctcccaccccgtcccagcattcgcacttcaccctcgcgagccgctgttcactcctctcccctttctctttctctctgtttttaaaataatctaaagaaagcacattttaccattgctgttgggaggaagcagaggcagatgggaaagcagagagaggagcgcgcttcctttcctccccgctgccgcccaccctggggagagacttttgcggggagggaaggcggacgtgaggacagccagctccccctcccaaggctgtgcgttcctgagggccaggtcgggggcaggcatggaggggaggaaaggcgtccctcttggccctccccagagtggctttcctggcaccctggcctgggtgtctggttctgttttcttttcttccccttgtgtttccagtcacctaacttcccttcctcaggctcccccggcccaccctgctcagtgaccccacaggaagcttacacattttctcagaggcctttgtgctcccacctcttctaccctccccctcttctttcccattttaaaaaagaaaagaaggaaaaagaaaaaaggggcaaggagccccgcggcggcctgggcagcgcctgtgcagacctccctgcaggccgcactgccaactgctgcatttgttgtgttttttaggttgcaattggtgaagttcacactttcattgtaattttagcgtgtggggttttgtcccttttttgttgttgttagctgtgtacagaatgtgtaatcttttttcttttctcttttttttgttttgttttgttttgtttttgtttttttacttttttcttcttggctaattcttggcagggatctttctggaggaaaagctggggccagccagggcaggagaggtgtgaaatctgccacgaggggcctgctgtttgccacccagcccaacttcctgttgctggcccctgccctctgcccttttgcctgtcctcaggccgctggaacaaaggaaggacagctcattcctcatgggcgatcactccgcatctatagggtcgagcctaggggagcttgagggagggctggggcctccttgtcctggatttccagctctccccatgccccctccctgagcaccaccggcaccgcctcccaaacagggctgctggtttccgcagccactgctccacctcccccaaatcgtcatggaaagggtggagaatggaggggaaccaggcgtccttggaggcagcttgggagggtgactgtgtagtgtcacccacaagggaggctagggcaatggagcaggccaccagcagcagctgtgcagcatggaactcaggccaggctccgaggctgggggatctgcttggagttttctgccccccaccccaaacttctgtcgaggagcaaggcttgccagcaagtcagaaggatttgaaccgagcagccaatctttccagccctcccctaccgacctctgcctggagacgcagcagcctgtgtcctccagggcctctggtttgttgtattatagtatatttcgctgtggaaaatgtcacgtttagtcaccttggagcccactcacctggtcctgttgttttaccccatcccttctctcgcgcgcctattgatttgtttctgaggagagtacaccgttcactattgtagagtaacccctgtgactcaatattaccatagtgcgatgtcgttttgtgctattttgaacaattaaaagactttttttgaaataa

SEQ ID NO: 37 - DNA, 4629 bp, Homo sapiens
  gene (1)...(4629): NFX1; nuclear transcription factor, X-box binding 1
acgtgacctggtgacagtgctgacttggctgtacagctcgatctaggttctgcggcacgggatggcggaggcgcctcctgtctcaggtacttttaaattcaatacagatgctgctgaattcattcctcaggagaaaaaaaattctggtctaaattgtgggactcaaaggagactagactctaataggattggtagaagaaattacagttcaccacctccctgtcacctttccaggcaggtcccttatgatgaaatctctgctgttcatcagcatagttatcatccgtcaggaagcaaacctaagagtcagcagacgtctttccagtcctctccttgtaataaatcgcccaagagccatggccttcagaatcaaccttggcagaaattgaggaatgagaagcaccatatcagagtcaagaaagcacagagtcttgctgagcagacctcagatacagctggattagagagctcgaccagatcagagagtgggacagacctcagagagcatagtccttctgagagtgagaaggaagttgtgggtgcagatcccaggggagcaaaacccaaaaaagcaacacagtttgtatacagctatggtagaggaccaaaagtcaaggggaaactcaaatgtgaatggagtaaccgaacaactccaaaaccggaggatgctggacccgaaagtaccaaacctgtgggggttttccaccctgactcttcagaggcatcctctagaaaaggagtattggatgggtatggagccagacgaaatgagcagagaagatacccacagaaaaggcctccctgggaagtggagggggccaggccacgaccaggcagaaatccaccaaaacaggagggccaccgacatacaaacgcaggacacagaaacaacatgggccccattccaaaggatgacctcaatgaaagaccagcaaaatctacctgtgacagtgagaacttggcagtcatcaacaagtcttccaggagggttgaccaagagaaatgcactgtacggaggcaggatcctcaagtagtatctcctttctcccgaggcaaacagaaccatgtgctaaagaatgtggaaacgcacacaggttctctaattgaacaactaacaacagaaaaatacgagtgcatggtgtgctgtgaattggttcgtgtcacggccccagtgtggagttgtcagagctgttaccatgtgtttcatttgaactgcataaagaaatgggcaaggtctccagcatctcaagcagatggccagagtggttggaggtgccctgcctgtcagaatgtttctgcacatgttcctaatacctacacttgtttctgtggcaaggtaaagaatcctgagtggagcagaaatgaaattccacatagctgtggtgaggtttgtagaaagaaacagcctggccaggactgcccacattcctgtaaccttctctgccatccaggaccctgcccaccctgccctgcctttatgacaaaaacatgtgaatgtggacgaaccaggcacacagttcgctgtggtcaggctgtctcagtccactgttctaacccatgtgagaatattttgaactgtggtcagcaccagtgtgctgagctgtgccatgggggtcagtgccagccttgccagatcattttgaaccaggtatgctattgcggcagcacctcccgagatgtgttatgtggaaccgatgtaggaaagtctgatggatttggggatttcagctgtttaaagatatgtggcaaggacttgaaatgcggtaaccatacatgttcgcaagtgtgccaccctcagccctgccagcaatgcccacggctcccccagctggtgcgctgttgcccctgtggccaaactcctctcagccaattgctagaacttggaagtagtagtcggaaaacatgcatggaccctgtgccttcatgtggaaaagtgtgcggcaagcctctgccttgtggttccttagatttcattcatacctgtgaaaagctctgccatgaaggagactgtggaccatgctctcgcacatcagttatttcctgcagatgctctttcagaacaaaggagcttccatgtaccagtctcaaaagtgaagatgctacatttatgtgtgacaagcggtgtaacaagaaacggttgtgtggacggcataaatgtaatgagatatgctgtgtggataaggagcacaagtgtcctttgatttgtgggaggaaactccgttgtggccttcataggtgtgaagaaccttgtcatcgtggaaactgccagacatgctggcaagccagttttgatgaattaacctgccattgtggtgcatcagtgatttaccctccagttccctgtggtactaggccccctgaatgtacccaaacctgcgctagagtccatgagtgtgaccatccagtatatcattcttgtcatagtgaggagaagtgtcccccttgcactttcctaactcagaagtggtgcatgggcaagcatgagtttcggagcaacatcccctgtcacctggttgatatctcttgcggattaccctgcagtgccacgctaccatgtgggatgcacaaatgtcagagactctgtcacaaaggggagtgtcttgtggatgagccctgcaagcagccctgcaccacccccagagctgactgtggtcacccgtgtatggcaccctgccataccagctcaccctgccctgtgactgcttgtaaagctaaggtagagctacagtgtgaatgtggacgaagaaaagagatggtgatttgctctgaagcatctagtacttatcaaagaatagctgcaatctccatggcctctaagataacagacatgcagcttggaggttcagtggagatcagcaagttaattaccaaaaaggaagttcatcaagccaggctggagtgtgatgaggagtgttcagccttggaaaggaaaaagagattagcagaggcatttcatatcagtgaggattctgatcctttcaatatacgttcttcagggtcaaaattcagtgatagtttgaaagaagatgccaggaaggacttaaagtttgtcagtgacgttgagaaggaaatggaaaccctcgtggaggccgtgaataagggaaagaatagtaagaaaagccacagcttccctcccatgaacagagaccaccgccggatcatccatgacttggcccaagtttatggcctggagagcgtgagctatgacagtgaaccgaagcgcaatgtggtggtcactgccatcagggggaagtccgtttgtcctcctaccacgctgacaggtgtgcttgaaagggaaatgcaggcacggcctccaccaccgattcctcatcacagacatcagtcagacaagaatcctgggagcagtaatttacagaaaataaccaaggagccaataattgactattttgacgtccaggactaagaagatcatgatgcacttagataaaagaatgattaggtatagtggagacttatttgccagcagataaatcatgcccgttcccctctgcctggcagaatcacagtctcacatactgtcttgtactgacacatccaaagcatgagtgtgtcagaaatcccttgtctattcctgtctgtataaagtgtttcattatgaccagatctctgattgtatggtcactaggtatgcaatcacgcattcaaagaggctctttacaccatcactgtgattgctctgagagttgagggactattgggctttatttggacaaaccaaacttttagcctgaaaccaactttatgccactaagtcatagcctcagttgtcccagttatttgtcctcctgaaaatgcctgaaacatcagacagacattgcttgctttacccaaactgatcaaaatctttaggagcacaaatgaattttttagtctgaaataccaaataatgaattggtataccatatccggaatcacacatgttatcttaaacccagccatcatacctaagtcttttgccaaaacctctcataggtatatctagctgaacttattttggcattttcaatgtgatcagttctagacctagaagggggtcaggctgctttacagaattctatttccttaagtccctggcacttctcataccacatcactgaacctgttcagtaacaatcagtttggccgtcccccatgatggtaggaaatatagagagcaagttcttctgccagggtcacactgtggtctctgaactgaccagtatatccctaactcctctttgatagagaaagagtctcaaatggacaactgtcctgtgttgctttccctaggccttcagcagcctattggctctccctgcctctgagctctggactctgtttgaatattccaagtagtatatggacagtccagggcttatgcccagcagcccactggaggcattcttcaggctcctttaaggcaggtgcattgatagttccattagtgtgacccttgcattggcacccctccagcctggaggccaggcttccagcaacttccttctgccctagagcaagccatgagccccagagcagtagcaggagacttgagaagtagagtgacaaaaacaagcacttaattaaattataaaatttaactttaaaaaaaaaaaaaaaaaaaaaaaaaaa

SEQ ID NO: 38 - DNA, 7984 bp, Homo sapiens
  gene (1)...(7984): CRSP2; cofactor required for Sp1 transcriptional activation, subunit 2a
cggcccggcggtaggcggtggcgactctgcccgctcccgtttcggcgcggtgaccgagcgcccgggaggctcgaggaccgcatcgtgtgccgttgcgccaagcccggtcctgcgccgccatggccccagtgcagctggagaaccaccagctggtcccgcccggaggcggcggcgggggcagcggcggacccccgtcagccccagcccctcctcccccgggagccgccgtggcggcggccgctgcagctgcggctagcccgggctaccggctgagcaccctcattgaatttctgctgcaccgggcctactcggagcttatggtgttgacggacctactgccaaggaaatctgatgtggaaaggaaaatagaaatagtgcagtttgctagccggacacgccaactcttcgttcgattattagctttagtgaaatgggctaataatgctggcaaagtggaaaaatgtgcgatgatttcaagctttttagatcagcaagccatcctgtttgtggacactgctgatcgcctggcctcgttagctagagatgctctggtccatgcacgcctgcctagttttgccatcccatatgccattgatgtactaactactggatcttacccacggctgccaacctgcattagggataaaattattcctccagacccaattaccaaaattgaaaaacaagccacactccatcagctgaatcagattcttagacatcggcttgtaaccacagatcttcctcctcagttagcaaatcttacagttgcaaatggccgggtgaagtttcgtgttgaaggagaatttgaagccaccttgactgtgatgggagatgaccctgatgttccatggcgtcttctcaagctagaaattctagttgaggataaggaaacaggagatgggcgagctttggttcatagcatgcaaatcagcttcatccatcaactggtgcagtctaggctctttgctgatgagaaacctcttcaggatatgtacaactgcctacattctttctgtttatcacttcagttagaagtgttacattcccaaactctaatgttaatccgagaacggtggggagaccttgtgcaggtggaaaggtatcatgctggaaagtgcctctccctttcagtttggaatcaacaggttcttgggagaaaaactggaacagcatctgttcacaaagttacaattaaaattgatgagaatgatgtctccaagcctttacagatttttcacgatcctcctttgccagcttctgattccaaattagtagaaagagccatgaagatcgaccacttatcaatagaaaaactcctgattgacagtgtccatgcaagagctcatcagaagctccaagaactgaaggccattcttagaggcttcaatgccaatgaaaactcttccatagagactgcactcccagctcttgttgttcccatcttggagccctgtggtaattcagagtgtctgcacatttttgtagatttacattctggaatgtttcaattgatgctttatggacttgaccaggccactctggatgacatggagaagtctgtgaatgatgatatgaaacgaatcataccctggattcagcaacttaagttttggcttggacaacagcgttgcaagcagtctataaaacatctgcctacgataagcagtgaaacattgcagctttccaattactcaactcatcctattggaaacctttctaagaataaactgttcattaaacttacccgccttccacaatactacattgttgtggagatgttggaggttcccaataaacccacacaactgtcgtacaagtactactttatgtctgtgaatgctgcagatcgtgaagacagccctgcaatggcattgctgctgcagcagttcaaggaaaacattcaggacttggtttttcgtacaaaaaccgggaaacagaccagaaccaatgccaagcgcaagttgtctgatgatccatgtccagtagaatccaagaaaacaaaacgagcaggagaaatgtgtgccttcaataaagttttagcccacttcgtcgctatgtgtgatacaaatatgccatttgtaggacttcggttggagttgtccaatctggagattccacatcaaggagtgcaagtggaaggtgatggcttcagccatgcaattcgcttattaaaaattcctccctgtaagggtataactgaggaaacccaaaaggctctggaccgctctcttcttgattgcactttccgattacaaggtagaaataaccgcacttgggtagcagagttagtgtttgcaaattgtccacttaatggcacttctactagggagcaaggaccatcccggcacgtttacctgacatatgaaaatctgttgtctgagcctgttggtggtagaaaggtggttgaaatgtttcttaatgactggaatagcattgcacgattatatgagtgtgtgttggaatttgcacgttctctaccagacatacctgctcatctaaatattttctcagaagttcgtgtttataattaccgaaaacttatcttgtgttatggaaccaccaagggaagctcaattagtatccaatggaattcgatccatcaaaaattccacatttctttgggaactgttggcccaaactcaggttgcagtaactgtcacaataccattctccatcagcttcaagaaatgttcaacaaaacaccaaatgtggttcagttattacaggtactgtttgatactcaggctccattaaatgccatcaacaaactccccactgtgccgatgttgggcttgacccagagaaccaatactgcctaccagtgcttctccattctgccacagtcgtccacccacatcagactggccttcaggaacatgtattgcattgatatatactgccggagtcgaggtgttgtggcaatacgggatggtgcctatagtctttttgataacagcaaactagttgaaggtttctatcctgcaccaggactaaagacgttcctgaatatgtttgttgacagcaatcaggatgctcgaagaaggtctgtaaatgaggacgataatcccccttctcctataggaggagatatgatggattctttaatatcgcagctccagccaccaccccagcaacagccatttccaaagcagccaggaacatcaggtgcttatcctcttacttcaccccctacatcttatcatagcacagtcaatcagtctccctcaatgatgcacacacagtctccaggaaatctgcatgctgccagctcccccagtggggctttgagagccccatcaccagcgtcatttgttccaactcctcccccatcctcgcatggaatctcaataggaccaggggccagttttgctagtccacatggaactcttgaccctagttccccatacactatggtgtcaccaagtggacgagcagggaactggccaggatctcctcaagtgtctggcccctcaccagcagcacgcatgcctggaatgtcaccagccaacccctcactacattctccggttccagatgcttctcattcccctcgagctggaacaagttctcaaacaatgccaacaaacatgcctccacctcgtaaactacctcagcgctcttgggcggcatccatacctaccatcctcactcacagtgccttgaacattttactgctgccctctccaactccaggccttgtgcccggcctggcaggtagttacctttgttctccacttgagagattccttggatcagtcatcatgagacgacatcttcaaagaattattcaacaagaaacgctgcagctgataaattctaatgaacccggagtgatcatgtttaagactgatgcactgaaatgcagagtagctcttagtcccaaaaccaaccaaacgcttcagctaaaagtgacacctgaaaatgcaggacagtggaaacctgatgaacttcaagttttggagaaattctttgaaacaagagttgcaggaccaccatttaaagctaatacgttaatagccttcaccaagctattaggagctcctacacacatcctcagggattgtgtgcacattatgaagctggagctgttccctgaccaagcaacacagctaaaatggaatgttcagttttgcctgacgatccctcccagcgcgccgccgattgcacctcctgggacgcctgctgtggtgctgaaatccaaaatgctattttttcttcaactaactcagaaaacatcggtccctccccaagaacctgttagtattatagttccaatcatttatgacatggcttcaggtacaacccagcaggcagacattcccagacagcagaactcttctgttgctgctcccatgatggtcagcaacattctgaagaggtttgcagagatgaatccaccacgacaaggtgaatgcacaatatttgcagctgttcgtgatttaatggctaatcttacactgccccctggtgggcgtccatagacactattgtttttaaaccaggaaggctgacagatgagacaacaaaaaaaatctgaattcagccttcagtttaaaaaaggaaaaggactttttttttaaactttaagagctaaatattctaaactggcaaaaattttcagggtgcacttctttcatcaaaatgatcatccatcttttgtataatgaacttgtatagtgtgtttaaatgggacacatttcaaaggaaataaatcagtgtccgtttgccagtaataaatttaatattctgttttatactataaagttatgaaaatgccaaacttgtttatttaattgttttcttatgttttgggtgtaatagtccttttttggtttttgttttgttttttaaggcaggtctgtcatttttgaatactgtaaaactgtgataaactttatattgaagctgtattttaatatgaccgctttgaatccttacatgaaatgttctgggagttgttataaacacaccccaaggaagcaatatttaataaaactaggtcaaaaacaaaacaaaacagtgacactcacttgtgtgtatataaactctagagacatcttaggccctcccttcatctttaacctggtggatggagccagttattaatttataataatacatgtctgaaatttgaccaaaaacatcttctttatgtcaaggttaatatattttcttcagacattcctaacttgactagtggttaacatttagcaccttggttgtctttcaatatataggatttgggtgaaaaataaggaacttagtctttgagaagggacagtatgtggttcgtattgatcaaggatcccaaataatgcaatcattctcatctgttgaggatgtgggaacttttctgtcaggaaaatttagtttacagaatacttctgcatacaacattaattaggaaacaaagaactttaatgctgtacctctgcatgaagtttctgtgagctttctttatcttgcttaacttatcctcctttccttccccaccaaaattgggattgttctaaacaatttcaatttaattcattgtcaacaaaacgatagaatgttttgcttggaagaaacctggtccattcccctcattgtgaagaatcccgaatcctgggcctgtccagggtcaaatggagtttttggtattaatacatctctgcagggtttctttttggagcactgtgctgtctttcctagtcagtcagggacttttttaagcttcaaagaacatggtctgatgcaaaggtcttctaccgacttcatggtatttaggaaaaatttttaagtgataataccatgccgagttacctggtcactgtaatactggttctccattaaaatgtagatgctttgtctaattccacttcaagatgatttaattgagcttctataactatcaactatcaaattctagtttcaagtatctcactgtacctatatcatatctgttctcaactttgtttgattctccactcattttcaatcagagttgaagccttctttgcctttcttctttgtcttatttctgccttcatctcccacttctcacccaactctgccactgttcaggagcagttgactaaacttttatccttcctttaacttaatctttttatccttctgtttttagttcttacagtggtaatcacatcagtttggcaatctaagatgtctttatcaagggtcagcccttttctatttggagctgaatcttttctttcatgtagaaatctacatgtatatacaagaaatgtatttttatctgcaggataatattgtttcaaataattttattaagagttaactttttaggtattttcccttttaatgtggttttgaaatgttaattgcatttttacacatgatgcattcagtggacactcttttttcctaattataactcatctctgaatgcattgtgccaatcaagagtaagataggattcagtgcgcaaaatttgcttttgatgcttattaagagtaaacccatgaatttgaatttttatattatgcttggtacttggaattgatatataccttattaaagcttgaaaacatttagcatttgaatagtaagtttcatcacaaatcatgtcttttgcctcttttgttacccaggttcgatcaaggggactggatgcccaagtgattttctctaataatcgggttgggacttaactttaaggtgttttcaatgtgattttagtatttacagtaataaataaaatttatacaaagtttcagagaactaccaactctctagttaattttgatgaagatgttttcattaatagtctggtttagtattaatttaaccttgaaataagaaattggtcctgatttgccaccttcatttttccatctgctgaaatgccatttcagaatgtataaaagtactttcagaattagaagaaaagtgagagcttcagcagaacgtgtcaagtcgctaataacaggagtcgaaccttatgtttgaaaattaactgagtgagttaagtgcaagcataaactaaaatttatatgactttgaatttaagaaccactaataactgtgttctaattaacacagtaatctgttcagccaaaggatactaacagccattctttggactgtggttaatttgataacttcaagaagttataactttgctaccctggattctgatgcaatttcaatataaaaagttgttcctttcatagtttttccccccaaaagaagtgcctattacattttaccttaatgtttatttatcatccaagtgttcttgatataaaatcagtatacagaaaattttaagagaacttacattcaagggacatagagggaaaatgttaatgtgtatttcattgtttcttgtggaatttttttttcattcggtttgagaatggacagtgtgtctaaggatgtgatttaaatttacttaggatggatttagcattgaggttattaagacaaagttagcagaggccagccagcactgtgctgcacaactccaggaactccatggtatgctcacaattttctgcaagttgcctccacttccaagtggccagtgatttgttaaaataccttgcccaagaatataactgattgatgaaagggcatatttaggtcaatataaaataatagcaagtatttactgagagcttactgtgtgctaggcattttcttaatatttcaatgtgtatttaatcctcacaacaacccaatgtggtaggttattgatatcctcttcttaaaaatgagtaaatggaggcccagagaaatgtaatttgcctgagatcaaattaaggttgcttaagatcaaactgctaagaaggggcagtttgagtctgagctagtgctcctaaacactggacatttttgctgtgaaccacagtcgggaaggtttaatgtttagactctttcctcagaggcttttaactacacacaattttgacatgttttccaagctcagaaagctcattaaaaacaactaaaactg

SEQ ID NO: 39 - DNA, 2986 bp, Homo sapiens
  gene (1)...(2986): ICAM1; intercellular adhesion molecule 1 (CD54), human rhinovirus receptor
gcgccccagtcgacgctgagctcctctgctactcagagttgcaacctcagcctcgctatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcgaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgaacctatcccgggacagggcctcttcctcggccttcccatattggtggcagtggtgccacactgaacagagtggaagacatatgccatgcagctacacctaccggccctgggacgccggaggacagggcattgtcctcagtcagatacaacagcatttggggccatggtacctgcacacctaaaacactaggccacgcatctgatctgtagtcacatgactaagccaagaggaaggagcaagactcaagacatgattgatggatgttaaagtctagcctgatgagaggggaagtggtgggggagacatagccccaccatgaggacatacaactgggaaatactgaaacttgctgcctattgggtatgctgaggcccacagacttacagaagaagtggccctccatagacatgtgtagcatcaaaacacaaaggcccacacttcctgacggatgccagcttgggcactgctgtctactgaccccaacccttgatgatatgtatttattcatttgttattttaccagctatttattgagtgtcttttatgtaggctaaatgaacataggtctctggcctcacggagctcccagtccatgtcacattcaaggtcaccaggtacagttgtacaggttgtacactgcaggagagtgcctggcaaaaagatcaaatggggctgggacttctcattggccaacctgcctttccccagaaggagtgatttttctatcggcacaaaagcactatatggactggtaatggttcacaggttcagagattacccagtgaggccttattcctcccttccccccaaaactgacacctttgttagccacctccccacccacatacatttctgccagtgttcacaatgacactcagcggtcatgtctggacatgagtgcccagggaatatgcccaagctatgccttgtcctcttgtcctgtttgcatttcactgggagcttgcactattgcagctccagtttcctgcagtgatcagggtcctgcaagcagtggggaagggggccaaggtattggaggactccctcccagctttggaagggtcatccgcgtgtgtgtgtgtgtgtatgtgtagacaagctctcgctctgtcacccaggctggagtgcagtggtgcaatcatggttcactgcagtcttgaccttttgggctcaagtgatcctcccacctcagcctcctgagtagctgggaccataggctcacaacaccacacctggcaaatttgattttttttttttttttcagagacggggtctcgcaacattgcccagacttcctttgtgttagttaataaagctttctcaactgcc

SEQ ID NO: 40 - DNA, 2004 bp, Homo sapiens
  gene (1)...(2004): PSG3; pregnancy specific beta-1-glycoprotein 3
gggcgggacaactggtctgagtactatggctgattttcgctgtctggcattgagaagccacacgccccttttgcttaggaggcctctctgctggaggatgacgatggcatggtttatctaaggccactgacaagtcatcaatataggacagcacagctgagagccatgctcaggaagtttctggatcctaggctcagctccacagaggagaacacgcaggcagcagagaccatggggcccctctcagcccctccctgcacacagcgcatcacctggaaggggctcctgctcacagcatcacttttaaacttctggaacccgcctaccactgcccaagtcacgattgaagccgagccaaccaaagtttccaaggggaaggacgttcttctacttgtccacaatttgccccagaatcttgctggctacatctggtacaaagggcaaatgaaggacctctaccattacattacatcatacgtagtagatggtcaaataattatatatgggcctgcatacagtggacgagaaacagtatattccaatgcatccctgctgatccagaatgtcacccgggaggacgcaggatcctacaccttacacatcgtaaagcgaggtgatgggactagaggagaaactggacatttcaccttcaccttatacctggagactcccaagccctccatctccagcagcaacttataccccagggaggacatggaggctgtgagcttaacctgtgatcctgagactccggacgcaagctacctgtggtggatgaatggtcagagcctccctatgactcacagcttgcagttgtccaaaaacaaaaggaccctctttctatttggtgtcacaaagtacactgcaggaccctatgaatgtgaaatacggaacccagtgagtgccagccgcagtgacccagtcaccctgaatctcctcccgaagctgcccaagccctacatcaccatcaacaacttaaaccccagggagaataaggatgtcttagccttcacctgtgaacctaagagtgagaactacacctacatttggtggctaaatggtcagagcctcccggtcagtcccagggtaaagcgacccattgaaaacaggatcctcattctacccagtgtcacgagaaatgaaacaggaccctatcaatgtgaaatacaggaccgatatggtggcatccgcagttacccagtcaccctgaatgtcctctatggtccagacctccccagaatttacccttcattcacctattaccattcaggagaaaacctctacttgtcctgcttcgcggactctaacccaccagcagaatattcttggacaattaatgggaagtttcagctatcaggacaaaagctctttatcccccagattactacaaagcatagcgggctctatgcttgctctgttcgtaactcagccactggcatggaaagctccaaatccatgacagtcaaagtctctgctccttcaggaacaggacatcttcctggccttaatccattatagcagccgtgatgtcatttctgtatttcaggaagactggcagacagttgctttcattcttcctcaaagtatttaccatcagctacagtccaaaattgctttttgttcaaggagatttatgaaaagactctgacaaggactcttgaatacaagttcctgataacttcaagatcatacatggactaagaactttcaaaattttaatgaacaggctgatacttcatgaaattcaagacaaagaaaaaaacccaattttattggactaaatagtcaaaacaatgttttcataattttctatttgaaaatgtgctgattctttgaatgttttattctccagatttatgcactttttttcttcagcaattggtaaagtatacttttgtaaacaaaaattgaaacatttgcttttgctccctaagtgccccagaattgggaaactattcaggagtattcatatgtttatggtaataaagttatctgcacaaaccc

SEQ ID NO: 41 - DNA, 2380 bp, Homo sapiens
  gene (1)...(2380): STC2; stanniocalcin 2
gaggaggagggaaaaggcgagcaaaaaggaagagtgggaggaggaggggaagcggcgaaggaggaagaggaggaggaggaagaggggagcacaaaggatccaggtctcccgacgggaggttaataccaagaaccatgtgtgccgagcggctgggccagttcatgaccctggctttggtgttggccacctttgacccggcgcgggggaccgacgccaccaacccacccgagggtccccaagacaggagctcccagcagaaaggccgcctgtccctgcagaatacagcggagatccagcactgtttggtcaacgctggcgatgtggggtgtggcgtgtttgaatgtttcgagaacaactcttgtgagattcggggcttacatgggatttgcatgacttttctgcacaacgctggaaaatttgatgcccagggcaagtcattcatcaaagacgccttgaaatgtaaggcccacgctctgcggcacaggttcggctgcataagccggaagtgcccggccatcagggaaatggtgtcccagttgcagcgggaatgctacctcaagcacgacctgtgcgcggctgcccaggagaacacccgggtgatagtggagatgatccatttcaaggacttgctgctgcacgaaccctacgtggacctcgtgaacttgctgctgacctgtggggaggaggtgaaggaggccatcacccacagcgtgcaggttcagtgtgagcagaactggggaagcctgtgctccatcttgagcttctgcacctcggccatccagaagcctcccacggcgccccccgagcgccagccccaggtggacagaaccaagctctccagggcccaccacggggaagcaggacatcacctcccagagcccagcagtagggagactggccgaggtgccaagggtgagcgaggtagcaagagccacccaaacgcccatgcccgaggcagagtcgggggccttggggctcagggaccttccggaagcagcgagtgggaagacgaacagtctgagtattctgatatccggaggtgaaatgaaaggcctggccacgaaatctttcctccacgccgtccattttcttatctatggacattccaaaacatttaccattagagaggggggatgtcacacgcaggattctgtggggactgtggacttcatcgaggtgtgtgttcgcggaacggacaggtgagatggagacccctggggccgtggggtctcaggggtgcctggtgaattctgcacttacacgtactcaagggagcgcgcccgcgttatcctcgtacctttgtcttctttccatctgtggagtcagtgggtgtcggccgctctgttgtgggggaggtgaaccagggaggggcagggcaaggcagggcccccagagctgggccacacagtgggtgctgggcctcgccccgaagcttctggtgcagcagcctctggtgctgtctccgcggaagtcagggcggctggattccaggacaggagtgaatgtaaaaataaatatcgcttagaatgcaggagaagggtggagaggaggcaggggccgagggggtgcttggtgccaaactgaaattcagtttcttgtgtggggccttgcggttcagagctcttggcgagggtggagggaggagtgtcatttctatgtgtaatttctgagccattgtactgtctgggctgggggggacactgtccaagggagtggcccctatgagtttatattttaaccactgcttcaaatctcgatttcactttttttatttatccagttatatctacatatctgtcatctaaataaatggctttcaaacaaagcaactgggtcattaaaaccagctcaaagggggtttaaaaaaaaaaaaaccagcccatcctttgaggctgatttttcttttttttaagttctattttaaaagctatcaaacagcgacatagccatacatctgactgcctgacatggactcctgcccacttgggggaaaccttatacccagaggaaaatacacacctggggagtacatttgacaaatttcccttaggatttcgttatctcaccttgaccctcagccaagattggtaaagctgcgtcctggcgattccaggagacccagctggaaacctggcttctccatgtgaggggatgggaaaggaaagaagagaatgaagactacttagtaattcccatcaggaaatgctgaccttttacataaaatcaaggagactgctgaaaatctctaagggacaggattttccagatcctaattggaaatttagcaataaggagaggagtccaaggggacaaataaaggcagagagagagagagagagagggagaggaagaaaagagagagagaaaagagcctcgtgcc

SEQ ID NO: 42 - DNA, 5189 bp, Homo sapiens
  gene (1)...(5189): SEMA3C; sema domain, immunoglobulin domain (Ig), (semaphorin) 3C
ggactgcgaaaggagcagggttgcggagctagggctccagcctgcggccgcgcattcttgcgtctggccagccgcgagctctaagggtcggccccgcccggtccgcccccgcggctccctgccaggctctcgcgggcgcgctcggggtggggcctcgcggctggcggagatgcggccggggctgcgcggtggtgatgcgagcctgctgggcggcgcgccggggcagccggagccgcgcgccgcggcgctgtaatcggacaccaagagcgctcgcccccggcctccggccactttccattcactccgaggtgcttgattgagcgacgcggagaagagctccgggtgccgcggcactgcagcgctgagattcctttacaaagaaactcagaggaccgggaagaaagaatttcacctttgcgacgtgctagaaaataaggtcgtctgggaaaaggactggagacacaagcgcatccaaccccggtagcaaactgatgacttttccgtgctgatttctttcaacctcggtattttcccttggatattaacttgcatatctgaagaaatggcattccggacaatttgcgtgttggttggagtatttatttgttctatctgtgtgaaaggatcttcccagccccaagcaagagtttatttaacatttgatgaacttcgagaaaccaagacctctgaatacttcagcctttcccaccatcctttagactacaggattttattaatggatgaagatcaggaccggatatatgtgggaagcaaagatcacattctttccctgaatattaacaatataagtcaagaagctttgagtgttttctggccagcatctacaatcaaagttgaagaatgcaaaatggctggcaaagatcccacacacggctgtgggaactttgtccgtgtaattcagactttcaatcgcacacatttgtatgtctgtgggagtggcgctttcagtcctgtctgtacttacttgaacagagggaggagatcagaggaccaagttttcatgattgactccaagtgtgaatctggaaaaggacgctgctctttcaaccccaacgtgaacacggtgtctgttatgatcaatgaggagcttttctctggaatgtatatagatttcatggggacagatgctgctatttttcgaagtttaaccaagaggaatgcggtcagaactgatcaacataattccaaatggctaagtgaacctatgtttgtagatgcacatgtcatcccagatggtactgatccaaatgatgctaaggtgtacttcttcttcaaagaaaaactgactgacaataacaggagcacgaaacagattcattccatgattgctcgaatatgtcctaatgacactggtggactgcgtagccttgtcaacaagtggaccactttcttaaaggcgaggctggtgtgctcggtaacagatgaagacggcccagaaacacactttgatgaattagaggatgtgtttctgctggaaactgataacccgaggacaacactagtgtatggcatttttacaacatcaagctcagttttcaaaggatcagccgtgtgtgtgtatcatttatctgatatacagactgtgtttaatgggccttttgcccacaaagaagggcccaatcatcagctgatttcctatcagggcagaattccatatcctcgccctggaacttgtccaggaggagcatttacacccaatatgcgaaccaccaaggagttcccagatgatgttgtcacttttattcggaaccatcctctcatgtacaattccatctacccaatccacaaaaggcctttgattgttcgtattggcactgactacaagtatacaaagatagctgtggatcgagtgaacgctgctgatgggagataccatgtcctgtttctcggaacagatcggggtactgtgcaaaaagtggttgttcttcctactaacaactctgtcagtggcgagctcattctggaggagctggaagtctttaagaatcatgctcctataacaacaatgaaaatttcatctaaaaagcaacagttgtatgtgagttccaatgaaggggtttcccaggtatctctgcaccgctgccacatctatggtacagcctgtgctgactgctgcctggcgcgggacccttattgcgcctgggatggccattcctgttccagattctacccaactgggaaacggaggagccgaagacaagatgtgagacatggaaacccactgactcaatgcagaggatttaatctaaaagcatacagaaatgcagctgaaattgtgcagtatggagtaaaaaataacaccacttttctggagtgtgcccccaagtctccgcaggcatctatcaagtggctgttacagaaagacaaagacaggaggaaagaggttaagctgaatgaacgaataatagccacttcacagggactcctgatccgctctgttcagggttctgaccaaggactttatcactgcattgctacagaaaatagtttcaagcagaccatagccaagatcaacttcaaagttttagattcagaaatggtggctgttgtgacggacaaatggtccccatggacctgggccagctctgtgagggctttacccttccacccgaaggacatcatgggggcattcagccactcagaaatgcagatgattaaccaatattgcaaagacactcggcagcaacatcagcagggagatgaatcacagaaaatgagaggggactatggcaagttaaaggccctcatcaatagtcggaaaagtagaaacaggaggaatcagttgccagagtcataatattttcttatgtgggtcttatgcttccattaacaaatgctctgtcttcaatgatcaaattttgagcaaagaaacttgtgctttaccaaggggaattactgaaaaaggtgattactcctgaagtgagttttacacgaactgaaatgagcatgcattttcttgtatgatagtgactagcactagacatgtcatggtcctcatggtgcatataaatatatttaacttaacccagattttatttatatctttattcaccttttcttcaaaatcgatatggtggctgcaaaactagaattgttgcatccctcaattgaatgagggccatatccctgtggtattcctttcctgctttggggctttagaattctaattgtcagtgattttgtatatgaaaacaagttccaaatccacagcttttacgtagtaaaagtcataaatgcatatgacagaatggctatcaaaagaaatagaaaaggaagacggcatttaaagttgtataaaaacacgagttattcataaagagaaaatgatgagtttttatggttccaatgaaatatgttggggtttttttaagattgtaaaaataatcagttactggtatctgtcactgacctttgtttccttattcaggaagataaaaatcagtaacctaccccatgaagatatttggtgggagttatatcagtgaagcagtttggtttatattcttatgttatcaccttccaaacaaaagcacttactttttttggaagttatttattttagactcaaagaatataatcttgcactactcagttattactgtttgttctcttattccctagtctgtgtggcaaattaaacaatataagaaggaaaaatttgaagtattagacttctaaataaggggtgaaatcatcagaaagaaaaatcaaagtagaaactactaattttttaagaggaatttataacaaatatggctagttttcaacttcagtactcaaattcaatgattcttccttttattaaaaccagtctcagatatcatactgatttttaagtcaacactatatattttatgatcttttcagtgtgatggcaaggtgcttgttatgtctagaaagtaagaaaacaatatgaggagacattctgtctttcaaaaggtaatggtacatacgttcactggtctctaagtgtaaaagtagtaaattttgtgatgaataaaataattatctcctaattgtatgttagaataattttattagaataatttcatactgaaattattttctccaaataaaaattagatggaaaaatgtgaaaaaaattattcatgctctcatatatattttaaaaacactacttttgcttttttatttaccttttaagacattttcatgcttccaggtaaaaacagatattgtaccatgtacctaatccaaatatcatataaacattttatttatagttaataatctatgatgaaggtaattaaagtagattatggcctttttaagtattgcagtctaaaacttcaaaaactaaaatcattgtcaaaattaatatgattattaatcagaatatcagaatatgattcactatttaaactatgataaattatgataatatatgaggaggcctcgctatagcaaaaatagttaaaatgctgacataacaccaaacttcattttttaaaaaatctgttgttccaaatgtgtataattttaaagtaatttctaaagcagtttattataatggtttgcctgcttaaaaggtataattaaacttcttttctcttctacattgacacacagaaatgtgtcaatgtaaagccaaaaccatcttctgtgtttatggccaatctattctcaaagttaaaagtaaaattgtttcagagtcacagttccctttatttcacataagcccaaactgatagacagtaacggtgtttagttttatactatatttgtgctatttaattctttctattttcacaattattaaattgtgtacactttcattacttttaaaaatgtagaaattcttcatgaacataactctgctgaatgtaaaagaaaattttttttcaaaaatgctgttaatgtatactactggtggttgattggttttattttatgtagcttgacaattcagtgacttaatatctattccatttgtattgtacataaaattttctagaaatacacttttttccaaagtgtaagtttgtgaatagattttagcatgatgaaactgtcataatggtgaatgttcaatctgtgtaagaaaacaaactaaatgtagttgtcacactaaaatttaattggatattgatgaaatcattggcctggcaaaataaaacatgttgaattccccaaaaaaaaaaaaaaaa

SEQ ID NO: 43 - protein, 453 aa, Homo sapiens
  PROPEP (1)...(453): Fibrinogen Gamma Chain
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQVRPEHPAETEYDSLYPEDDL

SEQ ID NO: 44 - protein, 3176 aa, Homo sapiens
  PROPEP (1)...(3176): Mature Form of Collagen alpha 3(VI) chain
MRKHRHLPLVAVFCLFLSGFPTTHAQQQQADVKNGAAADIIFLVDSSWTIGEEHFQLVREFLYDVVKSLAVGENDFHFALVQFNGNPHTEFLLNTYRTKQEVLSHISNMSYIGGTNQTGKGLEYIMQSHLTKAAGSRAGDGVPQVIVVLTDGHSKDGLALPSAELKSADVNVFAIGVEDADEGALKEIASEPLNMHMFNLENFTSLHDIVGNLVSCVHSSVSPERAGDTETLKDITAQDSADIIFLIDGSNNTGSVNFAVILDFLVNLLEKLPIGTQQIRVGVVQFSDEPRTMFSLDTYSTKAQVLGAVKALGFAGGELANIGLALDFVVENHFTRAGGSRVEEGVPQVLVLISAGPSSDEIRYGVVALKQASVFSFGLGAQAASRAELQHIATDDNLVFTVPEFRSFGDLQEKLLPYIVGVAQRHIVLKPPTIVTQVIEVNKRDIVFLVDGSSALGLANFNAIRDFIAKVIQRLEIGQDLIQVAVAQYADTVRPEFYFNTHPTKREVITAVRKMKPLDGSALYTGSALDFVRNNLFTSSAGYRAAEGIPKLLVLITGGKSLDEISQPAQELKRSSIMAFAIGNKGADQAELEEIAFDSSLVFIPAEFRAAPLQGMLPGLLAPLRTLSGTPEVHSNKRDIIFLLDGSANVGKTNFPYVRDFVMNLVNSLDIGNDNIRVGLVQFSDTPVTEFSLNTYQTKSDILGHLRQLQLQGGSGLNTGSALSYVYANHFTEAGGSRIREHVPQLLLLLTAGQSEDSYLQAANALTRAGILTFCVGASQANKAELEQIAFNPSLVYLMDDFSSLPALPQQLIQPLTTYVSGGVEEVPLAQPESKRDILFLFDGSANLVGQFPVVRDFLYKIIDELNVKPEGTRIAVAQYSDDVKVESRFDEHQSKPEILNLVKRMKIKTGKALNLGYALDYAQRYIFVKSAGSRIEDGVLQFLVLLVAGRSSDRVDGPASNLKQSGVVPFIFQAKNADPAELEQIVLSPAFILAAESLPKIGDLHPQIVNLLKSVHNGAPAPVSGEKDVVFLLDGSEGVRSGFPLLKEFVQRVVESLDVGQDRVRVAVVQYSDRTRPEFYLNSYMNKQDVVNAVRQLTLLGGPTPNTGAALEFVLRNILVSSAGSRITEGVPQLLIVLTADRSGDDVRNPSVVVKRGGAVPIGIGIGNADITEMQTISFIPDFAVAIPTFRQLGTVQQVISERVTQLTREELSRLQPVLQPLPSPGVGGKRDVVFLIDGSQSAGPEFQYVRTLIERLVDYLDVGFDTTRVAVIQFSDDPKAEFLLNAHSSKDEVQNAVQRLRPKGGRQINVGNALEYVSRNIFKRPLGSRIEEGVPQFLVLISSGKSDDEVVVPAVELKQFGVAPFTIARNADQEELVKISLSPEYVFSVSTFRELPSLEQKLLTPITTLTSEQIQKLLASTRYPPPAVESDAADIVFLIDSSEGVRPDGFAHIRDFVSRIVRRLNIGPSKVRVGVVQFSNDVFPEFYLKTYRSQAPVLDAIRRLRLRGGSPLNTGKALEFVARNLFVKSAGSRIEDGVPQHLVLVLGGKSQDDVSRFAQVIRSSGIVSLGVGDRNIDRTELQTITNDPRLVFTVREFRELPNIEERIMNSFGPSAATPAPPGVDTPPPSRPEKKKADIVFLLDGSINFRRDSFQEVLRFVSEIVDTVYEDGDSIQVGLVQYNSDPTDEFFLKDFSTKRQIIDAINKVVYKGGRHANTKVGLEHLRVNHFVPEAGSRLDQRVPQIAFVITGGKSVEDAQDVSLALTQRGVKVFAVGVRNIDSEEVGKIASNSATAFRVGNVQELSELSEQVLETLHDAMHETLCPGVTDAAKACNLDVILGFDGSRDQNVFVAQKGFESKVDAILNRISQMHRVSCSGGRSPTVRVSVVANTPSGPVEAFDFDEYQPEMLEKFRNMRSQHPYVLTEDTLKVYLNKFRQSSPDSVKVVIHFTDGADGDLADLHRASENLRQEGVRALILVGLERVVNLERLMHLEFGRGFMYDRPLRLNLLDLDYELAEQLDNIAEKACCGVPCKCSGQRGDRGPIGSIGPKGIPGEDGYRGYPGDEGGPGERGPPGVNGTQGFQGCPGQRGVKGSRGFPGEKGEVGEIGLDGLDGEDGDKGLPGSSGEKGNPGRRGDKGPRGEKGERGDVGIRGDPGNPGQDSQERGPKGETGDLGPMGVPGRDGVPGGPGETGKNGGFGRRGPPGAKGNKGGPGQPGFEGEQGTRGAQGPAGPAGPPGLIGEQGISGPRGSGGARGAPGERGRTGPLGRKGEPGEPGPKGGIGNPGPRGETGDDGRDGVGSEGRRGKKGERGFPGYPGPKGNPGEPGLNGTTGPKGIRGRRGNSGPPGIVGQKGRPGYPGPAGPRGNRGDSIDQCALIQSIKDKCPCCYGPLECPVFPTELAFALDTSEGVNQDTFGRMRDVVLSIVNVLTIAESNCPTGARVAVVTYNNEVTTEIRFADSKRKSVLLDKIKNLQVALTSKQQSLETAMSFVARNTFKRVRNGFLMRKVAVFFSNTPTRASPQLREAVLKLSDAGITPLFLTRQEDRQLINALQINNTAVGHALVLPAGRDLTDFLENVLTCHVCLDICNIDPSCGFGSWRPSFRDRRAAGSDVDIDMAFILDSAETTTLFQFNEMKKYIAYLVRQLDMSPDPKASQHFARVAVVQHAPSESVDNASMPPVKVEFSLTDYGSKEKLVDFLSRGMTQLQGTRALGSAIEYTIENVFESAPNPRDLKIVVLMLTGEVPEQQLEEAQRVILQAKCKGYFFVVLGIGRKVNIKEVYTFASEPNDVFFKLVDKSTELNEEPLMRFGRLLPSFVSSENAFYLSPDIRKQCDWFQGDQPTKNLVKFGHKQVNVPNNVTSSPTSNPVTTTKPVTTTKPVTTTTKPVTTTTKPVTIINQPSVKPAAAKPAPAKPVAAKPVATKTATVRPPVAVKPATAAKPVAAKPAAVRPPAAAAKPVATKPEVPRPQAAKPAATKPATTKPVVKMLREVQVFEITENSAKLHWERPEPPGPYFYDLTVTSAHDQSLVLKQNLTVTDRVIGGLLAGQTYHVAVVCYLRSQVRATYHGSFSTKKSQPPPPQPARSASSSTINLMVSTEPLALTETDICKLPKDEGTCRDFILKWYYDPNTKSCARFWYGGCGGNENKFGSQKECEKVCAPVLAKPGVISVMGT

SEQ ID NO: 45 - protein, 688 aa, Homo sapiens
  PROPEP (1)...(688): Mature Form of Complement C1s (C1 esterase)
MWCIVLFSLLAWVYAEPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 46 - protein, 128 aa, Homo sapiens
  PROPEP (1)...(128): CD59
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 47 - protein, 605 aa, Homo sapiens
  PROPEP (1)...(605): Insulin like growth factor binding complex acid labile chain (ALS)
MALRKGGLALALLLLSWVALGPRSLEGADPGTPGEAEGPACPAACVCSYDDDADELSVFCSSRNLTRLPDGVPGGTQALWLDGNNLSSVPPAAFQNLSSLGFLNLQGGQLGSLEPQALLGLENLCHLHLERNQLRSLALGTFAHTPALASLGLSNNRLSRLEDGLFEGLGSLWDLNLGWNSLAVLPDAAFRGLGSLRELVLAGNRLAYLQPALFSGLAELRELDLSRNALRAIKANVFVQLPRLQKLYLDRNLIAAVAPGAFLGLKALRWLDLSHNRVAGLLEDTFPGLLGLRVLRLSHNAIASLRPRTFKDLHFLEELQLGHNRIRQLAERSFEGLGQLEVLTLDHNQLQEVKAGAFLGLTNVAVMNLSGNCLRNLPEQVFRGLGKLHSLHLEGSCLGRIRPHTFTGLSGLRRLFLKDNGLVGIEEQSLWGLAELLELDLTSNQLTHLPHRLFQGLGKLEYLLLSRNRLAELPADALGPLQRAFWLDVSHNRLEALPNSLLAPLGRLRYLSLRNNSLRTFTPQPPGLERLWLEGNPWDCGCPLKALRDFALQNPSAVPRFVQAICEGDDCQPPAYTYNNITCASPPEVVGLDLRDLSEAHFAPC

SEQ ID NO: 48 - protein, 94 aa, Homo sapiens
  PROPEP (1)...(94): Defensin 5
MRTIAILAAILLVALQAQAESLQERADEATTQKQSGEDNQDLAISFAGNGLSALRTSGSQARATCYCRTGRCATRESLSGVCEISGRLYRLCCR

SEQ ID NO: 49 - protein, 151 aa, Homo sapiens
  PROPEP (1)...(151): Proline Rich Acidic Protein
MRRLLLVTSLVVVLLWEAGAVPAPKVPIKMQVKHWPSEQDPEKAWGARVVEPPEKDDQLVVLFPVQKPKLLTTEEKPRGQGRGPILPGTKAWMETEDTLGRVLSPEPDHDSLYHPPPEEDQGEERPRLWVMPNHQVLLGPEEDQDHIYHPQ

SEQ ID NO: 50 - protein, 949 aa, Homo sapiens
  PROPEP (1)...(949): Emilin-3
MILSLLFSLGGPLGWGLLGAWAQASSTSLSDLQSSRTPGVWKAEAEDTSKDPVGRNWCPYPMSKLVTLLALCKTEKFLIHSQQPCPQGAPDCQKVKVMYRMAHKPVYQVKQKVLTSLAWRCCPGYTGPNCEHHDSMAIPEPADPGDSHQEPQDGPVSFKPGHLAAVINEVEVQQEQQEHLLGDLQNDVHRVADSLPGLWKALPGNLTAAVMEANQTGHEFPDRSLEQVLLPHVDTFLQVHFSPIWRSFNQSLHSLTQAIRNLSLDVEANRQAISRVQDSAVARADFQELGAKFEAKVQENTQRVGQLRQDVEDRLHAQHFTLHRSISELQADVDTKLKRLHKAQEAPGTNGSLVLATPGAGARPEPDSLQARLGQLQRNLSELHMTTARREEELQYTLEDMRATLTRHVDEIKELYSESDETFDQISKVERQVEELQVNHTALRELRVILMEKSLIMEENKEEVERQLLELNLTLQHLQGGHADLIKYVKDCNCQKLYLDLDVIREGQRDATRALEETQVSLDERRQLDGSSLQALQNAVDAVSLAVDAHKAEGERARAATSRLRSQVQALDDEVGALKAAAAEARHEVRQLHSAFAALLEDALRHEAVLAALFGEEVLEEMSEQTPGPLPLSYEQIRVALQDAASGLQEQALGWDELAARVTALEQASEPPRPAEHLEPSHDAGREEAATTALAGLARELQSLSNDVKNVGRCCEAEAGAGAASLNASLDGLHNALFATQRSLEQHQRLFHSLFGNFQGLMEANVSLDLGKLQTMLSRKGKKQQKDLEAPRKRDKKEAEPLVDIRVTGPVPGALGAALWEAGSPVAFYASFSEGTAALQTVKFNTTYINIGSSYFPEHGYFRAPERGVYLFAVSVEFGPGPGTGQLVFGGHHRTPVCTTGQGSGSTATVFAMAELQKGERVWFELTQGSITKRSLSGTAFGGFLMFKT

SEQ ID NO: 51 - protein, 185 aa, Homo sapiens
  PROPEP (1)...(185): CA11 Protein
MKFTIVFAGLLGVFLAPALANYNINVNDDNNNAGSGQQSVSVNNEHNVANVDNNNGWDSWNSIWDYGNGFAATRLFQKKTCIVHKMNKEVMPSIQSLDALVKEKKLQGKGPGGPPPKGLMYSVNPNKVDDLSKFGKNIANMCRGIPTYMAEEMQEASLFFYSGTCYTTSVLWIVDISFCGDTVEN

SEQ ID NO: 52 - protein, 1676 aa, Homo sapiens
  PROPEP (1)...(1676): C5A Anaphylotoxin
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVILNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGIGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 53 - protein, 161 aa, Homo sapiens
  PROPEP (1)...(161): Nonsecretory ribonuclease isoform (three moieties)
MVPKLFTSQICLLLLLGLLAVEGSLHVKPPQFTWAQWFETQHINMTSQQCTNAMQVINNYQRRCKNQNTFLLTTFANVVNVCGNPNMTCPSNKTRKNCHHSGSQVPLIHCNLTTPSPQNISNCRYAQTPANMFYIVACDNRDQRRDPPQYPVVPVHLDRII

SEQ ID NO: 54 - protein, 241 aa, Homo sapiens
  PROPEP (1)...(241): Glutathione transferase omega 1
MSGESARSLGKGSAPPGPVPEGSIRIYSMRFCPFAERTRLVLKAKGIRHEVININLKNKPEWFFKKNPFGLVPVLENSQGQLIYESAITCEYLDEAYPGKKLLPDDPYEKACQKMILELFSKVPSLVGSFIRSQNKEDYAGLKEEFRKEFTKLEEVLTNKKTTFFGGNSISMIDYLIWPWFERLEAMKLNECVDHTPKLKLWMAAMKEDPTVSALLTSEKDWQGFLELYLQNSPEACDYGL

SEQ ID NO: 55 - protein, 330 aa, Homo sapiens
  PROPEP (1)...(330): Complement factor H-related protein 1 (FHR-1)
MWLLVSVILISRISSVGGEATFCDFPKINHGILYDEEKYKPFSQVPTGEVFYYSCEYNFVSPSKSFWTRITCTEEGWSPTPKCLRLCFFPFVENGHSESSGQTHLEGDTVQIICNTGYRLQNNENNISCVERGWSTPPKCRSTDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYLRTGESAEFVCKRGYRLSSRSHTLRTTCWDGKLEYPTCAKR

SEQ ID NO: 56 - protein, 211 aa, Homo sapiens
  PROPEP (1)...(211): Secreted phosphoprotein 24 (SPP-24)
MISRMEKMTMMMKILIMFALGMNYWSCSGFPVYDYDPSSLRDALSASVVKVNSQSLSPYLFRAFRSSLKRVEVLDENNLVMNLEFSIRETTCRKDSGEDPATCAFQRDYYVSTAVCRSTVKVSAQQVQGVHARCSWSSSTSESYSSEEMIFGDMLGSHKWRNNYLFGLISDESISEQFYDRSLGIMRRVLPPGNRRYPNHRHRARINTDFE

SEQ ID NO: 57 - protein, 466 aa, Homo sapiens
  PEPTIDE (1)...(466): Mature form of chitotriosidase
MVRSVAWAGFMVLLMIPWGSAAKLVCYFTNWAQYRQGEARFLPKDLDPSLCTHLIYAFAGMTNHQLSTTEWNDETLYQEFNGLKKMNPKLKTLLAIGGWNFGTQKFTDMVATANNRQTFVNSAIRFLRKYSFDGLDLDWEYPGSQGSPAVDKERFTTLVQDLANAFQQEAQTSGKERLLLSAAVPAGQTYVDAGYEVDKIAQNLDFVNLMAYDFHGSWEKVTGHNSPLYKRQEESGAAASLNVDAAVQQWLQKGTPASKLILGMPTYGRSFTLASSSDTRVGAPATGSGTPGPFTKEGGMLAYYEVCSWKGATKQRIQDQKVPYIFRDNQWVGFDDVESFKTKVSYLKQKGLGGAMVWALDLDDFAGFSCNQGRYPLIQTLRQELSLPYLPSGTPELEVPKPGQPSEPEHGPSPGQDTFCQGKADGLYPNPRERSSFYSCAAGRLFQQSCPTGLVFSNSCKCCTWN

SEQ ID NO: 58 - DNA, 25 bp, Homo sapiens
  primer_bind (1)...(25): Primer 212788_x_at
tctggaaggcgtgagccacttcttc

SEQ ID NO: 59 - DNA, 25 bp, Homo sapiens
  primer_bind (1)...(25): Primer 212788_x_at
gctacgagcgtctcctgaagatgca

SEQ ID NO: 60 - DNA, 25 bp, Homo sapiens
  protein_bind (1)...(25): Primer 212788_x_at
aaaccccagacgccatgaaagctgc

SEQ ID NO: 61 - DNA, 25 bp, Homo sapiens
  primer_bind (1)...(25): Primer 212788_x_at
tgaaagctgccatggccctggagaa

SEQ ID NO: 62 - DNA, 25 bp, Homo sapiens
  primer_bind (1)...(25): Primer 212788_x_at
ctctgtgacttcctggagactcact

SEQ ID NO: 63 - DNA, 25 bp, Homo sapiens
  primer_bind (1)...(25): Primer 212788_x_at
ggctgggcgagtatctcttcgaaag

SEQ ID NO: 64 - DNA, 25 bp, Homo sapiens
  primer_bind (1)...(25): Primer 212788_x_at
tcgaaaggctcactctcaagcacga

SEQ ID NO: 65 - DNA, 25 bp, Homo sapiens
  primer_bind (1)...(25): Primer 212788_x_at
agcacgactaagagccttctgagcc

SEQ ID NO: 66 - DNA, 25 bp, Homo sapiens
  protein_bind (1)...(25): Primer 212788_x_at
gagcccagcgacttctgaagggccc

SEQ ID NO: 67 - DNA, 25 bp, Homo sapiens
  primer_bind (1)...(25): Primer 212788_x_at
tccctccagccaataggcagctttc

SEQ ID NO: 68 - DNA, 25 bp, Homo sapiens
  primer_bind (1)...(25): Primer 212788_x_at
gcagctttcttaactatcctaacaa

SEQ ID NO: 69 - DNA, 25 bp, Homo sapiens
  primer_bind (1)...(25): Primer 40850_at
agaccgccttgtaccggaaaatgct

SEQ ID NO: 70 - DNA, 25 bp, Homo sapiens
  primer_bind (1)...(25): Primer 40850_at
gcaagggtgcctggtccatcccatg

SEQ ID NO: 71 - DNA, 25 bp, Homo sapiens
  primer_bind (1)...(25): Primer 40850_at
gtgcctggtccatcccatggaagtg

SEQ ID NO: 72 - DNA, 25 bp, Homo sapiens
  primer_bind (1)...(25): Primer 40850_at
ccatcccatggaagtggctgtttgg

SEQ ID NO: 73 - DNA, 25 bp, Homo sapiens
  primer_bind (1)...(25): Primer 40850_at
tgtttggggcgactgctgttgcctt

SEQ ID NO: 74 - DNA, 25 bp, Homo sapiens
  primer_bind (1)...(25): Primer 40850_at
actgaggccctctaggaggaaagcc

SEQ ID NO: 75 - DNA, 25 bp, Homo sapiens
  primer_bind (1)...(25): Primer 40850_at
ctgaggccctctaggaggaaagccc

SEQ ID NO: 76 - DNA, 25 bp, Homo sapiens
  primer_bind (1)...(25): Primer 40850_at
gaggccctctaggaggaaagcccag

SEQ ID NO: 77 - DNA, 25 bp, Homo sapiens
  primer_bind (1)...(25): Primer 40850_at
aggccctctaggaggaaagcccaga

SEQ ID NO: 78 - DNA, 25 bp, Homo sapiens
  primer_bind (1)...(25): Primer 40850_at
ggccctctaggaggaaagcccagag

SEQ ID NO: 79 - DNA, 25 bp, Homo sapiens
  primer_bind (1)...(25): Primer 40850_at
gccctctaggaggaaagcccagagg

SEQ ID NO: 80 - DNA, 25 bp, Homo sapiens
  primer_bind (1)...(25): Primer 40850_at
cctctaggaggaaagcccagaggga

SEQ ID NO: 81 - DNA, 25 bp, Homo sapiens
  primer_bind (1)...(25): Primer 40850_at
taggtctccgccagggctggcctca

SEQ ID NO: 82 - DNA, 25 bp, Homo sapiens
  primer_bind (1)...(25): 40850_at
agggctggcctcagtttctcctcaa

SEQ ID NO: 83 - DNA, 25 bp, Homo sapiens
  primer_bind (1)...(25): Primer 40850_at
ggctggcctcagtttctcctcaaca

SEQ ID NO: 84 - DNA, 25 bp, Homo sapiens
  primer_bind (1)...(25): Primer 40850_at
agtttctcctcaacaggcctggggg

SEQ ID NO: 85 - DNA, 25 bp, Homo sapiens
  primer_bind (1)...(25): Primer 212639_x_at
gatcaccaatgcttgctttgagcca

SEQ ID NO: 86 - DNA, 25 bp, Homo sapiens
  primer_bind (1)...(25): Primer 212639_x_at
gctttgagccagccaaccagatggt

SEQ ID NO: 87 - DNA, 25 bp, Homo sapiens
  primer_bind (1)...(25): Primer 212639_x_at
aaatgtgaccctcgccatggtaaat

SEQ ID NO: 88 - DNA, 25 bp, Homo sapiens
  primer_bind (1)...(25): Primer 212639_x_at
ccgtggtgacgtggttcccaaagat

SEQ ID NO: 89 - DNA, 25 bp, Homo sapiens
  primer_bind (1)...(25): Primer 212639_x_at
atgtcaatgctgccattgccaccat

SEQ ID NO: 90 - DNA, 25 bp, Homo sapiens
  primer_bind (1)...(25): Primer 212639_x_at
agtttgtggattggtgccccactgg

SEQ ID NO: 91 - DNA, 25 bp, Homo sapiens
  primer_bind (1)...(25): Primer 212639_x_at
ctcccactgtggtgcctggtggaga

SEQ ID NO: 92 - DNA, 25 bp, Homo sapiens
  primer_bind (1)...(25): Primer 212639_x_at
gagagctgtgtgcatgctgagcaac

SEQ ID NO: 93 - DNA, 25 bp, Homo sapiens
  primer_bind (1)...(25): Primer 212639_x_at
gcctttgttcactggtacgtgggtg

SEQ ID NO: 94 - DNA, 25 bp, Homo sapiens
  primer_bind (1)...(25): Primer 212639_x_at
ggcccgtgaagatatggctgccctt

SEQ ID NO: 95 - DNA, 25 bp, Homo sapiens
  primer_bind (1)...(25): Primer 212639_x_at
ctaattatccattccttttggccct

SEQ ID NO: 96 - DNA, 25 bp, Homo sapiens
  primer_bind (1)...(25): Primer 39854_r_at
atgcgcaacaacctctcgctggggg

SEQ ID NO: 97 - DNA, 25 bp, Homo sapiens
  primer_bind (1)...(25): Primer 39854_r_at
ccgaagctctgcgcatgcgcgcacc

SEQ ID NO: 98 - DNA, 25 bp, Homo sapiens
  primer_bind (1)...(25): Primer 39854_r_at
ccccgcggacccagcatccccgcag

SEQ ID NO: 99 - DNA, 25 bp, Homo sapiens
  primer_bind (1)...(25): Primer 39854_r_at
ccgcggacccagcatccccgcagca

SEQ ID NO: 100 - DNA, 25 bp, Homo sapiens
  primer_bind (1)...(25): Primer 39854_r_at
cctgctcccgaggcccggcccgtga

SEQ ID NO: 101 - DNA, 25 bp, Homo sapiens
  primer_bind (1)...(25): Primer 39854_r_at
ggaaccctgcctgagacgcctccat

SEQ ID NO: 102 - DNA, 25 bp, Homo sapiens
  primer_bind (1)...(25): Primer 39854_r_at
gagacgcctccattaccactgcgca

SEQ ID NO: 103 - DNA, 25 bp, Homo sapiens
  primer_bind (1)...(25): Primer 39854_r_at
acgcctccattaccactgcgcagtg

SEQ ID NO: 104 - DNA, 25 bp, Homo sapiens
  primer_bind (1)...(25): Primer 39854_r_at
ccactgcgcagtgagatgaggggac

SEQ ID NO: 105 - DNA, 25 bp, Homo sapiens
  primer_bind (1)...(25): Primer 39854_r_at
aggggactcacagttgccaagaggg

SEQ ID NO: 106 - DNA, 25 bp, Homo sapiens
  primer_bind (1)...(25): Primer 39854_r_at
actcacagttgccaagaggggtctt

SEQ ID NO: 107 - DNA, 25 bp, Homo sapiens
  primer_bind (1)...(25): Primer 39854_r_at
cctcccctgggccgctgaggccccg

SEQ ID NO: 108 - DNA, 25 bp, Homo sapiens
  primer_bind (1)...(25): Primer 39854_r_at
gtgctgcccgagcacctcccccgcc

SEQ ID NO: 109 - DNA, 25 bp, Homo sapiens
  primer_bind (1)...(25): Primer 39854_r_at
gaactttgcagctgcccttccctcc

SEQ ID NO: 110 - DNA, 25 bp, Homo sapiens
  primer_bind (1)...(25): Primer 39854_r_at
tttgcagctgcccttccctccccgt

SEQ ID NO: 111 - DNA, 25 bp, Homo sapiens
  primer_bind (1)...(25): Primer 39854_r_at
agaattatttattttcgccaaagca

SEQ ID NO: 112 - DNA, 25 bp, Homo sapiens
  primer_bind (1)...(25): Primer 220757_s_at
gggctgcgcaagtacaactacacgc

SEQ ID NO: 113 - DNA, 25 bp, Homo sapiens
  primer_bind (1)...(25): Primer 220757_s_at
gcactttctacgctcgggagcggct

SEQ ID NO: 114 - DNA, 25 bp, Homo sapiens
  primer_bind (1)...(25): Primer 220757_s_at
tgcagagcgactggctgccttttga

SEQ ID NO: 115 - DNA, 25 bp, Homo sapiens
  primer_bind (1)...(25): Primer 220757_s_at
ggcctcgggagggcagaagctgtcc

SEQ ID NO: 116 - DNA, 25 bp, Homo sapiens
  primer_bind (1)...(25): Primer 220757_s_at
tgtccgaggacgagaacctggcctt

SEQ ID NO: 117 - DNA, 25 bp, Homo sapiens
  primer_bind (1)...(25): Primer 220757_s_at
acctggccttgaacgagtgcgggct

SEQ ID NO: 118 - DNA, 25 bp, Homo sapiens
  primer_bind (1)...(25): Primer 220757_s_at
agctcctgtcagccatcgagaagct

SEQ ID NO: 119 - DNA, 25 bp, Homo sapiens
  primer_bind (1)...(25): Primer 220757_s_at
aaaagcagggttggcctcagccctg

SEQ ID NO: 120 - DNA, 25 bp, Homo sapiens
  primer_bind (1)...(25): Primer 220757_s_at
acctctggaaatacttggctctgcc

SEQ ID NO: 121 - DNA, 25 bp, Homo sapiens
  primer_bind (1)...(25): Primer 220757_s_at
gccccatgggcacgggaggggcgcc

SEQ ID NO: 122 - DNA, 25 bp, Homo sapiens
  primer_bind (1)...(25): Primer 220757_s_at
agccgtggagctgtggaattgggcc

SEQ ID NO: 123 - DNA, 25 bp, Homo sapiens
  primer_bind (1)...(25): Primer 210075_at
cagtatgaatgctgggctctccgga

SEQ ID NO: 124 - DNA, 25 bp, Homo sapiens
  primer_bind (1)...(25): Primer 210075_at
agaggtagctggtgataccctgtcc

SEQ ID NO: 125 - DNA, 25 bp, Homo sapiens
  primer_bind (1)...(25): Primer 210075_at
ggaaggacttccacttcaacacttc

SEQ ID NO: 126 - DNA, 25 bp, Homo sapiens
  primer_bind (1)...(25): Primer 210075_at
gcacggcctgaacgcttcttaggcc

SEQ ID NO: 127 - DNA, 25 bp, Homo sapiens
  primer_bind (1)...(25): Primer 210075_at
ttaggccaagagacaccatgcggag

SEQ ID NO: 128 - DNA, 25 bp, Homo sapiens
  primer_bind (1)...(25): Primer 210075_at
catgcggagcctagtctgtgatcct

SEQ ID NO: 129 - DNA, 25 bp, Homo sapiens
  primer_bind (1)...(25): Primer 210075_at
gacatggtcctgagctctggacgga

SEQ ID NO: 130 - DNA, 25 bp, Homo sapiens
  primer_bind (1)...(25): Primer 210075_at
tgtggccggtgtatcaagggcgccc

SEQ ID NO: 131 - DNA, 25 bp, Homo sapiens
  primer_bind (1)...(25): Primer 210075_at
ttccagcaagcttcttgcgcttctc

SEQ ID NO: 132 - DNA, 25 bp, Homo sapiens
  primer_bind (1)...(25): Primer 210075_at
ctggcaccctcgactttatataaaa

SEQ ID NO: 133 - DNA, 25 bp, Homo sapiens
  primer_bind (1)...(25): Primer 210075_at
tgcactgcgtttcaaaaacccaccc

SEQ ID NO: 134 - DNA, 25 bp, Homo sapiens
  primer_bind (1)...(25): Primer 209018_s_at
ggcggaaacggctgtctgatggccc

SEQ ID NO: 135 - DNA, 25 bp, Homo sapiens
  primer_bind (1)...(25): Primer 209018_s_at
ggctgatgcctgggcagtgggagcc

SEQ ID NO: 136 - DNA, 25 bp, Homo sapiens
  primer_bind (1)...(25): Primer 209018_s_at
gagccatcgcctatgaaatcttcgg

SEQ ID NO: 137 - DNA, 25 bp, Homo sapiens
  primer_bind (1)...(25): Primer 209018_s_at
agccgcagctaccaagaggctcagc

SEQ ID NO: 138 - DNA, 25 bp, Homo sapiens
  primer_bind (1)...(25): Primer 209018_s_at
ctacctgcactgcccgagtcagtgc

SEQ ID NO: 139 - DNA, 25 bp, Homo sapiens
  primer_bind (1)...(25): Primer 209018_s_at
tcagtgcctccagacgtgagacagt

SEQ ID NO: 140 - DNA, 25 bp, Homo sapiens
  primer_bind (1)...(25): Primer 209018_s_at
tctgcccgagtagccgcaaatgtgc

SEQ ID NO: 141 - DNA, 25 bp, Homo sapiens
  primer_bind (1)...(25): Primer 209018_s_at
aatgtgcttcatctaagcctctggg

SEQ ID NO: 142 - DNA, 25 bp, Homo sapiens
  primer_bind (1)...(25): Primer 209018_s_at
ccaacaatcggccgccactttgttg

SEQ ID NO: 143 - DNA, 25 bp, Homo sapiens
  primer_bind (1)...(25): Primer 209018_s_at
atgctctttctggctaacctggagt

SEQ ID NO: 144 - DNA, 25 bp, Homo sapiens
  primer_bind (1)...(25): Primer 209018_s_at
gatgtccctgcatggagctggtgaa

SEQ ID NO: 145 - DNA, 25 bp, Homo sapiens
  primer_bind (1)...(25): Primer 210740_s_at
cccatcaccttggcagcaaagcact

SEQ ID NO: 146 - DNA, 25 bp, Homo sapiens
  primer_bind (1)...(25): Primer 210740_s_at
tgctgggtgagaggcatcagccccc

SEQ ID NO: 147 - DNA, 25 bp, Homo sapiens
  primer_bind (1)...(25): Primer 210740_s_at
atcagcccccacaagtatgtttttg

SEQ ID NO: 148 - DNA, 25 bp, Homo sapiens
  primer_bind (1)...(25): Primer 210740_s_at
aagtgctgagtgtcccgagagaggc

SEQ ID NO: 149 - DNA, 25 bp, Homo sapiens
  primer_bind (1)...(25): Primer 210740_s_at
cagctgggctgcaggatgcccactt

SEQ ID NO: 150 - DNA, 25 bp, Homo sapiens
  primer_bind (1)...(25): Primer 210740_s_at
ccatcagaactgcccggcttttttg

SEQ ID NO: 151 - DNA, 25 bp, Homo sapiens
  primer_bind (1)...(25): Primer 210740_s_at
actgaggacccaacaactaaccacg

SEQ ID NO: 152 - DNA, 25 bp, Homo sapiens
  primer_bind (1)...(25): Primer 210740_s_at
cacgacttgagttttgaaccccgat

SEQ ID NO: 153 - DNA, 25 bp, Homo sapiens
  primer_bind (1)...(25): Primer 210740_s_at
attaatgtctgtacgtcacctttcc

SEQ ID NO: 154 - DNA, 25 bp, Homo sapiens
  primer_bind (1)...(25): Primer 210740_s_at
aacaggaaagcgtggctggcctctt

SEQ ID NO: 155 - DNA, 25 bp, Homo sapiens
  primer_bind (1)...(25): Primer 210740_s_at
tcttgcactgctttgtctccaaaat

(19) United States
(12) Patent Application Publication
(10) Pub. No.: US 2007/0218513 A1
(43) Pub. Date: Sep. 20, 2007

(54) Differentially Expressed Genes Related to Coronary Artery Disease

(75) Inventors: Salah-Dine Chibout, Tagolsheim (FR); Peter Grass, Schopfheim (DE); Jacky Vonderscher, Cambridge, MA (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 10/575,814

(22) PCT Filed: Oct. 15, 2004
(86) PCT No.: PCT/EP04/11651

§ 371 Date: Jun. 7, 2007

Related U.S. Application Data
(60) Provisional application No. 60/511,784, filed on Oct. 16, 2003.
(60) Provisional application No. 60/574,818, filed on May 27, 2004.

(51) Int. Cl. G01N 33/48; G01N 33/563

(52) U.S. Cl. 435/7.24; 436/512; 436/86

(57) ABSTRACT

The present invention relates to genes whose expression is correlated to the prevalence of coronary artery disease. In particular, the invention relates to methods of prognosis, diagnosis and methods of monitoring coronary artery disease based on the measurement of gene expression. In addition, the present invention relates to methods of screening compounds for use in treatment of coronary artery disease as well as kits and arrays for use in identifying coronary artery disease.

### FIELD OF THE INVENTION

The present invention relates to genes whose expression is correlated to the prevalence of coronary artery disease. In particular, the invention relates to methods of identifying, predicting and monitoring coronary artery disease in a subject based on measurement of gene expression. In addition, the present invention relates to methods of screening compounds for use in treatment of coronary artery disease as well as kits for use in identifying coronary artery disease.

### BACKGROUND OF THE INVENTION

Coronary artery disease is the principal cause of death in the United States, Europe and most of Asia. As a multigenic disease, understanding patterns of gene expression may help to explain the individual differences :in susceptibility to the disease.

According to the present invention, coronary artery disease is defined as is a narrowing of the coronary arteries that supply blood and oxygen to the heart. Coronary disease usually results from the build up of fatty material and plaques (atherosclerosis). As a result of coronary artery stenosis, the flow of blood to the heart can slow or stop. The disease can be characterized by symptoms, including but not limited to, chest pain (stable angina), shortness of breath, atherosclerosis, ischemia/reperfusion, hypertension, restenosis and arterial inflammation.

In spite of the revolution in genomic knowledge, definitive and reproducible insights into how genetic variants relate to coronary disease are lacking. Defining how gene expression products, in the form of an entire population of e.g. mRNA, cDNA or proteins, relate to disease state provides an opportunity to gain new insights into disease.

Systemic and local inflammation plays a prominent pathogenetic role in atherosclerotic coronary artery disease (CAD), but the relationship of phenotypic changes in a sample isolated from a subject such blood, plasma or circulating leukocytes and the extent of CAD remains unclear. Thus, there is a need in the art for an understanding of whether gene expression patterns in such sample are associated with presence and extent of CAD.

### SUMMARY OF THE INVENTION

The present invention relates to genes which are differentially expressed in subjects with coronary artery disease relative to their expression in normal or non-disease states. As such they can be used as biomarkers for coronary artery disease. Further, these identified genes may act via their gene expression products with other genes involved in coronary artery disease. Further, the invention relates to proteins whose abundance is altered in subjects with CAD.

Methods are provided for identifying coronary artery disease in a subject, for identifying subjects who may be predisposed to coronary artery disease and for monitoring the treatment and progression of coronary artery disease. Further provided are methods of screening agents for use in the treatment of coronary artery disease. The present invention also provides a kit that can be used for identifying and monitoring coronary artery disease. Measurement of the biomarkers of the present invention can provide information that may correlate with a diagnosis of coronary artery disease.

While these biomarkers are identified from blood as described in the examples, the sample from which they may be detected is not limited to blood but may be detected in other types of samples such as serum, plasma, lymph, urine, tear, saliva, cerebrospinal fluid, or tissue.

A systematic and comprehensive approach has identified a large number of gene expression products such as mRNA or proteins that are differentially displayed in populations with and without coronary disease. These gene expression products include inflammatory mediators and defense mechanism proteins and mRNAs encoding them.

The simultaneous expression pattern of eight genes (Table 7), of 19 genes (Table 9), of 15 genes (Table 10), and/or the abundance of 11 Disease>Control and Predominant in Disease peptides and/or the abundance of 4 Control>Disease and Predominant in Control peptides (Table I1i) is highly predictive for coronary artery disease (CAD). Blood, plasma or peripheral leukocyte gene expression pattern is a thus non-invasive biomarker for coronary artery disease and leads to new pathophysiologic insights.

In one aspect of the invention, a method of identifying or predicting the predisposition of coronary artery disease in a subject is provided, comprising:

(i) determining the level of gene expression of at least one gene selected from Table 6 in a subject to provide a first value,

(ii) determining the level of gene expression of said at least one gene selected from Table 6 in a control or reference standard to provide a second value and

(iii) comparing whether there is a difference between said first value and second value.

In another embodiment of the invention, a method of identifying or predicting the predisposition of coronary artery disease in a subject -is provided, comprising the steps of (i) determining the level of gene expression of at least one gene selected from Table 7 and/or Table 9 and/or Table 10 in a subject to provide a first value, (ii) determining the level of gene expression of said at least one gene selected from Table 7 and/or Table 9 and/or Table 10 in a control or reference standard to provide a second value and (iii) comparing whether there is a difference between said first value and second value. Preferably, the control or reference standard is determined from a subject or group of subjects without coronary artery disease, wherein if the level of gene expression in the subject being tested (first value) -is higher than that of the control or reference standard (second value) it is indicative of the presence or prediction of coronary artery disease.

Another aspect of the invention provides a method of identifying or predicting coronary artery disease (CAD) in a subject wherein said method comprises the steps of (a) determining the level of one or more peptide selected from Table 11 in a subject to provide a first value, (b) determining the level of said one or more peptide selected from Table 11 in a control or reference standard to provide a second value and (c) comparing whether there is a difference between said first value and second value. In one embodiment, wherein the first value is increased for the one or more Disease>Control peptide and/or Predominant in Disease peptide of Table 11, and/or wherein the first value is decreased for the one or more Control>Disease and/or the Predominant in Control peptides of Table 11 it is an identification or prediction of coronary artery disease.

In another embodiment of the invention a method of identifying or predicting coronary artery disease (CAD) in a subject is provided comprising step (a) determining the level of one or more peptides selected from Table 11 and the level of gene expression of at least one gene selected from Table 6 and/or Table 7 and/or Table 9 and/or Table 10 in a subject to provide a first value, step (b) determining the level of said one or more peptide selected from Table 11 and the level of gene expression of said at least one gene selected from Table 6 and/or Table 7 and/or Table 9 and/or Table 10 in a control or reference standard to provide a second value and step (c) determining whether there is a difference between said first value and second value, wherein an elevated peptide level in the first value for the one or more Disease>Control and/or Predominant in Disease peptide of Table 11 and/or wherein a decrease of peptide level in the first value for the one or more Control>Disease and/or Predominant in Control peptide of Table 11 and wherein an elevated level of gene expression of the at least one gene selected from Table 6 and/or Table 7 and/or Table 9 and/or Table 10 in the first value is an identification or prediction of coronary artery disease.

In other embodiments of the invention the prediction of the presence of coronary artery disease has a probability of at least 50%, at least 60%, at least 75%, at least 90% or at least 95% chance.

In another embodiment of the invention, the level of gene expression and/or the level of a peptide may be expressed either as an absolute amount (e.g. ug/ml) or a relative amount (e.g. relative intensity of signals) where there may be a fold increase of level of gene expression and/or peptide level in one sample compared to another sample. In preferred embodiments of the invention, there is at least 20% greater difference in the first value compared to the second value.

In other preferred embodiments of the invention, the level of gene expression of at least one gene is selected from the group of genes:

(i) with the accession codes: BG537190, L37033, AL581768, AF055000, NM025241, AF151074, AF279372 and BF432478 (Table 6) or

(ii) with sequence numbers: SEQ ID No. 1, SEQ ID No. 2, SEQ ID No. 3, SEQ ID No. 4, SEQ ID No. 5, SEQ ID No. 6, SEQ ID No.7 and SEQ ID No. 8 (Table 7) is determined. In a further preferred embodiment the level of gene expression of a plurality of genes selected from Table 7 is determined. In another preferred embodiment the level of gene expression of at least the gene corresponding to SEQ ID No. 1 from Table 7 is determined. In yet another preferred embodiment the level of gene expression of at least two to eight genes selected from Table 7 is determined. Preferably, the levels of gene expression of 2, 3, 4, 5, 6, and/or 7 genes of Table 7 are determined. In the most preferred embodiment the level of gene expression of all eight genes selected from Table 7 is determined.

In another embodiment of the invention the level of gene expression of at least one gene selected from the group of genes consisting of: PMS2L5 (SEQ ID NO. 9), RXRA (SEQ ID NO. 10), GCN5L1 (SEQ ID NO. 11), CABIN1 (SEQ ID NO. 12), LGALS9 (SEQ ID NO. 13), CEBPA (SEQ ID NO. 14), LRRN4 (SEQ ID NO. 15), STXBP2 (SEQ ID NO. 16), SH3BP2 (SEQ ID NO. 17), RNF24 (SEQ ID NO. 18), PLAUR (SEQ ID NO. 19), RIS1 (SEQ ID NO. 20), ADD1 (SEQ ID NO. 21), GPSM3 (SEQ ID NO. 22), BC002942 (SEQ ID NO. 23), TNFRSF5 (SEQ ID NO. 24), N4BP1 (SEQ ID NO. 25), FLJ12438 (SEQ ID NO. 26) and MMP24 (SEQ ID NO. 27) of Table 9 is determined. In a preferred embodiment, the level of gene expression of the at least one gene corresponding to PMS2L5 (SEQ ID NO. 9) and/or RNF24 (SEQ ID NO. 18) from Table 9 is determined. In yet another preferred embodiment, the levels of gene expression of at least two to nineteen genes selected from Table 9 are determined. Preferably, the levels of gene expression of 2, 3, 4, 5, 6, 7, 8, 9, 10, 11, 12, 13, 14, 15, 16, 17 and/or 18 genes of Table 9 are determined. In a most preferred embodiment, the levels of gene expression of a plurality of genes selected from Table 9 are determined. In an even more preferred embodiment the levels of gene expression of all nineteen genes selected from Table 9 are determined.

In a further embodiment the level of gene expression of at least one gene selected from the group of genes consisting of: PTP4A1 (SEQ ID NO. 28), PAFAH1 B1 (SEQ ID NO. 29), SOX4 (SEQ ID NO. 30), ASNA1 (SEQ ID NO. 31), MAN2A2 (SEQ ID NO. 32), NFYC (SEQ ID NO. 33), NOTCH2 (SEQ ID NO. 34), HDAC5 (SEQ ID NO. 35), HCFC1 (SEQ ID NO. 36), NFX1 (SEQ ID NO. 37), CRSP2 (SEQ ID NO. 38), ICAM1 (SEQ ID NO.39), PSG3 (SEQ ID NO. 40), STC2 (SEQ ID NO. 41) and SEMA3C (SEQ ID NO. 42) of Table 10 is determined. Preferably, the level of gene expression of the gene corresponding to PTP4A1 (SEQ ID NO. 28) and/or MAN2A2 (SEQ ID NO. 32) from Table 10 is determined. More preferred, the levels of gene expression of at least two to fifteen genes selected from Table 10 are determined. Preferably, the levels of gene expression of 2, 3, 4, 5, 6, 7, 8, 9, 10, 11, 12, 13 and/or 14 genes of Table 10 are determined. In a most preferred embodiment, the levels of gene expression of a plurality of genes selected from Table 10 are determined. In an even more preferred embodiment the levels of gene expression of all fifteen genes selected from Table 10 are determined.

According to another embodiment of the invention the levels of gene expression of at least one gene selected from the genes of Table 7 and/or of at least one gene of Table 9 and/or of at least one gene selected of and/or Table 10 are determined. In another embodiment levels of gene expression of at least one gene selected from the genes of Table 9 and of at least one gene selected from Table 10 are determined. Preferably, the levels of gene expression of 2, 3, 4, 5, 6 or 7 genes of Table 7 and/or the levels of gene expression of 2, 3, 4, 5, 6, 7, 8, 9, 10, 11, 12, 13, 14, 15, 16, 17 or 18 genes of Table 9 and/or the levels of gene expression of 2, 3, 4, 5, 6, 7, 8, 9, 10, 11, 12, 13 or 14 genes of Table 10 are determined. In a most preferred embodiment, the levels of gene expression of a plurality of genes selected from Table 7 and/or Table 9 and/or Table 10 are determined. In an even more preferred embodiment the levels of gene expression of all genes selected from Table 7 and/or Table 9 and/or Table 10 are determined. The determination of the levels of gene expression of two or more genes may be performed separately or sequentially.

In other preferred embodiments of the invention, the level of 2, 3, 4, 5, 6, 7, 8 and/or 9 Disease>Control peptides and/or the level of 2 Predominant in Disease peptides and/or the level of 2 or 3 Control>Disease peptides and/or the Predominant in Control peptide from Table 11 are determined. Most preferably, the levels of a plurality or all Disease>Control peptides and/or all Predominant in Disease peptides and/or all Control>Disease peptides and/or all Predominant in Control peptide from Table 11 are determined. In most preferred embodiments said peptide levels are measured in a blood, plasma or serum sample. The determination of the levels of gene expression of two or more genes and/or of peptide levels may be performed separately or sequentially.

In another preferred embodiment of the invention the levels of gene expression of at least 5, 10, 15, 20, 25, 30, 40, 50, 60, 70, or at least 80, 90, 100, or at least 110, 120, 140 or 150 genes selected from the genes of Table 6 are determined. In another embodiment the level of gene expression for a plurality of genes selected from Table 6 are determined and in a further embodiment the level of expression of all 160 genes of Table 6 is determined.

According to the present invention, the determination of the level of gene expression comprises measuring the protein expression product and detection of said protein expression product may be made by using a antibody, antibody derivative or antibody fragment which specifically binds to the protein.

In another embodiment of the invention, the determination of the level of gene expression or of the peptide level comprises measuring the gene expression of a transcribed polynucleotide of the gene encoding a peptide wherein the transcribed polynucleotide may be mRNA or cDNA. Accordingly, the level of expression may be detected by microarray analysis, Northern blot analysis, reverse transcription PCR or RT-PCR.

In a further aspect of the invention, the level of gene expression and/or the peptide level may be measured ex vivo in a sample selected from the group of: blood, serum, plasma, lymph, urine, tear, saliva, cerebrospinal fluid, -leukocyte sample or tissue sample.

In yet another embodiment of the invention, CAD-Index may be measured wherein a CAD Index between 23-100 is indicative of the probability of the presence of coronary artery disease.

In another aspect of the invention, a method of monitoring a subject identified as having coronary artery disease before and after treatment is provided, comprising: (i) determining the level of gene expression of at least one gene from Table 6 or Table 7 in said subject prior to treatment providing a first value, (ii) determining the level of gene expression of the same gene after treatment ,providing a second value and (iii) comparing the difference in the level of gene expression of said subject before treatment and after treatment. Another aspect of the invention provides a method of monitoring a subject identified as having coronary artery disease before and during and/or after treatment comprising step (i) determining the level of gene expression of at least one gene from Table 9 or Table 10 in said subject prior to treatment providing a first value, step (ii) determining the level of gene expression of the same at least one gene as in (i) during and/or after treatment providing a second value and (iii) comparing comparing the first value with the second value. Another embodiment provides a method of monitoring a subject identified as having coronary artery disease before and during/after treatment based on the analysis of expression of at least one gene selected of Table 7 and/or Table 9 and/or Table 10. A further embodiment of the invention relates to the use of determining the level of one or more peptides selected from Table 11 in said method. And a still further embodiment of the invention provides such method based on determining the level of one or more peptides selected from Table 11 and the level of gene expression of at least one gene from Table 6 or Table 7 and/or Table 9 and/or Table 10.

In one embodiment of the invention, the method of monitoring a subject further comprises: (iv) determining that a difference in the level of gene expression corresponds to the efficacy of the treatment of coronary artery disease in said subject. In a preferred embodiment of the invention, a positive response to the treatment is measured when level of gene expression decreases. In another preferred embodiment a decrease in peptide level in the second value for the one or more Disease>Control and/or Predominant in Disease peptide of Table 11; and/or an increase in peptide level in the second value for the one or more Control>Disease and/or Predominant in Control peptide of Table 11, and/or a decrease of level of gene expression of the at least one gene selected from Table 6 or Table 7 and/or Table 9 and/or Table 10 in the second value is indicative for a positive response to the treatment.

In a preferred embodiment of the invention, the level of gene expression of at least SEQ ID No.1 is determined. In another preferred embodiment of the invention, the level of gene expression of a plurality of genes in Table 7 is determined. In the most preferred embodiment of the :invention, the level of gene expression of all 8 genes listed in Table 7 is determined. In other preferred embodiments of the invention the variety of combinations of the one or more peptides of Table 11 and/or of the at least one gene of Table 6 and/or Table 7 and/or Table 9 and/or Table 10 are determined as described further above.

In another aspect of the invention, a method of monitoring the progression or severity of coronary artery disease is provided, comprising:

(i) determining the level of gene expression of at least one gene from Table 6 or Table 7 at an initial time point providing a first value,

(ii) determining the level of gene expression of at least one gene from Table 6 or Table 7 at a time point after the initial time point providing a second value,

(iii) comparing the difference in the level of gene expression of the first value to the second value wherein a higher level of gene expression in the second value is indicative of an increase in severity of coronary artery disease. Another embodiment of the invention provides a method of monitoring the progression or severity of coronary artery disease comprising the steps of (i) determining the level of gene expression of at least one gene from Table 9 or Table 10 at an initial time point providing a first value, (ii) determining the level of gene expression of the same at least one gene as in (i) at a time point after the initial time point providing a second value, and (iii) determining the difference in the level of gene expression of the first value to the second value wherein a higher level of gene expression in the second value is indicative of an increase in severity of coronary artery disease. In a further embodiment said method is based on the determining the level of gene expression of at least one gene from Table 7 and/or Table 9 and/or Table 10. In preferred embodiments of the invention, a lower level of gene expression in the second value is indicative of a decrease in severity of coronary artery disease. A still further embodiment of the invention provides a method of monitoring the progression or severity of coronary artery disease comprising step (i) determining the level of one or more peptide selected from Table 11 and/or the level of gene expression of at least one gene from Table 6 or Table 7 and/or Table 9 and/or Table 10 at an initial time point providing a first value, step (ii) determining the level of the one or more peptide and the level of gene expression of the same genes as in (i) at a time point after the initial time point providing a second value, and step (iii) determining the difference in the level of the one or more peptides and gene expression of the first value to the second value wherein a higher level of the one or more Disease>Control and/or Predominant in Disease peptide or a lower level of the Control>Disease peptides or the Predominant in Control peptides of Table 11 in the second value, and/or a higher level of expression of the at least one gene from Table 6 or Table 7 and/or Table 9 and/or Table 10 in the second value is indicative of an increase in severity of coronary artery disease.

In yet another aspect of the invention, a method of screening candidate agents for use in treatment of coronary artery disease is provided, comprising:

(i) contacting a cell capable of expressing a gene selected from Table 6 or Table 7 with a candidate agent ex vivo,

(ii) determining the level of gene expression of said at least one gene from Table 6 or Table 7 to provide a first value,

(iii) determining the level of gene expression of the same at least one gene from Table 6 or Table 7 in a sample in the absence of the candidate agent to provide a second value, and

(iv) comparing the first value with the second value wherein a difference in level of gene expression is indicative of an agent potentially capable of being used for the treatment of coronary artery disease. Another aspect of the invention provides a method of screening candidate agents for use in treatment of coronary artery disease said method comprises the steps of (i) contacting a cell or sample of cells capable of expressing a gene selected from Table 7 and/or Table 9 and/or Table 10 with a candidate agent ex vivo, (ii) determining the level of gene expression of said at least one gene from Table 7 and/or Table 9 and/or Table 10 to provide a first value, (iii) determining the level of gene expression of the same at least one gene from Table 7 and/or Table 9 and/or Table 10 in a cell or sample of cells in the absence of the candidate agent to provide a second value, and (iv) comparing the first value with the second value wherein a difference in level of gene expression is indicative of an agent potentially capable of being used for the treatment of coronary artery disease. Another embodiment of the invention provides the following steps of (i) contacting a cell or sample of cells capable of producing at least one peptide selected from Table 11 and/or capable of expressing at least one gene selected from Table 6 or Table 7 and/or Table 9 and/or Table 10 with a candidate agent ex vivo, (ii) determining the level of the one or more peptide of (i) and/or the level of gene expression of the at least one gene of (i) to provide a first value, (iii) determining the level of the one or more peptide of (i) and/or the level of gene expression of the at least one gene of (i) in a cell or sample of cells in the absence of the candidate agent to provide a second value, and (iv) comparing the first value with the second value wherein a difference in the level of the one or more peptide and/or in the level of gene expression is indicative of an agent potentially capable of being used for the treatment of coronary artery disease. Preferably, a decrease in the first value of the at least one Disease>Control peptide and/or Predominant in Disease peptide and/or an increase in the first value of the at least one Control>Disease and/or Predominant in Control peptide from Table 11 and/or a decrease in the first value of the at least one gene selected from Table 6 or Table 7 and/or Table 9 and/or Table 10 is indicative of an agent potentially capable of being used for the treatment of coronary artery disease.

In a preferred embodiment of the invention, there is decrease in the level of gene expression in the presence of a candidate agent. In yet another preferred embodiment, a decrease in the level of gene expression of at least SEQ ID No. 1 may be measured. In a further preferred embodiment, a decrease in the level of gene expression of a plurality of genes selected from Table 7 can be measured. In a most preferred embodiment, a decrease in the level of gene expression of all eight genes selected from Table 7 may be measured. In another preferred embodiment the level of gene expression of at least SEQ ID No. 9 of Table 9 and/or of at least SEQ ID No. 28 of Table 10 the is determined. Most preferably, the level of a plurality of peptides of Table 11 and/or the levels of gene expression of a plurality of genes selected from Table 7 and/or Table 9 and/or Table 10 are determined.

In another aspect of the invention, a method of treating or preventing coronary artery disease is provided, comprising administering to a subject an effective amount of an agent that can induce a decrease in the level of gene expression, synthesis, or activity of at least one gene or gene expression products from Table 6 or Table 7. In another aspect of the invention a method of treating or preventing coronary artery disease is provided said method comprises administering to a subject an effective amount of an agent that can induce a decrease in the level of gene expression, synthesis, or activity of at least one gene or gene expression products from Table 6 or Table 7 and/or Table 9 and/or Table 10. Further is provided, a method of treating or preventing coronary artery disease comprising administering to a subject an effective amount of an agent that can induce a decrease in the level of at least one Disease>Control and/or Predominant in Disease peptide and/or an increase in the level of at least one Control>Disease and/or Predominant in Control peptide from Table 11 and/or a decrease in gene expression, synthesis, or activity of at least one gene or gene expression products from Table 6 or Table 7 and/or Table 9 and/or Table 10. In a preferred embodiment said agent is selected from the group consisting of antisense oligonucleotides, double stranded RNA, ribozyme, small molecule, antibody or antibody fragment.

In yet another aspect of the invention, a method of manufacture of a medicament for the treatment or prevention of coronary artery disease is provided, comprising an effective amount of an agent that can induce a decrease in the level of gene expression, synthesis, or activity of at least one gene or gene expression products from Table 6 or Table 7.

Another aspect of the invention relates to the use of a substance comprising an effective amount of an agent that can induce a decrease in the level of gene expression, synthesis, or activity of at least one gene or gene expression products from Table 6 or Table 7 and/or Table 9 and/or Table 10 in the manufacture of a medicament for the treatment or prevention of coronary artery disease. Another aspect provides the use of a substance comprising an effective amount of an agent that can induce a decrease in the level of at least one Disease>Control and/or Predominant in Disease peptide and/or induce an increase in the level of at least one Control>Disease and/or Predominant in Control peptide from Table 11 and/or a decrease in gene expression, synthesis, or activity of at least one gene or gene expression products from Table 6 or Table 7 and/or Table 9 and/or Table 10 in the manufacture of a medicament for the treatment or prevention of coronary artery disease.

In other preferred embodiments of the invention the level and the variety/combination of peptides of Table 11 and/or the level of gene expression and the variety of the genes of Table 6 and/or Table 7 and/or Table 9 and/or Table 10 are determined.

In the methods of screening candidate agents, the methods of monitoring the progression or severity of coronary artery disease, the methods of monitoring a subject identified as having coronary artery disease before and after treatment, the methods of treating or preventing coronary artery disease or the methods of manufacture of a medicament for the treatment or prevention of coronary artery disease provided by the invention, the level of peptides or gene expression may be determined for a variety/combination of peptides or genes as described further above for the method of identifying or predicting the predisposition of coronary artery disease. Accordingly, the levels of gene expression of 2, 3, 4, 5, 6 or 7 genes of Table 7 and/or the levels of gene expression of 2, 3, 4, 5, 6, 7, 8, 9, 10, 11, 12, 13, 14, 15, 16, 17 or 18 genes of Table 9 and/or the levels of gene expression of 2, 3, 4, 5, 6, 7, 8, 9, 10, 11, 12, 13 or 14 genes of Table 10 are determined. In a most preferred embodiment, the levels of gene expression of a plurality of genes selected from Table 6 or Table 7 and/or Table 9 and/or Table 10 are determined. In an even more preferred embodiment the levels of gene expression of all genes selected from Table 6 or Table 7 and/or Table 9 and/or 10 are determined. In other preferred embodiments of the invention, the level of 2, 3, 4, 5, 6, 7, 8 or 9 Disease>Control peptides and/or the level of 2 Predominant in Disease peptides and/or the level of 2 or 3 Control>Disease peptides and/or the Predominant in Control peptide from Table 11 are determined. Most preferably, the levels of all Disease >Control peptides and/or all Predominant in Disease peptides and/or all Control>Disease peptides and/or all Predominant in Control peptide from Table 11 are determined. Further embodiments provide that the level of peptide or gene expression of a plurality or all of said peptides of Table 11 and/or genes of Table 6 or Table 7 and/or Table 9 and/or Table 10 are determined.

In a further aspect of the invention, a kit is provided for the identifying or predicting the predisposition coronary artery disease in a subject comprising (i) instructions for determining the level of gene expression of at least one gene from Table 6 or Table 7 and (ii) control or reference standard level of gene expression from a normal subject or subjects without coronary artery disease for at least one gene in Table 6 or Table 7. In another aspect a kit for the identifying or predicting the predisposition coronary artery disease in a subject is provided, said kit comprising (i) instructions for determining the level of gene expression of at least one gene selected from Table 6 or Table 7 and/or Table 9 and/or Table 10, (ii) control or reference standard level of gene expression from a normal subject or subjects without coronary artery disease for the genes selected from Table 6 or Table 7 and/or Table 9 and/or Table 10 of (i). In one embodiment, the kit additionally contains antibodies, antibody derivatives or antibody fragments capable of binding to a polypeptide encoded by of at least one gene from Table 6 and/or Table 7, and/or Table 9 and/or Table 10. Further, the invention relates to a kit for the identifying or predicting coronary artery disease in a subject comprising (a) instructions for determining the peptide level of at least one peptide from Table 11 and (b) control or reference standard peptide level from a normal subject or subjects without coronary artery disease for at least one peptide in Table 11. In a preferred embodiment the kit further comprises (c) an antibody that binds to said at least one peptide from Table 11. Another embodiment of the invention provides a kit for the identifying or predicting coronary artery disease in a subject which comprises (a) instructions for determining the peptide level of at least one peptide from Table 11 and for determining the level of gene expression of at least one gene selected from Table 6 or Table 7 and/or Table 9 and/or Table 10 and (b) control or reference standard peptide level from a normal subject or subjects without coronary artery disease for at least one peptide in Table 11 and for determining the level of gene expression of at least one gene of Table 6 or Table 7 and/or Table 9 and/or Table 10, and optionally also (c) an antibody that binds to said at least one peptide from Table 11 and additionally antibodies, antibody derivatives or antibody fragments capable of binding to a polypeptide encoded by the at least one gene from Table 6 or Table 7, and/or Table 9 and/or Table 10 are provided. In further embodiments of the invention kits may be used in any one of the methods of the invention.

### BRIEF DESCRIPTION OF FIGURES

FIG. 1—illustrates the second degree polynomial regression analysis of the resulting t-scores versus CAD-index resulted in the prediction model including 95% confidence range of the regression and the 95% prediction interval with r²=0.764 (p<0.001) (A), and illustrates the predicted CAD-index versus the CAD-index of model 1 involving 8 predictor genes (B).

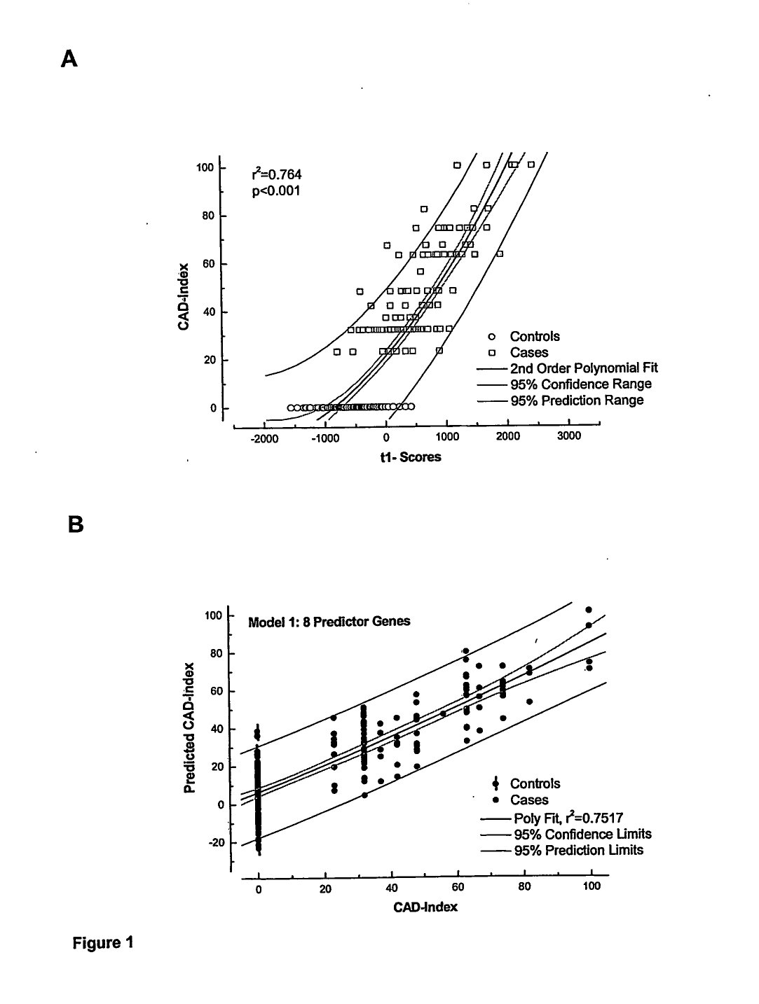

FIG. 2—illustrates the Variable Importance in the Projection (VIP) of each gene for the separate PLS analyses of the three different cohorts compared to the PLS analysis including all subjects. Displayed are the 24 genes with the highest VIP. The curve shows a steep decrease for the first 8 genes; thereafter, the decrease is rather flat and almost linear.

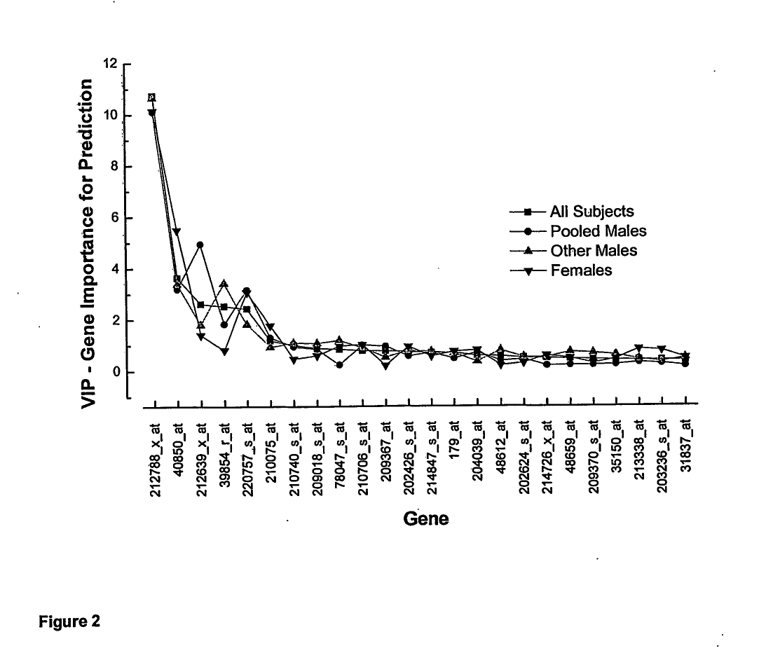

FIG. 3—illustrates the final PLS analysis with the eight most important predictor genes applied to all 222 subjects involved in this study. The subjects are ordered by their CAD index and have a CAD Index between 23-100 depending on the severity of stenosis. In order to better demonstrate the predictive power of the model, the CAD Index is superimposed on the t-scores.

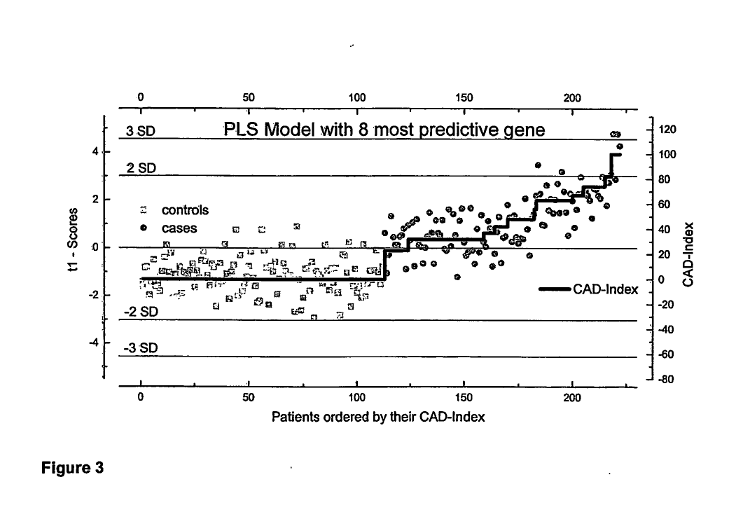

FIG. 4—illustrates the predicted CAD-index versus the CAD-Index of model 2 involving 19 predictor genes (A) and of model 3 involving 15 predictor genes (B).

### DETAILED DESCRIPTION OF THE INVENTION

Unless defined otherwise, all technical and scientific terms used herein have the meaning commonly understood by a person skilled in the art to which this invention belongs. The following references provide one of skill with a general definition of mi any of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Biomarker” in the context of the present invention refers to genes and gene expression products (i.e. proteins or polypeptides, mRNA) which are differentially expressed in a sample taken from subjects having coronary artery disease as compared to a comparable sample taken from control subjects (e.g., a person with a negative diagnosis or undetectable coronary artery disease, normal or healthy subject).

“Proteins or polypeptides or peptides” of the present invention are contemplated to include any fragments thereof, in particular, immunologically detectable fragments. One of skill in the art would recognize that proteins which are released by cell in the heart which become damaged during vascular injury could become degraded or cleaved into such fragments.

Additionally, certain proteins or polypeptides are synthesized in an inactive form, which may be subsequently activated by proteolysis. Such fragments of a particular protein may be detected as a surrogate for the protein itself.

The term “sample” as used herein refers to a sample from a subject obtained for the purpose of identification, diagnosis, prediction, or monitoring. In certain aspects of the invention such a sample may be obtained for the purpose of determining the outcome of an ongoing condition or the effect of a treatment regimen on a coronary artery disease. Preferred test samples include blood, serum, plasma, lymph, urine, tear, saliva, cerebrospinal fluid, leukocyte or tissue samples. In addition, one of skill in the art would realize that some test samples would be more readily analyzed following a fractionation or purification procedure, for example, separation of whole blood into serum or plasma components.

A difference in the “level of gene expression” or in the “peptide level” is a relative difference. For example, it may be a difference in the level of gene expression a sample taken from a subject having coronary artery disease as compared to control subjects or a reference standard. A comparison can be -made between the level of gene expression in a subject at risk of coronary artery disease to a subject known to be free of a given condition, i.e. “normal” or “control”. Alternatively, a comparison can be made to a “reference standard” known to be associated with a good outcome (e.g. the absence of coronary artery disease) such as an average level found in a population of normal individuals not suffering from coronary artery disease. According to the present invention, a comparison can be made between the level of gene expression and the identification or predisposition of a subject to develop coronary artery disease.

The level of gene expression or the level of proteins/peptides present in a sample being tested can be either in absolute amount (e.g. μg/ml) or a relative amount (e.g. relative intensity of signals).

A difference is present between the two samples if the amount of gene expression is statistically significantly different from the amount of the polypeptide in the other sample. For example, there is a difference in gene expression or in the level of proteins/peptides between the two samples if the amount of polypeptide is present in at least about 20%, at least about 30%, at least about 50%, at least about 80%, at least about 100%, at least about 200%, at least about 400%, at least about 600%, at least about 800%, or at least about 1000% greater than it is present in the other sample.

Identifying or predicting the predisposition of coronary artery disease may be considered as a diagnostic technique. Diagnostic methods differ in their sensitivity and specificity. The skilled artisan often makes a diagnosis, for example, on the basis of one or more diagnostic indicators, in the present invention these are the expression levels of the genes from Table 6 and/or Table 7 and/or Table 9 and/or Table 10, and/or the peptide levels of Table 11. The presence, absence, or amount of which is indicative of the presence, severity, or absence of the coronary artery disease.

Multiple determination of the gene expression of one or more genes can be made of Table 6 and/or Table 7 and/or Table 9 and/or Table 10, and/or of the peptide levels of Table 11 can be made as well as determination of a temporal change in gene expression or peptide abundancy which can be used to monitor the progress of the disease or a treatment of the disease. For example, gene expression/peptide abundancy may be determined at an initial time, and again at a second time. In such aspects, an increase in the gene expression and/or peptide level from the initial time to the second time may be diagnostic of coronary artery disease. Likewise, a decrease in the gene expression and/or peptide level from the initial time to the second time may be indicative of a responsiveness of a subject to a particular type of treatment of coronary artery disease. Furthermore, the change in gene expression of one or more genes may be related to the severity of coronary artery disease and future adverse events.

In one embodiment of the invention, the level of gene expression as least one gene from Table 6 is determined. In a preferred embodiment, the level of gene expression of at least SEQ ID. No. 1 (Table 7) is determined. In another preferred embodiment, the level of gene expression of a plurality of genes from Table 7 is determined. In a most preferred embodiment, the level of gene expression all eight genes of Table 7 are determined. In other preferred embodiments, the level of gene expression of at least SEQ ID. No. 9 (Table 9) and/or SEQ ID. No. 28 (Table 10) is determined. In yet other preferred embodiments the levels of gene expression of 2, 3, 4, 5, 6, 7 or 8 genes of Table 7 and/or the levels of gene expression of 2, 3, 4, 5, 6, 7, 8, 9, 10, 11, 12, 13, 14, 15, 16, 17, 18 or 19 genes of Table 9 and/or the levels of gene expression of 2, 3, 4, 5, 6, 7, 8, 9, 10, 11, 12, 13, 14 or 15 genes of Table 10 are determined. In a most preferred embodiment, the levels of gene expression of a plurality of genes selected from Table 6 or Table 7 and/or Table 9 and/or Table 10 are determined. In an even more preferred embodiment the levels of gene expression of all genes selected from Table 6 or Table 7 and/or Table 9 and/or 10 are determined. In other preferred embodiments of the invention, the level of 2, 3, 4, 5, 6, 7, 8 or 9 Disease>Control peptides and/or the level of 2 Predominant in Disease peptides and/or the level of 2 or 3 Control>Disease peptides and/or the Predominant in Control peptide from Table 11 are determined. Most preferably, the levels of all Disease>Control peptides and/or all Predominant in Disease peptides and/or all Control>Disease peptides and/or all Predominant in Control peptide from Table 11 are determined. The level of peptides may optionally be determined together, simultaneously or sequentially, with the level of gene expression of the genes of Table 6 or Table 7 and/or Table 9 and/or Table 10. Further embodiments provide that the level of peptide or gene expression of a plurality or all of said peptides of Table 11 and/or genes of Table 6 or Table 7 and/or Table 9 and/or Table 10 are determined.

The skilled artisan will understand that, while in certain aspects comparative measurements of gene expression are made of the same gene at multiple time points, one could also measure a given gene at one time point, and a second gene at a second time point, and a comparison of the gene expression of these genes may provide diagnostic information or monitor the progress of the disease.

In a preferred aspect of the invention, gene expression of one or more genes from Table 7 and/or Table 9 and/or Table 10 and/or level of peptides of Table 11 may be comparatively measured at different time points.

The phrase “probability of the presence of coronary artery disease” as used herein refers to methods by which the skilled artisan can predict the condition in a subject. It does not refer to the ability to predict the coronary artery disease with 100% accuracy. Instead, the skilled artisan will understand that it refers to an increased probability that a coronary artery disease is present or will develop. For example, coronary artery disease is more likely to occur in a subject having high levels of expression of genes of Table 6 and/or Table 7 and/or Table 9 and/or Table 10 and/or increased levels for Disease>Control peptide and/or Predominant in Disease peptide of Table 11, and/or decreased levels of Control>Disease and/or the Predominant in Control peptides of Table 11 when compared to a control or reference standard such as a subject not being affected by or having a predisposition for CAD. In one aspect of the invention, the probability of the presence of coronary artery disease is about a 50% chance, about a 60% chance, about a 75% chance, about a 90% chance, and about a 95% chance. The term “about” in this context refers to +/−1%.

The skilled artisan will understand that associating a particular gene with a predisposition to coronary artery disease is a statistical analysis. Additionally, a change in gene expression and/or peptide level from baseline levels may be reflective of patient prognosis, and the degree of change in gene expression may be related to the severity of adverse events. Statistical significance is often determined by comparing two or more populations, and determining a confidence interval and/or a p value. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.01, 0.005, 0.001, and 0.0001.

In a further aspect, the invention relates to kits for identification of coronary artery disease in a subject. These kits comprise devices and reagents for measuring gene expression and/or determining peptide levels in a subject's sample and instructions for performing the assay and interpreting the results. Such kits preferably contain sufficient reagents to perform one or more such determinations.

The “sensitivity” of an assay according to the present invention is the percentage of diseased individuals (those with coronary artery disease) who test positive (percent of “true positives”). Diseased individuals not detected by the assay are “false negatives”. Subjects who are not diseased and who test negative in the assay, are termed “true negatives.” The “specificity” of a diagnostic assay is 1 minus the false positive rate, where the “false positive rate” is defined as. the proportion of those without the disease who test positive. While a particular diagnostic method may not provide a definitive diagnosis of a condition, it suffices if the method provides a positive indication that aids in diagnosis.

Measurement of Gene Expression

Numerous methods and devices are well known to the skilled artisan for the detection and analysis of the gene expression and measurement of peptide levels of the present invention. The term “gene expression” refers to the presence or amount of a specific gene including, but not limited to, mRNA, CDNA or the polypeptide, peptide or protein expression product of a specific gene. In a preferred aspect of the invention, the gene expression of genes from Table 6 and/or Table 7 and/or Table 9 and/or Table 10 and/or the level of peptides of Table 11 are determined.

In one embodiment of the invention, the gene expression is determined by measuring RNA levels. Gene expression may be detected using a PCR-based assay. Or in other aspects of the invention, reverse-transcriptase PCR (RT-PCR) is used to detect the expression of RNA. In RT-PCR, RNA is enzymatically converted to cDNA using a reverse-transcriptase enzyme. The cDNA is then used as a template for a PCR reaction. PCR products can be detected by any suitable method including, but not limited to, gel electrophoresis and staining with a DNA-specific stain or hybridizabon to a labeled probe. In yet another aspect of the invention, the quantitative RT-PCR with standardized mixtures of competitive templates can be utilized.

In another embodiment of the present invention, gene expression is detected using a hybridization assay. In a hybridization assay, the presence or absence of biomarker is determined based on the ability of the nucleic acid from the sample to hybridize to a complementary nucleic acid molecule, e.g., an oligonucleotde probe. A variety of hybridization assays are available. In some embodiments of the invention, hybridization of a probe to the sequence of interest is detected directly by visualizing a bound probe, e.g., a Northern or Southern assay. In these assays, DNA (Southern) or RNA (Northern) is isolated. The DNA or RNA is then cleaved with a series of restriction enzymes that cleave infrequently in the genome and not near any of the markers being assayed. The DNA or RNA is then separated, e.g., on an agarose gel, and transferred to a membrane. A labeled probe or probes, e.g., by incorporating a radionucleotide, is allowed to contact the membrane under low-, medium- or high-stringency conditions. Unbound probe is removed and the presence of binding is detected by visualizing the labeled probe. In another embodiment of the invention, the gene expression is determined for the genes of Table 6 or Table 7 and/or Table 9 and/or Table 10 and/or for the genes encoding the peptides of Table 11.

In yet another embodiment of the invention, the gene expression is determined by measuring polypeptide gene expression products. In a preferred aspect of the invention, gene expression is measured by identifying the amount of one or more polypeptides encoded by one of the genes in Table 6 or Table 7 and/or Table 9 and/or Table 10 and/or the amount of peptides of Table 11. The present invention is not limited by the method in which gene expression is detected or measured.

A protein or polypeptide or peptide expression product encoded by one of the genes in Table 6 or Table 7 and/or Table 9 and/or Table 10 and/or a peptide of Table 11 may be detected by an suitable method. With regard to peptides, polypeptides or proteins in samples, immunoassay devices and methods are often used. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule.

The presence or amount of a protein or polypeptide or peptides is generally determined using specific antibodies and detecting specific binding. Any suitable immunoassay may be utilized, for example, enzyme-linked immunoassays (ELISA), radioimmunoassays (RIAs), competitive binding assays, and the like. Specific immunological binding of the antibody to the protein or polypeptide can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the proteins or polypeptides are also contemplated by the present invention. The antibodies can be immobilized onto a variety of solid supports, such as magnetic or chromatographic matrix particles, the surface of an assay place (such as microtiter wells), pieces of a solid substrate material (such as plastic, nylon, paper), and the like. An assay strip can be prepared by coating the antibody or a plurality of antibodies in an array on solid support. This strip can then be dipped into the test sample and then processed quickly through washes and detection steps to generate a measurable signal, such as a colored spot.

The analysis of a plurality of genes and/or peptides of the present invention may be carried out separately or simultaneously with one test sample. In addition, one skilled in the art would recognize the value of testing multiple samples (for example, at successive time points) from the same individual. Such testing of serial samples allows the identification of changes in gene expression and/or peptide levels over time. Increases or decreases in gene expression levels, as well as the absence of change in gene expression and/or peptide levels, can provide useful information about the disease status that includes, but is not limited to identifying the approximate time from onset of the event, the presence and amount of salvagable sample, the appropriateness of drug therapies, the effectiveness of various therapies as indicated by reperfusion or resolution of symptoms, differentiation of the various types of coronary artery disease, identification of the severity of the event, identification of the disease severity, and identification of the patient's outcome, including risk of future events.

A panel comprising of the genes referenced above may be constructed to provide relevant information related to the diagnosis or prognosis of coronary artery disease and management of subjects with coronary artery disease. Such a panel can be constructed preferably using the sequences of Table 7 and/or Table 9 and/or Table 10 and/or the peptides of Table 11. The analysis of a single genes or subsets of genes comprising a larger panel of genes alone or in combination with the analysis of a single peptide or a subset of peptdes can be carried out by one skilled in the art to optimize sensitivity or specificity.

The analysis of gene expression and/or determination of peptide levels can be carried out in a variety of physical formats as well. For example, the use of microtiter plates or automation could be used to facilitate the processing of large numbers of test samples in a high through-put manner.

In another aspect of the invention, an array is provided to which probes that correspond in sequence to gene products, e.g., cDNAs, mRNAs, cRNAs, polypeptides and fragments thereof, can be specifically hybridized or bound at a known position. In one embodiment of the invention, the array is a matrix in which each position represents a discrete binding site for a product encoded by a gene (e.g., a protein or RNA) preferably of the genes listed in Table 7 and/or Table 9 and/or Table 10 and/or of the peptides listed in Table 11. In another aspect of the invention, the “binding site”, hereinafter “site”, is a nucleic acid or nucleic acid analogue to which a particular cognate CDNA can specifically hybridize. The nucleic acid or analogue of the binding site can be, e.g., a synthetic oligomer, a full-length cDNA, a less than full-length CDNA or a gene fragment.

In another aspect, the present invention provides a kit for the analysis of gene expression and/or peptide levels. Such a kit preferably comprises devices and reagents for the analysis of at least one test sample and instructions for performing the assay. Optionally the kits may contain one or more means for converting gene expression and/or amounts of peptides to a diagnosis or prognosis of coronary artery disease in a subject. Comparison of the subject's gene expression pattern, with the controls or reference standards, would indicate whether the subject has coronary artery disease.

In one embodiment of the invention, the kits contain antibodies specific for at least one gene, preferably from Table 7 and/or Table 9 and/or Table 10 and/or for at least one peptide of Table 11. In other embodiments, the kits contain reagents specific for the detection of nucleic acid, e.g., oligonucleotde probes or primers. In preferred embodiments, the kits contain all of the components necessary to perform a detection assay, including all controls and instructions for performing assays and for analysis of results. In one embodiment of the invention, the kits contain instructions including a statement of intended use as required by the Environmental Protection Agency or U.S. Food and Drug Administration (FDA) for the labeling of in vitro diagnostic assays and/or of pharmaceutical or food products.

In another aspect of the present invention, a method of screening agents for use in the treatment of coronary artery disease is provided. In particular agents that can induce a decrease in the level of gene expression, synthesis or activity of at least one gene or gene expression product from Table 6 or Table 7 and/or Table 9 and/or Table 10 and/or induce a decrease in the level of at least one Disease>Control and/or Predominant in Disease peptide and/or induce an increase in the level of at least one Control>Disease and/or Predominant in Control peptide from Table 11.

For example, in one embodiment one would first treat a test subject known to have coronary artery disease with a test agent and then analyze a representative sample of the subject for the level of expression of the genes or sequences which change in expression in response to coronary artery disease and/or for the level of peptide. One then compares the analysis of the sample with a control known to have coronary artery disease but not given the test compound and thereby identifies test compounds that are capable of modifying the gene expression.

In another embodiment of the present invention, one would base a therapy on the sequences of the genes disclosed in Table 7 and/or Table 9 and/or Table 10 and/or Table 11. In general, one would try to -decrease the expression of genes identified herein as over-expressed in coronary artery disease, and to induce a decrease in the level of Disease>Control and/or Predominant in Disease peptide and to induce an increase in the level of Control>Disease and/or Predominant in Control identified herein.

Methods of decreasing the expression of said genes would be known to one of skill in the art. Examples for supplementation of expression would include supplying subject with additional copies of the gene. A preferred example for decreasing expression would include RNA antisense technologies or pharmaceutical intervention. The genes or peptides disclosed in Table 6 or Table 7 and/or Table 9 and/or Table 10 would be appropriate drug development targets, preferably those in Table 7 and 9 and 10 and/or Table 11.

The details of one or more embodiments of the invention are set forth in the accompanying description above. Although any methods and -materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

### EXAMPLES

The following EXAMPLES are presented in order to more fully illustrate the preferred embodiments of the invention. These examples should in no way be construed as limiting the scope of the invention, as defined by the appended claims. Illustrated below is a sample study protocol and the identification of biomarkers that have positive or negative correlations with coronary artery disease. While the sample protocol has been developed with human blood plasma samples, the same general experimental set-up may be used for other suitable biological samples to detect biomarkers. All documents mentioned herein are fully incorporated by reference.

### EXAMPLE 1

Patients and control subjects are from the “Duke Databank for Cardiovascular Disease and the Duke Cardiac Catheterization Laboratory” of the Duke Clinical Research Institute (DCRI). After the subjects provide informed consent, additional clinical data is collected to supplement the clinical database. Patients with coronary artery disease are recruited at the time of their procedure while in the cardiac catheterization laboratory and the control population are recruited both from the cardiac catheterization laboratory and retrospectively within two years of cardiac catheterization.

Populations are defined in order to minimize differences in plasma proteins unrelated to the presence or absence of coronary artery disease. Three different cohorts of subjects and controls are enrolled:

(i) “pooled males” are matched for age and ethnic group. Major diseases known to be associated With differences in proteins such as diabetes mellitus and inflammatory conditions such as recent acute myocardial infarction or active malignancy are excluded.

(ii) “other males” do not need to comply with the matching criteria. It is the intention to included a relatively young population of patients with coronary artery disease in order to enrich the sample for factors related to coronary disease that would be expected to more prominent in a younger population.

(iii) “females” are enrolled as the third cohort to be able to define characteristics of the overall population and to assess gene polymorphisms of interest in the female population.

Inclusion criteria for the coronary disease patient population are: age between 40 and 65 and coronary artery stenosis of >50% in at least one major coronary artery. Exclusion criteria are acute myocardial infarction within one month, diabetes mellitus, uncontrolled hypertension (systolic blood pressure>180 mmHg or diastolic blood pressure>100 mmHg), and/or with end-organ damage, renal insufficiency (serum creatinine>2.0 mg/dL and/or BUN>40 mg/dL), active malignancy, significant valvular heart disease, NYHA Class III or IV heart failure, cigarette smoking>2 packs per day, total cholesterol>300 mg/dL or triglyceride>400 mg/dL, any other disease or condition expected to cause major alteration in plasma protein composition, anemia (hemoglobin <12.5 g/dL for females or <13.5 g/dL for males), and hypotension (systolic blood -pressure <90 mmHg and diastolic blood pressure<50 mmHg).

Inclusion criteria for the “control” population are: age between 40 and 65 for pooled males cohort only, no coronary artery stenosis of >25% on cardiac catheterization within two years, and normal left ventricular ejection fraction and normal regional wall motion. Exclusion criteria are typical signals of angina, or any evidence of myocardial ischemia on stress testing, myocardial infarction or unstable angina, any history of peripheral arterial or cerebrovascular disease including claudicatio, stroke, transient ischemic attack, or significant vascular stenosis on noninvasive imaging or angiography, diabetes, uncontrolled hypertension (systolic blood pressure>180 mmHg or diastolic blood pressure>100 mmHg), and/or with end-organ damage, renal insufficiency (serum creatinine>2.0 mg/dL and/or BUN>40 mg/dL), active malignancy, significant valvular heart disease, NYHA Class III or IV heart failure, cigarette smoking>2 packs per day, total cholesterol>300 mg/dL or triglyceride>400 mg/dL, any symptomatic heart failure, significant valvular disease, any other disease or condition expected to cause major alteration in plasma protein composition, anemia (hemoglobin <12.5 g/dL for females or <13.5 g/dL for males), and hypotension (systolic blood pressure<90 mmHg and diastolic blood pressure<50 mmHg).

Assessment of Clinical Parameters

Demographic information of the subjects include age at time of study (years), age at last catheterization (years), gender, race, smoking behaviour, systolic and diastolic blood pressure (mmHg), and severity of coronary artery disease (CAD-Index). Assessment of medical history includes angina, diabetes mellitus including end organ damage, hypertension, myocardial infarction, PCI, CABG, peripheral vascular disease, cerebro-vascular disease, congestive heart failure, severity of congestive heart failure, and renal insufficiency.

Regarding coronary disease following information is collected: LV ejection fraction (%), number of significantly obstructed vessels, maximal percent stenosis of the left main coronary system, maximal percent stenosis of the left anterior descending artery, maximal percent stenois of the right artery, maximal percent stenosis of the left circumflex artery system, mitral insufficiency grade, valvular disease, aortic stenosis severity disease, follow-up MI, PCI, and CABG, indication for catheterization diagnosis, if different.

Assessment of medication includes ACE inhibitors, amiodarone, angiotensin receptor blocker (ARB), aspirin products, beta-blocker, calcium blocker, central acting agents, digoxin, diuretics, dofetalid, fibrate, hormone replacement therapy, niacin compound, nitrate, other antiplatele, sotalol, statin, type 1 agents, vasodilatators, and warfarin. Clinical laboratory parameters assessed at Duke laboratory included total cholesterol, triglycerides, LDL, HDL, HbA1C, Hct, creatinine, PT, PTT, WBC, phosphorus, sodium, potassium, chloride, calcium, and carbone dioxide.

Clinical laboratory parameters include sodium, potassium, chloremia, calcium, magnesium, phosphorus, BUN, creatinine, protein total, CK total, CK-MB, LDH, γ-GT, ASAT, ALAT, alkaline phosphatase, bilirubin total, bilirubin conjugated, C-reactive protein, cholesterol, triglycerides, HDL, albumin, osmolarity, LDL calc, Ca calc, troponin l, TSH, Homocysteine, HIV1-2, HCV3, HBsAG, and HBclg.

Severity of coronary artery disease (CAD-index) is scored according to following cross-reference table:

TABLE 1
Coding of the CAD-Index, vd stands for significantly obstructed vessels

| CAD-Index | Criteria |
|---|---|
| 0 | No CAD >=50% |
| 19 | 1 vd (50-74%) |
| 23 | >1 vd (50-74%) |
| 23 | 1 vd (>=75%) |
| 32 | 1 vd severe (>=95%) |
| 37 | 2 vd |
| 42 | 2 vd, 2 severe |
| 48 | 1 vd, severe proximal LAD |
| 48 | 2 vd, severe LAD |
| 56 | 2 vd, severe proximal LAD |
| 56 | 3 vd |
| 63 | 3 vd, >=1 severe |
| 67 | 3 vd, proximal LAD disease |
| 74 | 3 vd, severe proximal LAD |
| 82 | Left main (75%) |
| 100 | Left main severe |

Blood Sampling

One aliquot of 100 mL of blood is collected from male disease ,patients, and 200 ml (2×100 mL aliquots) of blood is collected from male control subjects, in 20 mL tubes containing citrate, phosphate and dextrose. Females have 2×10 mL tubes of blood drawn. The fall in temperature to ambient is accelerated by placing the tubes in a water bath at 25° C., from the beginning of the collection process. After the end of the collection, the tubes are cooled, allowing the temperature progressively lowered to 20 - 22° C. in one hour. White blood cell and platelet reduction (less than 1×10⁶/unit) is systematically performed by filtration at room temperature, by passage of the blood on standard -gravitation filters. The bags are centrifuged in a standard fashion.

Plasma is separated from red cells using a press and collected in separate bags. Protease inhibitors are added. The tubes are frozen to −70° C., and stored prior to bulk shipment on dry ice to GeneProt, Geneva, Switzerland.

Microarray Analysis

The blood samples (2.5 mL) are collected directly into PAXgene™ Blood RNA tubes containing a progrietary blends of reagents that bring about immediate stabilization of RNA (PreAnalytiX, Qiagen). The RNA is then isolated using silica-gel-membrane technology supplied in the PAXgene Blood RNA Kit. The resulting RNA accurately represents the expression profile in vivo and is suitable for use in a range of downstream applications. The RNA isolation begins with a centrifugation step to pellet nucleic acids in the PAXgene Blood RNA Tube. The pellet is washed, and Proteinase K is added to digest proteins. Alcohol is added to adjust binding conditions, and the sample is applied to a PAXgene RNA spin column. During a brief centrifugation, RNA is selectively bound to the PAXgene silica-gel membrane as contaminants pass through. Following washing steps, RNA is eluted in an optimized buffer. Total RNA is quantified by the absorbance at λ=260 nm (A260 nm) and the purity is estimated by the ratio A260 nm/A280 nm. Integrity of the RNA molecules is confirmed by non-denaturing agarose gel electrophoresis. RNA was stored at approximately −80° C. until analysis.

DNA Micro-Array Experiment

All GeneChip experiments are conducted in the Genomics Factory EU, as recommended by the manufacturer of the GeneChip system (Affymetrix, Santa Clara, Calif.; Expression analysis technical manual: put into references). Genome U133A expression probe array set (Affymetrix, Inc., San Diego, Calif., USA) is used.

GeneChip Experiment

Double stranded cDNA is synthesized with a starting amount of approximately 1 to 10 μg full-length total RNA using the Superscript Choice System (Invitrogen Life Technologies) in the presence of a T7-(dT) 24 DNA oligonucleotide primer. Following synthesis, the cDNA is cDNA purified on an affinity resin (QiaQuick, Qiagen). The purified cDNA is then transcribed in vitro using the BioArray® High Yield RNA Transcript Labeling Kit (ENZO) in the presence of biotinylated ribonucleotides form biotin labeled CRNA. The labeled cRNA is then purified on an affinity resin (RNeasy, Qiagen), quantified and fragmented. An amount of exactly 10 μg labeled cRNA is hybridized for approximately 16 hours at 45° C. to an expression probe array. The array is then washed and stained twice with streptavidin-phycoerythrin (Molecular Probes) using the GeneChip Fluidics Workstation 400 (Affymetrix). The array is then scanned twice using a confocal laser scanner (GeneArray Scanner, Agilent) resulting in one scanned image, “.dat-file”.

Clinical Data

Clinical data are entered into a Clintrial database by DCRI, and data verification is done by double data entry. After data entry completion and lock of the database data are sent to the sponsor in an encrypted SAS format. At the sponsors site, clinical data are loaded into a MSAccess database. Descriptive statistical parameters are calculated for each of the six sub-populations; i.e. pooled male cases and controls, other male cases and controls, and female cases and controls. Descriptive statistics include sample size, frequencies, arithmetic mean, standard deviation, median, minimum and maximum values. Individual clinical data from in total 241 subjects: 121 diseased subjects and 120 controls. The pooled males cohort contain 53 diseased subjects and 53 controls, the other males cohort contain 44 diseased subjects and 38 controls, and the females cohort contain 24 diseased subjects and 29 controls.

Gene Expression Data

This resulting “.dat-file” is processed using the MAS5 program (Affymetrix) into a “.cel-file”. The “.cel file” is captured and loaded into the Affymetrix GeneChip Laboratory Information Management System (LIMS). The LIMS database is connected to a UNIX Sun Solaris server through a network filing system that allows for the average intensities for all probes cells (CEL file) to be downloaded into an Oracle database (NPGN). Raw data is converted to expression levels using a “target intensity” of 150. The numerical values displayed are weighted averages of the signal intensities of the probe-pairs comprised in a probe-set for a given transcript sequence (AvgDiff value). Individual gene expression data are obtained from 222 out of 241 patients.

PLS by Partial Least Square

PLS stands for Projections to Latent Structures by means of partial least squares. PLS finds the linear or polynomial relationship between a matrix Y containing the dependent response variables and a matrix X containing the predictor variables. PLS modeling consists of simultaneous projections of both the X and Y spaces on low dimensional hyper planes. The coordinates of the points on these hyper planes constitute the elements of the matrices T and U which are referred to as t-scores and u-scores in the graphical representation of the PLS results below. The PLS analysis has the objectives to well approximate the X and Y spaces and to maximize the correlation between X and Y; i.e. very similar objectives as the canonical correlation analysis. Regarding the X (predictors) and Y (dependent variables) matrices, the objective of orthogonal signal correction—OSC—is to remove all information from X that is unrelated (orthogonal) to Y which does not contribute information of interest. SIMCA-P Version 10.0 (Umetrics, Sweden) was used for predictive modeling by PLS.

Data Integration

Clinical data, gene expression data are linked by a bar-code, that uniquely identified a subject across all data sets

Study Population

The study population consisted of three cohorts. In the first cohort—referred to as “pooled males”—were 53 male controls and 53 male cases. These individuals were matched according to their age and race. The second cohort termed “other males” contained 38 male controls and 44 male cases and the third cohort termed “females” was made of 29 female controls and 24 cases. In total, 241 individuals participated in this clinical investigation with 120 controls and 121 cases. 188 out of 241 individuals were male and 53 female.

Demographic Information

The average age of the study population was between 51.1 and 57.6 years with the smallest difference between cases and controls in the “pooled males” cohort. Body weight of males was in the range of 93.9-100.6 kg and 83.9-89.5 kg for females. In general, controls had a higher body weight than the cases. Frequency of smoking was 29/53 in the pooled male cases and 26/53 in pooled male controls, 34/44 in other male cases and 20/38 in other male controls, and 13/24 female cases and 9/29 female controls indicating that there were more smokers in the cases compared to controls. Systolic and diastolic blood pressure of the individuals was on average in the normal range. However, there is a tendency for higher blood pressure in the controls compared to cases. By far most of the study population belonged to the Caucasian race, followed by Afro-Americans and a few Asians and Hispanics.

Medical History

Angina pectoris was the most prominent clinical event in each of the cohorts with no obvious difference between cases and controls. However, history of hypertension was more reported by the male cases compared to controls. There was no difference in the female cohort. Myocardial infarction was significantly higher in the cases of all cohort as well as coronary artery by-pass graft (CABG) and peripheral coronary intervention (PCI). A few of the cases reported coronary heart failure (CHF), but none of the controls. Very few of the cases suffered from peripheral or cerebral vascular disease, but none of the control. Diabetes mellitus was not reported and renal insufficiency in two subjects only.

Medication

Most of the patients took at least one medication (NSAIDs) is the class of medication that is taken by most of the subjects and there is no difference between cases and controls or between the three cohorts. Blood pressure lowering agents such as beta-blockers, ACE-inhibitors, calcium channel blockers, diuretics, and angiotensin receptor blockers were in general more frequently taken by the cases compared to the controls. The same holds true for statins. Less frequent medication included amioderone, fibrate, digoxin, nitrate, niacin, and anticoagulants which were taken more often by cases than controls. Some of the females took hormone replacement and only a few patients took centrally acting medication.

Severity of Coronary Artery Disease—CAD-index

Amongst the cases, however, there was a wide distribution; 52% of the cases had CAD-Indices below 42; 79% of the cases had CAD-Indices below 56; and the remaining 21% of cases had CAD-Indices between 63 and 100%. Most of the controls are found in the CAD-Index range between 15 and 22 but all CAD-values below 23 were by default set to zero since CAD-Indices of zero have a tremendously high leverage effect on all types of regression analyses.

TABLE 2
Study population

| Cohort | Sample Size |
|---|---|
| Pooled Male | 53 controls |
| | 53 cases |
| Other Males | 38 controls |
| | 44 cases |
| Females | 29 controls |
| | 24 cases |
| Total | 241 |
| | 120 controls, 121 cases |
| | 188 males, 53 females |

TABLE 3
Mean demographic data

| | Pooled Males | | Other Males | | Females | |
|---|---|---|---|---|---|---|
| | Cases | Controls | Cases | Controls | Cases | Controls |
| Age (yrs) | 52.7 | 52.3 | 57.6 | 51.1 | 54.2 | 51.9 |
| Weight (kg) | 93.9 | 96.8 | 94.3 | 100.6 | 83.9 | 89.5 |
| Smoking | 29 | 26 | 34 | 20 | 13 | 9 |
| SBP (mmHg) | 134 | 139 | 142 | 145 | 138 | 145 |
| DBP (mmHg) | 76 | 80 | 82 | 82 | 70 | 77 |
| Race | | | | | | |
| Caucasian | 50 | 50 | 39 | 23 | 18 | 21 |
| Afro-american | 3 | 3 | 4 | 13 | 4 | 5 |
| Native | | | 1 | | | 2 |
| american | | | | | | |
| Asian | | | | 1 | 1 | |
| Hispanic | | | | 1 | | 1 |
| Others | | | | | 1 | |

TABLE 4
Summary of Medical History (frequencies)

| | | | Other Males | | Females | |
|---|---|---|---|---|---|---|
| | Pooled Males | | | Con- | | Con- |
| | Cases | Controls | Cases | trols | Cases | trols |
| Angina Pectoris | 48 | 47 | 42 | 35 | 23 | 25 |
| Hypertension | 27 | 16 | 27 | 15 | 12 | 13 |
| Myocardial.Infarction | 29 | 0 | 21 | 2 | 10 | 1 |
| CABG | 15 | 0 | 15 | 0 | 7 | 0 |
| PCI | 15 | 0 | 13 | 0 | 4 | 0 |
| CHF | 6 | 0 | 4 | 0 | 2 | 0 |
| Peripheral Vascular | 2 | 0 | 5 | 0 | 3 | 0 |
| Disease | | | | | | |
| CerebralVascular | 1 | 0 | 4 | 0 | 3 | 0 |
| Disease | | | | | | |
| Diabetes mellitus | 0 | 0 | 0 | 0 | 0 | 0 |
| Renal Insufficiency | 0 | 1 | 1 | 0 | 0 | 0 |

TABLE 5
Summary of Medication (frequencies)

| | | | | | Females | |
|---|---|---|---|---|---|---|
| | Males Pooled | | Other Males | | | Con- |
| | Cases | Controls | Cases | Controls | Cases | trols |
| NSAIDs | 47 | 18 | 42 | 10 | 21 | 6 |
| Beta-Blocker | 43 | 13 | 37 | 6 | 20 | 6 |
| ACE Inhibitor | 40 | 5 | 26 | 7 | 13 | 4 |
| Calcium Blocker | 10 | 4 | 9 | 5 | 4 | 3 |
| Diuretic | 8 | 6 | 12 | 5 | 7 | 7 |
| Angiotensin RB | 0 | 3 | 5 | 2 | 5 | 0 |
| Statins | 38 | 10 | 29 | 4 | 15 | 5 |
| Amiodarone | 1 | 0 | 0 | 0 | 0 | 0 |
| Fibrate | 4 | 2 | 8 | 0 | 1 | 0 |
| Digoxin | 2 | 0 | 0 | 0 | 1 | 0 |
| Nitrate | 12 | 0 | 6 | 1 | 5 | 0 |
| Niancin | 3 | 0 | 1 | 0 | 1 | 0 |
| Antiplatelet | 8 | 1 | 1 | 0 | 4 | 1 |
| Hormon Replacemt | 0 | 0 | 1 | 0 | 9 | 5 |
| Central Acting | 1 | 0 | 1 | 1 | 0 | 0 |

Correlation of Gene Expression and Laboratory Values Incl. CAD-Index

A univariate correlation analysis by parametric and non-parametric methods was applied to all laboratory values and all genes. The CAD-index was included in the set of laboratory parameters. The gene filtering of all subsequent univariate and multivariate methods was based on this correlation analysis; i.e. only genes were included in the analysis that exhibited an absolute correlation coefficient with CAD Index greater an arbitrary level, abs(rho)>0.2 for the partial least squares analysis.

Partial Least Squares (PLS) projection to latent structures is an alternative modeling approach to modeling multivariate response data. Due to its optimized algorithm it is able to cope with short and fat matrices; i.e. the number of variables can be much greater than the number of observations.

160 genes correlated with CAD-Index with absolute correlation coefficients of rho>0.2 (Table 6); these genes were included in the PLS analysis. Before performing the LPS the gene expression data were subjected to Orthogonal Signal Correction (OSC) with CAD-Index as the only response variable.

TABLE 6
Means and p values of each of the 160 genes

| | | | | | | | worst/best | |
|---|---|---|---|---|---|---|---|---|
| | | | | | | | (n = 15 | |
| | | | | all | | | per group) | |
| | | | | (n = 221) | | | SigLog | t-test |
| Accession# | Unigene# | Probeset | Gene | VIP | Corr | Rho | Ratio | p-value |
| NM_000146.2 | Hs.433670 | 212788_x_at | FTL | 10.63 | 0.24 | 0.24 | 1.18 | 0.008 |
| NM_012181.2 | Hs.173464 | 40850_at | FKBP8 | 3.69 | 0.22 | 0.25 | 1.39 | 0.0795 |
| NM_006082.1 | Hs.446608 | 212639_x_at | K-ALPHA-1 | 2.92 | 0.22 | 0.28 | 1.22 | 0.0196 |
| NM_020376.2 | Hs.118463 | 39854_r_at | TTS-2.2 | 2.49 | 0.25 | 0.24 | 1.45 | 0.0192 |
| NM_025241.1 | Hs.435255 | 220757_s_at | UBXD1 | 2.43 | 0.25 | 0.21 | 1.51 | 0.0155 |
| NM_016496.3 | Hs.331308 | 210075_at | LOC51257 | 1.2 | 0.22 | 0.24 | 1.44 | 0.0023 |
| NM_014216.3 | Hs.408429 | 210740_s_at | ITPK1 | 0.99 | 0.29 | 0.3 | 1.25 | 0.0021 |
| NM_032409.1 | Hs.439600 | 209018_s_at | PINK1 | 0.88 | 0.22 | 0.22 | 1.47 | 0.001 |
| NM_007219.2 | Hs.30524 | 210706_s_at | RNF24 | 0.82 | 0.22 | 0.28 | 1.12 | 0.0946 |
| NM_006690.2 | Hs.212581 | 78047_s_at | MMP24 | 0.82 | 0.23 | 0.2 | 1.29 | 0.0125 |
| NM_006949.1 | Hs.379204 | 209367_at | STXBP2 | 0.78 | 0.22 | 0.25 | 1.19 | 0.0375 |
| NM_002957.3 | Hs.20084 | 202426_s_at | RXRA | 0.74 | 0.25 | 0.24 | 1.34 | 0.012 |
| NM_174930.2 | Hs.397073 | 179_at | PMS2L5 | 0.68 | 0.26 | 0.24 | 1.28 | 0.0243 |
| NM_022107.1 | Hs.288316 | 214847_s_at | GPSM3 | 0.68 | 0.25 | 0.27 | 1.28 | 0.0052 |
| NM_004364.2 | Hs.76171 | 204039_at | CEBPA | 0.57 | 0.3 | 0.35 | 1.3 | 0.0279 |
| | Hs.323712 | 48612_at | N4BP1 | 0.56 | 0.21 | 0.21 | 1.19 | 0.0149 |
| NM_012295.2 | Hs.435798 | 202624_s_at | CABIN1 | 0.51 | 0.22 | 0.21 | 1.28 | 0.009 |
| NM_001119.3, | Hs.183706 | 214726_x_at | ADD1 | 0.48 | 0.23 | 0.21 | 1.26 | 0.0208 |
| NM_001119.3, | | | | | | | | |
| NM_176801.1, | | | | | | | | |
| NM_014190.2 | | | | | | | | |
| NM_021933.1 | Hs.8595 | 48659_at | FLJ12438 | 0.46 | 0.2 | 0.22 | 1.15 | 0.076 |
| NM_003023.2 | Hs.167679 | 209370_s_at | SH3BP2 | 0.43 | 0.21 | 0.23 | 1.21 | 0.0156 |
| NM_001250.3, | Hs.504816 | 35150_at | TNFRSF5 | 0.43 | 0.23 | 0.22 | 1.19 | 0.0592 |
| NM_001250.3 | | | | | | | | |
| NM_015444.1 | Hs.35861 | 213338_at | RIS1 | 0.42 | 0.22 | 0.28 | 3.13 | 0.0007 |
| NM_002659.1 | Hs.179657 | 211924_s_at | PLAUR | 0.4 | 0.22 | 0.2 | 1.36 | 0.0052 |
| NM_002308.2, | Hs.81337 | 203236_s_at | LGALS9 | 0.39 | 0.2 | 0.25 | 1.34 | 0.0087 |
| NM_002308.2 | | | | | | | | |
| NM_002319.2 | Hs.125742 | 204692_at | LRRN4 | 0.38 | 0.23 | 0.26 | 1.2 | 0.0523 |
| NM_033200.1 | Hs.150540 | 31837_at | BC002942 | 0.38 | 0.22 | 0.28 | 1.15 | 0.0994 |
| NM_001487.1 | Hs.94672 | 202592_at | GCN5L1 | 0.37 | 0.21 | 0.23 | 1.11 | 0.0955 |
| NM_006122.1, | Hs.116459 | 202032_s_at | MAN2A2 | 0.36 | 0.24 | 0.28 | 1.41 | 0.0001 |
| NM_006122.1 | | | | | | | | |
| NM_005474.3, | Hs.9028 | 202455_at | HDAC5 | 0.35 | 0.22 | 0.28 | 1.09 | 0.2789 |
| NM_005474.3 | | | | | | | | |
| NM_004317.1 | Hs.165439 | 202024_at | ASNA1 | 0.3 | 0.22 | 0.21 | 1.24 | 0.0049 |
| NM_000201.1 | Hs.386467 | 202637_s_at | ICAM1 | 0.3 | 0.2 | 0.21 | 1.26 | 0.02 |
| NM_014223.2 | Hs.285133 | 202215_s_at | NFYC | 0.26 | 0.23 | 0.3 | 1.1 | 0.1915 |
| NM_002504.3, | Hs.413074 | 202585_s_at | NFX1 | 0.19 | 0.28 | 0.26 | 1.29 | 0.0351 |
| NM_002504.3, | | | | | | | | |
| NM_147133.1 | | | | | | | | |
| NM_000430.2 | Hs.77318 | 200815_s_at | PAFAH1B1 | 0.17 | 0.23 | 0.27 | 1.29 | 0.009 |
| NM_024408.2 | Hs.502564 | 202445_s_at | NOTCH2 | 0.16 | 0.24 | 0.2 | 1.68 | 0.0133 |
| NM_003714.1 | Hs.155223 | 203439_s_at | STC2 | 0.14 | 0.2 | 0.24 | 1.31 | 0.1213 |
| NM_004229.2 | Hs.407604 | 202612_s_at | CRSP2 | 0.13 | 0.26 | 0.24 | 2.34 | 0.0118 |
| NM_006379.2 | Hs.171921 | 203788_s_at | SEMA3C | 0.11 | 0.21 | 0.28 | 1.17 | 0.244 |
| NM_003107.2 | Hs.357901 | 201416_at | SOX4 | 0.1 | 0.21 | 0.2 | 1.74 | 0.018 |
| NM_003463.2 | Hs.227777 | 200730_s_at | PTP4A1 | 0.09 | 0.23 | 0.27 | 1.32 | 0.0552 |
| NM_021016.2 | Hs.438687 | 203399_x_at | PSG3 | 0.08 | 0.22 | 0.22 | 1.38 | 0.3666 |
| NM_005334.1 | Hs.83634 | 202473_x_at | HCFC1 | 0.03 | 0.22 | 0.23 | 1.24 | 0.1219 |
| NM_006411.2, | Hs.332138 | 32836_at | AGPAT1 | 0.35 | 0.25 | 0.29 | 1.15 | 0.0444 |
| NM_006411.2 | | | | | | | | |
| NM_000086.1 | Hs.194660 | 209275_s_at | CLN3 | 0.33 | 0.2 | 0.27 | 1.12 | 0.2928 |
| | | 222302_at | | 0.32 | 0.23 | 0.22 | 1.53 | 0.0319 |
| NM_004140.2 | Hs.95659 | 206123_at | LLGL1 | 0.3 | 0.23 | 0.22 | 1.85 | 0.0032 |
| NM_007283.4 | Hs.409826 | 211026_s_at | MGLL | 0.3 | 0.23 | 0.27 | 1.28 | 0.0076 |
| NM_001666.2 | Hs.3109 | 204425_at | ARHGAP4 | 0.29 | 0.21 | 0.23 | 1.09 | 0.4942 |
| NM_014001.2, | Hs.87726 | 211815_s_at | GGA3 | 0.28 | 0.26 | 0.26 | 1.26 | 0.0536 |
| NM_014001.2 | | | | | | | | |
| NM_018310.2 | Hs.17270 | 218955_at | BRF2 | 0.28 | 0.23 | 0.23 | 1.38 | 0.0001 |
| NM_016531.2 | Hs.145754 | 219657_s_at | KLF3 | 0.28 | 0.25 | 0.21 | 1.38 | 0 |
| NM_018986.2 | Hs.61053 | 219256_s_at | SH3TC1 | 0.27 | 0.29 | 0.28 | 1.31 | 0.006 |
| NM_024009.1 | Hs.488738 | 215243_s_at | GJB3 | 0.26 | 0.2 | 0.24 | 1.25 | 0.0536 |
| NM_000508.2, | Hs.351593 | 205650_s_at | FGA | 0.25 | 0.21 | 0.26 | 1.64 | 0.024 |
| NM_000508.2 | | | | | | | | |
| NM_000964.1 | Hs.361071 | 211605_s_at | RARA | 0.25 | 0.22 | 0.26 | 1.43 | 0.0187 |
| NM_003805.2 | Hs.155566 | 209833_at | CRADD | 0.24 | 0.26 | 0.25 | 1.42 | 0.0038 |
| NM_012241.2, | Hs.282331 | 221010_s_at | SIRT5 | 0.23 | 0.22 | 0.21 | 1.78 | 0.0258 |
| NM_012241.2 | | | | | | | | |
| NM_017883.3 | Hs.12142 | 222138_s_at | WDR13 | 0.23 | 0.21 | 0.24 | 1.32 | 0.0224 |
| NM_001625.2, | Hs.294008 | 212174_at | AK2 | 0.22 | 0.27 | 0.28 | 1.1 | 0.3327 |
| NM_001625.2, | | | | | | | | |
| NM_172199.1 | | | | | | | | |
| NM_000136.1 | Hs.253236 | 205189_s_at | FANCC | 0.21 | 0.21 | 0.23 | 1.39 | 0.0115 |
| NM_005955.1 | Hs.211581 | 205323_s_at | MTF1 | 0.21 | 0.21 | 0.25 | 1.09 | 0.2811 |
| NM_006693.1 | Hs.434994 | 206688_s_at | CPSF4 | 0.21 | 0.21 | 0.22 | 1.12 | 0.1921 |
| NM_004245.1, | Hs.129719 | 207911_s_at | TGM5 | 0.21 | 0.22 | 0.24 | 1.52 | 0.1156 |
| NM_004245.1 | | | | | | | | |
| NM_014922.3, | Hs.104305 | 211822_s_at | NALP1 | 0.21 | 0.21 | 0.26 | 1.2 | 0.052 |
| NM_014922.3, | | | | | | | | |
| NM_021621, | | | | | | | | |
| NM_021730, | | | | | | | | |
| NM_033004.2, | | | | | | | | |
| NM_033005, | | | | | | | | |
| NM_033006.2 | | | | | | | | |
| XM_093895.6 | Hs.411317 | 212960_at | KIAA0882 | 0.21 | 0.24 | 0.22 | 1.58 | 0.0032 |
| NM_002652.1 | Hs.99949 | 206509_at | PIP | 0.2 | 0.27 | 0.23 | 2.23 | 0.0016 |
| NM_002049.2 | Hs.765 | 210446_at | GATA1 | 0.2 | 0.23 | 0.25 | 1.69 | 0.0517 |
| NM_005203.3, | Hs.211933 | 211809_x_at | COL13A1 | 0.2 | 0.24 | 0.25 | 2.23 | 0.0017 |
| NM_005203.3, | | | | | | | | |
| NM_080804.2, | | | | | | | | |
| NM_080813.2, | | | | | | | | |
| NM_080812.2, | | | | | | | | |
| NM_080811.2, | | | | | | | | |
| NM_080810.2, | | | | | | | | |
| NM_080809.2, | | | | | | | | |
| NM_080808.2, | | | | | | | | |
| NM_080807.2, | | | | | | | | |
| NM_080806.2, | | | | | | | | |
| NM_080805.2, | | | | | | | | |
| NM_080815.2, | | | | | | | | |
| NM_080814.2, | | | | | | | | |
| NM_080803.2, | | | | | | | | |
| NM_080798.2, | | | | | | | | |
| NM_080800.2, | | | | | | | | |
| NM_080801.2, | | | | | | | | |
| NM_080802.2 | | | | | | | | |
| NM_006244.2 | Hs.75199 | 635_s_at | PPP2R5B | 0.19 | 0.21 | 0.2 | 1.45 | 0.0993 |
| NM_002588.2, | Hs.283794 | 205717_x_at | PCDHGC3 | 0.18 | 0.24 | 0.2 | 1.54 | 0.1409 |
| NM_002588.2, | | | | | | | | |
| NM_032402.1 | | | | | | | | |
| NM_024608.1 | Hs.512732 | 219396_s_at | NEIL1 | 0.18 | 0.23 | 0.31 | 1.22 | 0.1956 |
| NM_015478.4, | Hs.300863 | 206822_s_at | L3MBTL | 0.17 | 0.33 | 0.32 | 2.44 | 0.005 |
| NM_015478.4 | | | | | | | | |
| NM_004711.3, | Hs.414343 | 213854_at | SYNGR1 | 0.17 | 0.23 | 0.22 | 1.94 | 0.003 |
| NM_004711.3, | | | | | | | | |
| NM_145731.2 | | | | | | | | |
| NM_002586.3 | Hs.93728 | 211097_s_at | PBX2 | 0.16 | 0.2 | 0.25 | 1.24 | 0.3167 |
| NM_025188.1 | Hs.301526 | 219923_at | TRIM45 | 0.16 | 0.26 | 0.23 | 1.49 | 0.0017 |
| NM_004055.3 | Hs.248153 | 205166_at | CAPN5 | 0.15 | 0.21 | 0.25 | 1.49 | 0.0165 |
| NM_002186.2, | Hs.406228 | 208164_s_at | IL9R | 0.15 | 0.24 | 0.24 | 2.67 | 0.0017 |
| NM_002186.2 | | | | | | | | |
| NM_003547.2 | Hs.519634 | 208551_at | HIST1H4G | 0.15 | 0.24 | 0.27 | 1.45 | 0.2141 |
| NM_004137.2 | Hs.93841 | 209948_at | KCNMB1 | 0.15 | 0.2 | 0.22 | 1.26 | 0.0723 |
| NM_032336.1 | Hs.333166 | 211767_at | MGC14799 | 0.15 | 0.3 | 0.27 | 1.53 | 0.1624 |
| NM_015277.2 | Hs.249798 | 212445_s_at | NEDD4L | 0.15 | 0.21 | 0.22 | 1.32 | 0.0629 |
| NM_002735.1 | Hs.478057 | 212559_at | PRKAR1B | 0.15 | 0.25 | 0.25 | 1.74 | 0.0163 |
| NM_001171.2 | Hs.442182 | 214033_at | ABCC6 | 0.15 | 0.23 | 0.26 | 1.52 | 0.0505 |
| | | 215906_at | | 0.15 | 0.21 | 0.28 | 2.13 | 0.0052 |
| NM_006093.2 | Hs.251386 | 220811_at | PRG3 | 0.15 | 0.21 | 0.24 | 2.29 | 0.0121 |
| NM_001218.2, | Hs.279916 | 203963_at | CA12 | 0.14 | 0.22 | 0.26 | 2.95 | 0 |
| NM_001218.2 | | | | | | | | |
| NM_000228.1 | Hs.436983 | 209270_at | LAMB3 | 0.14 | 0.26 | 0.25 | 2.58 | 0.0007 |
| NM_173834.2 | Hs.82719 | 212340_at | MGC21416 | 0.14 | 0.22 | 0.27 | 1.38 | 0.0379 |
| NM_006034.2 | Hs.385634 | 214667_s_at | TP53I11 | 0.14 | 0.27 | 0.31 | 2.01 | 0.0094 |
| | | 215971_at | | 0.14 | 0.22 | 0.21 | 1.53 | 0.1513 |
| NM_004476.1 | Hs.1915 | 217487_x_at | FOLH1 | 0.14 | 0.22 | 0.22 | 1.55 | 0.0358 |
| NM_017715.1, | Hs.435302 | 219605_at | ZNF3 | 0.14 | 0.26 | 0.22 | 1.88 | 0.0096 |
| NM_017715.1 | | | | | | | | |
| NM_024735.2 | Hs.371923 | 219784_at | MGC15419 | 0.14 | 0.22 | 0.26 | 1.37 | 0.1724 |
| NM_025117.1 | Hs.288727 | 220915_s_at | FLJ11871 | 0.14 | 0.27 | 0.24 | 2.34 | 0.0155 |
| NM_006365.1 | Hs.380027 | 222301_at | CROC4 | 0.14 | 0.2 | 0.22 | 1.27 | 0.1667 |
| NM_003914.2 | Hs.417050 | 205899_at | CCNA1 | 0.13 | 0.21 | 0.2 | 1.16 | 0.5073 |
| NM_002640.3, | Hs.368077 | 206034_at | SERPINB8 | 0.13 | 0.21 | 0.23 | 1.44 | 0.0271 |
| NM_002640.3 | | | | | | | | |
| NM_005622.2, | Hs.512678 | 210377_at | SAH | 0.13 | 0.26 | 0.3 | 1.84 | 0.0035 |
| NM_005622.2 | | | | | | | | |
| NM_007033.2 | Hs.40500 | 213114_at | RER1 | 0.13 | 0.22 | 0.26 | 1.8 | 0.0476 |
| NM_015896.2 | Hs.167380 | 216663_s_at | ZMYND10 | 0.13 | 0.22 | 0.25 | 2.19 | 0.0109 |
| NM_024893.1 | Hs.233634 | 219310_at | C20orf39 | 0.13 | 0.23 | 0.21 | 2.16 | 0.0048 |
| NM_016931.2 | Hs.371036 | 219773_at | NOX4 | 0.13 | 0.26 | 0.23 | 2.01 | 0.1031 |
| NM_003299.1 | Hs.192374 | 216450_x_at | TRA1 | 0.12 | 0.2 | 0.2 | 1.22 | 0.4391 |
| | | 217212_s_at | | 0.12 | 0.21 | 0.2 | 1.39 | 0.3622 |
| NM_016158.1 | Hs.104671 | 220752_at | LOC51145 | 0.12 | 0.27 | 0.23 | 1.6 | 0.1176 |
| NM_006862.2 | Hs.144439 | 221053_s_at | TDRKH | 0.12 | 0.24 | 0.26 | 1.56 | 0.1105 |
| NM_014393.1 | Hs.511992 | 204226_at | STAU2 | 0.11 | 0.21 | 0.26 | 1.28 | 0.024 |
| NM_000240.2 | Hs.183109 | 204388_s_at | MAOA | 0.11 | 0.23 | 0.28 | 1.33 | 0.3663 |
| NM_001941.2, | Hs.41690 | 206032_at | DSC3 | 0.11 | 0.24 | 0.2 | 1.52 | 0.1093 |
| NM_001941.2 | | | | | | | | |
| | Hs.134816 | 206507_at | ZNF305 | 0.11 | 0.24 | 0.21 | 1.46 | 0.1793 |
| NM_017721.2 | Hs.269592 | 207083_s_at | FLJ20241 | 0.11 | 0.29 | 0.24 | 3.19 | 0.0002 |
| XM_372810.1 | Hs.300622 | 207290_at | PLXNA2 | 0.11 | 0.22 | 0.21 | 1.81 | 0.0418 |
| NM_001623.3, | Hs.76364 | 207823_s_at | AIF1 | 0.11 | 0.23 | 0.21 | 1.73 | 0.0957 |
| NM_001623.3, | | | | | | | | |
| NM_004847.2 | | | | | | | | |
| NM_023037.1 | Hs.390874 | 214319_at | 13CDNA73 | 0.11 | 0.25 | 0.24 | 2.38 | 0.0004 |
| | | 215763_at | | 0.11 | 0.27 | 0.26 | 1.81 | 0.1446 |
| NM_020374.2 | Hs.296198 | 218374_s_at | C12orf4 | 0.11 | 0.25 | 0.24 | 1.3 | 0.0548 |
| NM_016260.1 | Hs.278963 | 220567_at | ZNFN1A2 | 0.11 | 0.21 | 0.23 | 2.12 | 0.0201 |
| | | 221137_at | | 0.11 | 0.24 | 0.22 | 1.12 | 0.6265 |
| NM_001503.2, | Hs.512001 | 206265_s_at | GPLD1 | 0.1 | 0.29 | 0.21 | 1.53 | 0.3202 |
| NM_001503.2 | | | | | | | | |
| NM_000818.1 | Hs.231829 | 216651_s_at | GAD2 | 0.1 | 0.22 | 0.21 | 2.07 | 0.0085 |
| NM_018286.1 | Hs.173233 | 219230_at | FLJ10970 | 0.1 | 0.25 | 0.21 | 2.23 | 0.0213 |
| NM_014332.1 | Hs.86492 | 219772_s_at | SMPX | 0.1 | 0.25 | 0.2 | 2.24 | 0.0193 |
| NM_005554.2 | Hs.367762 | 209125_at | KRT6A | 0.09 | 0.24 | 0.28 | 1.74 | 0.0848 |
| NM_000268.2, | Hs.902 | 211092_s_at | NF2 | 0.09 | 0.26 | 0.23 | 1.7 | 0.0358 |
| NM_000268.2, | | | | | | | | |
| NM_016418.4, | | | | | | | | |
| NM_181825.1, | | | | | | | | |
| NM_181826.1, | | | | | | | | |
| NM_181827.1, | | | | | | | | |
| NM_181828.1, | | | | | | | | |
| NM_181829.1, | | | | | | | | |
| NM_181830.1, | | | | | | | | |
| NM_181831.1, | | | | | | | | |
| NM_181832.1, | | | | | | | | |
| NM_181833.1, | | | | | | | | |
| NM_181834.1 | | | | | | | | |
| NM_021189.2 | Hs.365689 | 213948_x_at | IGSF4B | 0.09 | 0.22 | 0.25 | 1.42 | 0.2795 |
| NM_022112.1 | Hs.160953 | 220403_s_at | P53AIP1 | 0.09 | 0.22 | 0.25 | 2.48 | 0.01 |
| NM_018423.1 | Hs.24979 | 221696_s_at | DKFZp761P1010 | 0.09 | 0.21 | 0.22 | 1.62 | 0.0547 |
| NM_005903.4 | Hs.167700 | 205187_at | MADH5 | 0.08 | 0.25 | 0.22 | 1.96 | 0.0856 |
| NM_000745.2 | Hs.1614 | 206533_at | CHRNA5 | 0.08 | 0.2 | 0.21 | 1.46 | 0.1336 |
| NM_001980.2, | Hs.99865 | 207346_at | EPIM | 0.08 | 0.23 | 0.22 | 1.71 | 0.0514 |
| NM_001980.2 | | | | | | | | |
| NM_002399.2, | Hs.362805 | 207480_s_at | MEIS2 | 0.08 | 0.2 | 0.2 | 1.2 | 0.4323 |
| NM_002399.2, | | | | | | | | |
| NM_020149.2, | | | | | | | | |
| NM_170674.2, | | | | | | | | |
| NM_170675.2, | | | | | | | | |
| NM_170676.2, | | | | | | | | |
| NM_170677.2, | | | | | | | | |
| NM_172315.1 | | | | | | | | |
| NM_004432.1 | Hs.166109 | 208427_s_at | ELAVL2 | 0.08 | 0.22 | 0.25 | 3 | 0.0028 |
| NM_006203.2 | Hs.28482 | 210837_s_at | PDE4D | 0.08 | 0.22 | 0.22 | 1.51 | 0.1544 |
| NM_001791.2, | Hs.355832 | 214230_at | CDC42 | 0.08 | 0.2 | 0.23 | 1.84 | 0.0408 |
| NM_001791.2 | | | | | | | | |
| NM_031456.2 | Hs.158313 | 215999_at | C17orf1A | 0.08 | 0.21 | 0.23 | 2.01 | 0.0104 |
| NM_018144.2 | Hs.368481 | 219499_at | SEC61A2 | 0.08 | 0.21 | 0.2 | 1.49 | 0.1369 |
| NM_000908.1 | Hs.237028 | 219789_at | NPR3 | 0.08 | 0.21 | 0.21 | 1.58 | 0.1612 |
| NM_002429.2, | Hs.154057 | 204575_s_at | MMP19 | 0.07 | 0.2 | 0.22 | 2.04 | 0.0192 |
| NM_002429.2, | | | | | | | | |
| NM_022790.1, | | | | | | | | |
| NM_022791.1 | | | | | | | | |
| NM_002849.2, | Hs.198288 | 206084_at | PTPRR | 0.07 | 0.24 | 0.23 | 1.73 | 0.1892 |
| NM_002849.2 | | | | | | | | |
| NM_000811.1 | Hs.90791 | 207182_at | GABRA6 | 0.07 | 0.21 | 0.23 | 2.12 | 0.0372 |
| NM_015594.1 | Hs.241421 | 208008_at | DKFZP434O047 | 0.07 | 0.2 | 0.25 | 1.53 | 0.1304 |
| XM_371887.1, | Hs.134792 | 212475_at | KIAA0241 | 0.07 | 0.21 | 0.28 | 1.68 | 0.0231 |
| XM_371887.1 | | | | | | | | |
| NM_015341.2 | Hs.308045 | 212949_at | BRRN1 | 0.07 | 0.25 | 0.32 | 1.37 | 0.0983 |
| NM_002026.1, | Hs.418138 | 214702_at | FN1 | 0.07 | 0.23 | 0.21 | 1.78 | 0.2234 |
| NM_002026.1 | | | | | | | | |
| NM_004993.2, | Hs.419756 | 216657_at | MJD | 0.07 | 0.2 | 0.2 | 1.41 | 0.2134 |
| NM_004993.2 | | | | | | | | |
| NM_019086.2 | Hs.333157 | 220137_at | FLJ20674 | 0.07 | 0.22 | 0.24 | 1.5 | 0.1801 |
| NM_018165.2, | Hs.173220 | 221212_x_at | PB1 | 0.07 | 0.26 | 0.25 | 3.58 | 0.0075 |
| NM_018165.2, | | | | | | | | |
| NM_018313.2, | | | | | | | | |
| NM_181041.1 | | | | | | | | |
| NM_002845.2 | Hs.154151 | 207487_at | PTPRM | 0.06 | 0.24 | 0.24 | 1.88 | 0.0884 |
| NM_000668.3 | Hs.4 | 209613_s_at | ADH1B | 0.06 | 0.21 | 0.22 | 2.33 | 0.0342 |
| NM_001797.2, | Hs.443435 | 207173_x_at | CDH11 | 0.05 | 0.2 | 0.24 | 2.43 | 0.0033 |
| NM_001797.2 | | | | | | | | |
| NM_003628.2 | Hs.277132 | 214874_at | PKP4 | 0.05 | 0.21 | 0.22 | 1.36 | 0.3519 |
| NM_018342.2 | Hs.176227 | 219750_at | FLJ11155 | 0.05 | 0.21 | 0.23 | 2.07 | 0.0622 |
| NM_000586.2 | Hs.89679 | 207849_at | IL2 | 0.04 | 0.2 | 0.22 | 1.57 | 0.2808 |
| NM_025210.1 | Hs.127689 | 207377_at | I-4 | 0.03 | 0.22 | 0.21 | 1.83 | 0.009 |
| NM_000411.3 | Hs.371350 | 207833_s_at | HLCS | 0.03 | 0.2 | 0.21 | 1.27 | 0.2197 |
| NM_018052.3 | Hs.445061 | 216501_at | FLJ10305 | 0.03 | 0.21 | 0.25 | 1.62 | 0.0438 |
| | Hs.512631 | 220787_at | PRO2533 | 0.03 | 0.21 | 0.23 | 1.99 | 0.0796 |
| | | 222308_x_at | | 0.03 | 0.22 | 0.24 | 1.32 | 0.4963 |
| NM_004789.3 | Hs.1569 | 206140_at | LHX2 | 0.02 | 0.23 | 0.21 | 1.63 | 0.0702 |
| NM_004442.4, | Hs.125124 | 211165_x_at | EPHB2 | 0.02 | 0.21 | 0.22 | 1.42 | 0.0743 |
| NM_004442.4 | | | | | | | | |

Second degree polynomial regression analysis of the resulting t-scores versus CAD-index resulted in the prediction model including 95% confidence range of the regression and the 95% prediction interval with r²=0.764 (p<0.001). The rather wide prediction interval is due to both, variability in CAD-Index assessment which is only semi-quantitative and variability in gene expression.

In order to test for stability of the model, the PLS analysis was performed separately for each of the three cohorts; i.e. “Pooled Males”, “Other Males”, and “Females”. While the controls remain quite stable in the range of −2 standard deviations, the ti-scores of the cases were located mainly in the +2 standard deviation range and increase with increasing CAD-Index. This relationship is present in each cohort with the lowest variation in the “Other Males”. Variable Importance in the Projection (VIP) of each gene for the separate PLS analyses of the three different cohorts compared to the PLS analysis including all subjects are shown in text FIG. 5. Displayed are the 24 genes with the highest VIP. The curve shows a steep decrease for the first 8 genes; thereafter, the decrease becomes rather flat and is almost linear. Apart from these eight genes all other genes contribute only marginally to projection. The VIP of the first 24 genes shows only little variation between the different cohorts pointing to a rather high stability of the prediction model.

The identification of the eight highly predictive genes that are able to predict the CAD-Index is given in Table 7.

TABLE 7
Identification of the 8 genes that contribute mostly to the prediction model
(Model 1)

| | | | | Fold |
|---|---|---|---|---|
| Probeset | Symbol | SEQ ID NO. | Gene Name | change |
| 212788_x_at | Ferritin | SEQ ID NO. 1 | ESTs, Highly similar to | 1.2 |
| | | | FRIL_HUMAN Ferritin light chain | |
| | | | (Ferritin L subunit) [H. sapiens], iron | |
| | | | ion homeostasis | |
| 40850_at | FKBP8 | SEQ ID NO. 2 | FK506 binding protein 8, 38 kDa | 1.4 |
| 212639_x_at | TUBA3 | SEQ ID NO. 3 | tubulin, alpha, ubiquitous | 1.2 |
| 39854_r_at | TTS-2.2 | SEQ ID NO. 4 | transport-secretion protein 2.2 | 1.5 |
| 220757_s_at | UBXD1 | SEQ ID NO. 5 | UBX domain-containing 1 | 1.5 |
| 210075_at | LOC51257 | SEQ ID NO. 6 | Hypothetical protein LOC51257 | 1.4 |
| 210740_s_a | ITRPK1 | SEQ ID NO. 7 | Inositol 1,3,4-triphosphate 5/6 | 1.3 |
| | | | kinase | |
| 209018_s_at | PINK1 | SEQ ID NO. 8 | PTEN induced putative kinase 1 | 1.5 |

A final PLS analysis involving the eight most predictive genes was applied to all 222 subjects involved in this study. The t-scores of the eight genes are able to -predict the CAD-Index very accurately. Taking into account that the CAD-Index is just a semi-quantitative estimate of stenosis which naturally implies variation across subjects even with the same extent of stenosis, the prediction based on expression pattern of the eight most predictive genes is convincing.

The differential expression pattern of the 8 predictor genes (Table 7) is confirmed be RT-PCR. The following primers were used for RT-PCR for these genes:

TABLE 8
Primers

| | | |
|---|---|---|
| Primer Name | SEQ. ID No. | Probe Sequence (5′-3′) |

| | | |
|---|---|---|
| 212788_x_at | SEQ ID. No. 1 | TCTGGAAGGCGTGAGCCACTTCTTC |
| | SEQ ID. No. 1 | GCTACGAGCGTCTCCTGAAGATGCA |
| | SEQ ID. No. 1 | AAACCCCAGACGCCATGAAAGCTGC |
| | SEQ ID. No. 1 | TGAAAGCTGCCATGGCCCTGGAGAA |
| | SEQ ID. No. 1 | CTCTGTGACTTCCTGGAGACTCACT |
| | SEQ ID. No. 1 | GGCTGGGCGAGTATCTCTTCGAAAG |
| | SEQ ID. No. 1 | TCGAAAGGCTCACTCTCAAGCACGA |
| | SEQ ID. No. 1 | AGCACGACTAAGAGCCTTCTGAGCC |
| | SEQ ID. No. 1 | GAGCCCAGCGACTTCTGAAGGGCCC |
| | SEQ ID. No. 1 | TCCCTCCAGCCAATAGGCAGCTTTC |
| | SEQ ID. No. 1 | GCAGCTTTCTTAACTATCCTAACAA |
| 40850_at | SEQ ID No. 2 | AGACCGCCTTGTACCGGAAAATGCT |
| | SEQ ID No. 2 | GCAAGGGTGCCTGGTCCATCCCATG |
| | SEQ ID No. 2 | GTGCCTGGTCCATCCCATGGAAGTG |
| | SEQ ID No. 2 | CCATCCCATGGAAGTGGCTGTTTGG |
| | SEQ ID No. 2 | TGTTTGGGGCGACTGCTGTTGCCTT |
| | SEQ ID No. 2 | ACTGAGGCCCTCTAGGAGGAAAGCC |
| | SEQ ID No. 2 | CTGAGGCCCTCTAGGAGGAAAGCCC |
| | SEQ ID No. 2 | GAGGCCCTCTAGGAGGAAAGCCCAG |
| | SEQ ID No. 2 | AGGCCCTCTAGGAGGAAAGCCCAGA |
| | SEQ ID No. 2 | GGCCCTCTAGGAGGAAAGCCCAGAG |
| | SEQ ID No. 2 | GCCCTCTAGGAGGAAAGCCCAGAGG |
| | SEQ ID No. 2 | CCTCTAGGAGGAAAGCCCAGAGGGA |
| | SEQ ID No. 2 | TAGGTCTCCGCCAGGGCTGGCCTCA |
| | SEQ ID No. 2 | AGGGCTGGCCTCAGTTTCTCCTCAA |
| | SEQ ID No. 2 | GGCTGGCCTCAGTTTCTCCTCAACA |
| | SEQ ID No. 2 | AGTTTCTCCTCAACAGGCCTGGGGG |
| 212639_x_at | SEQ ID No. 3 | GATCACCAATGCTTGCTTTGAGCCA |
| | SEQ ID No. 3 | GCTTTGAGCCAGCCAACCAGATGGT |
| | SEQ ID No. 3 | AAATGTGACCCTCGCCATGGTAAAT |
| | SEQ ID No. 3 | CCGTGGTGACGTGGTTCCCAAAGAT |
| | SEQ ID No. 3 | ATGTTAATGCTGCCATTGCCACCAT |
| | SEQ ID No. 3 | AGTTTGTGGATTGGTGCCCCACTGG |
| | SEQ ID No. 3 | CTCCCACTGTGGTGCCTGGTGGAGA |
| | SEQ ID No. 3 | GAGAGCTGTGTGCATGCTGAGCAAC |
| | SEQ ID No. 3 | GCCTTTGTTCACTGGTACGTGGGTG |
| | SEQ ID No. 3 | GGCCCGTGAAGATATGGCTGCCCTT |
| | SEQ ID No. 3 | CTAATTATCCATTCCTTTTGGCCCT |
| 39854_r_at | SEQ ID No. 4 | ATGCGCAACAACCTCTCGCTGGGGG |
| | SEQ ID No. 4 | CCGAAGCTCTGCGCATGCGCGCACC |
| | SEQ ID No. 4 | CCCCGCGGACCCAGCATCCCCGCAG |
| | SEQ ID No. 4 | CCGCGGACCCAGCATCCCCGCAGCA |
| | SEQ ID No. 4 | CCTGCTCCCGAGGCCCGGCCCGTGA |
| | SEQ ID No. 4 | GGAACCCTGCCTGAGACGCCTCCAT |
| | SEQ ID No. 4 | GAGACGCCTCCATTACCACTGCGCA |
| | SEQ ID No. 4 | ACGCCTCCATTACCACTGCGCAGTG |
| | SEQ ID No. 4 | CCACTGCGCAGTGAGATGAGGGGAC |
| | SEQ ID No. 4 | AGGGGACTCACAGTTGCCAAGAGGG |
| | SEQ ID No. 4 | ACTCACAGTTGCCAAGAGGGGTCTT |
| | SEQ ID No. 4 | CCTCCCCTGGGCCGCTGAGGCCCCG |
| | SEQ ID No. 4 | GTGCTGCCCGAGCACCTCCCCCGCC |
| | SEQ ID No. 4 | GAACTTTGCAGCTGCCCTTCCCTCC |
| | SEQ ID No. 4 | TTTGCAGCTGCCCTTCCCTCCCCGT |
| | SEQ ID No. 4 | AGAATTATTTATTTTCGCCAAAGCA |
| 220757_s_at | SEQ ID No. 5 | GGGCTGCGCAAGTACAACTACACGC |
| | SEQ ID No. 5 | GCACTTTCTACGCTCGGGAGCGGCT |
| | SEQ ID No. 5 | TGCAGAGCGACTGGCTGCCTTTTGA |
| | SEQ ID No. 5 | GGCCTCGGGAGGGCAGAAGCTGTCC |
| | SEQ ID No. 5 | TGTCCGAGGACGAGAACCTGGCCTT |
| | SEQ ID No. 5 | ACCTGGCCTTGAACGAGTGCGGGCT |
| | SEQ ID No. 5 | AGCTCCTGTCAGCCATCGAGAAGCT |
| | SEQ ID No. 5 | AAAAGCAGGGTTGGCCTCAGCCCTG |
| | SEQ ID No. 5 | ACCTCTGGAAATACTTGGCTCTGCC |
| | SEQ ID No. 5 | GCCCCATGGGCACGGGAGGGGCGCC |
| | SEQ ID No. 5 | AGCCGTGGAGCTGTGGAATTGGGCC |
| 210075_at | SEQ ID No. 6 | CAGTATGAATGCTGGGCTCTCCGGA |
| | SEQ ID No. 6 | AGAGGTAGCTGGTGATACCCTGTCC |
| | SEQ ID No. 6 | GGAAGGACTTCCACTTCAACACTTC |
| | SEQ ID No. 6 | GCACGGCCTGAACGCTTCTTAGGCC |
| | SEQ ID No. 6 | TTAGGCCAAGAGACACCATGCGGAG |
| | SEQ ID No. 6 | CATGCGGAGCCTAGTCTGTGATCCT |
| | SEQ ID No. 6 | GACATGGTCCTGAGCTCTGGACGGA |
| | SEQ ID No. 6 | TGTGGCCGGTGTATCAAGGGCGCCC |
| | SEQ ID No. 6 | TTCCAGCAAGCTTCTTGCGCTTCTC |
| | SEQ ID No. 6C | TGGCACCCTCGACTTTATATAAAA |
| | SEQ ID No. 6 | TGCACTGCGTTTCAAAAACCCACCC |
| 209018_s_at | SEQ ID No. 7 | GGCGGAAACGGCTGTCTGATGGCCC |
| | SEQ ID No. 7 | GGCTGATGCCTGGGCAGTGGGAGCC |
| | SEQ ID No. 7 | GAGCCATCGCCTATGAAATCTTCGG |
| | SEQ ID No. 7 | AGCCGCAGCTACCAAGAGGCTCAGC |
| | SEQ ID No. 7 | CTACCTGCACTGCCCGAGTCAGTGC |
| | SEQ ID No. 7 | TCAGTGCCTCCAGACGTGAGACAGT |
| | SEQ ID No. 7 | TCTGCCCGAGTAGCCGCAAATGTGC |
| | SEQ ID No. 7 | AATGTGCTTCATCTAAGCCTCTGGG |
| | SEQ ID No. 7 | CCAACAATCGGCCGCCACTTTGTTG |
| | SEQ ID No. 7 | ATGCTCTTTCTGGCTAACCTGGAGT |
| | SEQ ID No. 7 | GATGTCCCTGCATGGAGCTGGTGAA |
| 210740_s_at | SEQ ID No. 8 | CCCATCACCTTGGCAGCAAAGCACT |
| | SEQ ID No. 8 | TGCTGGGTGAGAGGCATCAGCCCCC |
| | SEQ ID No. 8 | ATCAGCCCCCACAAGTATGTTTTTG |
| | SEQ ID No. 8 | AAGTGCTGAGTGTCCCGAGAGAGGC |
| | SEQ ID No. 8 | CAGCTGGGCTGCAGGATGCCCACTT |
| | SEQ ID No. 8 | CCATCAGAACTGCCCGGCTTTTTTG |
| | SEQ ID No. 8 | ACTGAGGACCCAACAACTAACCACG |
| | SEQ ID No. 8 | CACGACTTGAGTTTTGAACCCCGAT |
| | SEQ ID No. 8 | ATTAATGTCTGTACGTCACCTTTCC |
| | SEQ ID No. 8 | AACAGGAAAGCGTGGCTGGCCTCTT |
| | SEQ ID No. 8 | TCTIGCACTGCTTTGTCTCCAAAAT |

In summary, it is investigated whether gene expression patterns in circulating leukocytes are associated with presence and extent of CAD. Patients undergoing coronary angiography were selected according to their Duke CAD index (CADi), a validated angiographical measure of the extent of coronary atherosclerosis that correlates with outcome. RNA was extracted from 120 patients with CAD (CADi>23) and from 121 partially matched controls without CAD (CADi=0). Gene expression was assessed using Affymetrix U1333A chips. Genes correlating with CAD were identified using a Spearman test, and predictive gene expression patterns were identified using a partial least squares (PLS) regression analysis.

160 individual genes were found to significantly correlate with CADi (rho>0.2, P<0.0027, n=222), although changes in individual gene expression were relatively small (1.2 to 1.5 fold). Using these 160 genes, the PLS multivariate regression model resulted in a highly predictive model (r²=0.764, P<0.001). Subsequent analysis showed that most of the predictive model was carried by only 8 genes (r²=0.752) (TABLE 7).

In conclusion, simultaneous expression pattern of eight genes is highly predictive for CAD. Peripheral leukocyte gene expression pattern is thus a non-invasive biomarker for CAD and leads to new pathophysiologic insights.

### EXAMPLE 2

Extension of the Modeling (Model 2)

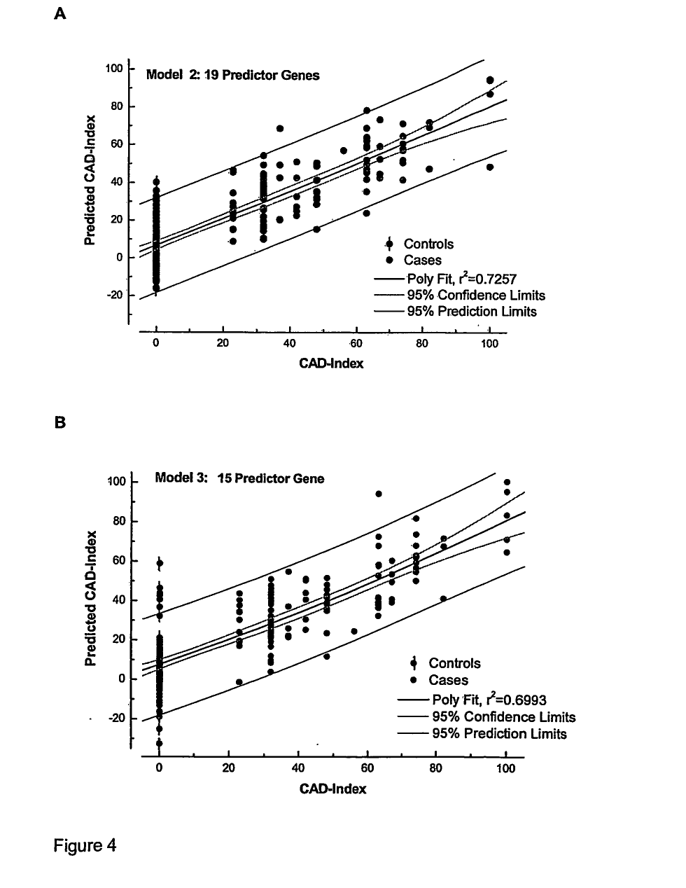

The modelling procedure is repeated with 152 candidate genes, i.e. the 160 candidate genes of Table 6 minus the eight predictive genes of Table 7 of Example 1 by using exactly the PLS methods as for model 1 (Example 1). The CAD-index as predicted by gene expression pattern of 19 genes (Table 9) versus the actual CAD-index as assessed by the clinicians are displayed in FIG. 4. r² of model 2 is only marginally less than that of model 1 (Example 1) (0.75 versus 0.72) and the 95% prediction confidence bands are comparable.

TABLE 9
The 19 best predictor genes of model 2.

| Symbol | Accession # | Gene Name | Median* | rho | SEQ ID NO |
|---|---|---|---|---|---|
| PMS2L5 | NM_174930.2 | postmeiotic | 496 | 0.24 | SEQ ID NO. 9 |
| | | segregation increased | | | |
| | | 2-like 5 | | | |
| RXRA | NM_002957 | retinoid X receptor, | 466 | 0.24 | SEQ ID NO. 10 |
| | | alpha | | | |
| GCN5L1 | NM_001487 | GCN5 general control | 540 | 0.23 | SEQ ID NO. 11 |
| | | of amino-acid | | | |
| | | synthesis 5-like 1 | | | |
| | | (yeast) | | | |
| CABIN1 | NM_012295 | calcineurin binding | 482 | 0.21 | SEQ ID NO. 12 |
| | | protein 1 | | | |
| LGALS9 | NM_002308.2, | lectin, galactoside- | 391 | 0.25 | SEQ ID NO. 13 |
| | NM_002308.2 | binding, soluble, 9 | | | |
| | | (galectin 9) | | | |
| CEBPA | NM_004364 | CCAAT/enhancer | 370 | 0.35 | SEQ ID NO. 14 |
| | | binding protein | | | |
| | | (C/EBP), alpha | | | |
| LRRN4 | NM_002319.2 | leucine rich repeat | 414 | 0.26 | SEQ ID NO. 15 |
| | | neuronal 4 | | | |
| STXBP2 | NM_006949.1 | syntaxin binding | 813 | 0.25 | SEQ ID NO. 16 |
| | | protein 2 | | | |
| SH3BP2 | NM_003023.2 | SH3-domain binding | 479 | 0.23 | SEQ ID NO. 17 |
| | | protein 2 | | | |
| RNF24 | NM_007219.2 | ring finger protein 24 | 918 | 0.28 | SEQ ID NO. 18 |
| PLAUR | NM_002659.1 | plasminogen | 275 | 0.20 | SEQ ID NO. 19 |
| | | activator, urokinase | | | |
| | | receptor | | | |
| RIS1 | NM_015444.1 | Ras-induced | 84 | 0.28 | SEQ ID NO. 20 |
| | | senescence 1 | | | |
| ADD1 | NM_001119.3, | adducin 1 (alpha) | 464 | 0.21 | SEQ ID NO. 21 |
| | NM_001119.3, | | | | |
| | NM_176801.1, | | | | |
| | NM_014190.2 | | | | |
| GPSM3 | NM_022107.1 | G-protein signalling | 551 | 0.27 | SEQ ID NO. 22 |
| | | modulator 3 (AGS3- | | | |
| | | like, C. elegans) | | | |
| BC002942 | NM_033200.1 | hypothetical protein | 381 | 0.28 | SEQ ID NO. 23 |
| | | BC002942 | | | |
| TNFRSF5 | NM_001250.3, | tumor necrosis factor | 408 | 0.22 | SEQ ID NO. 24 |
| | NM_001250.3 | receptor superfamily, | | | |
| | | member 5 | | | |
| N4BP1 | Hs.323712 | Nedd4 binding protein 1 | 833 | 0.21 | SEQ ID NO. 25 |
| | (Unigene #) | | | | |
| FLJ12438 | NM_021933.1 | hypothetical protein | 555 | 0.22 | SEQ ID NO. 26 |
| | | FLJ12438 | | | |
| MMP24 | NM_006690.2 | matrix | 820 | 0.20 | SEQ ID NO. 27 |
| | | metalloproteinase 24 | | | |
| | | (membrane-inserted) | | | |

*median refers to the median intensity of the signal and rho is Spearman's rank correlation of the respective gene with CAD-Index.

### EXAMPLE 3

Extension of the Modeling (Model 3)

In a third modelling approach the remaining 133 genes, i.e. the 160 candidate genes (Table 6) minus the eight predictive genes (Table 7; Example 1) of model 1, minus the nineteen predictor genes (Table 9; Example 2) of model 2 are subjected to partial least square regression as described in Example 1. The result is depicted in FIG. 4 and the corresponding 15 best predictor genes are compiled in Table 10.

TABLE 10
The 15 best predictor genes of model 3.

| Symbol | Accession # | Gene Name | Median* | rho | SEQ ID NO |
|---|---|---|---|---|---|
| PTP4A1 | NM_003463.2 | protein tyrosine | 62 | 0.27 | SEQ ID NO. 28 |
| | | phosphatase type | | | |
| | | IVA, member 1 | | | |
| PAFAH1B1 | NM_000430.2 | platelet-activating | 204 | 0.27 | SEQ ID NO. 29 |
| | | factor | | | |
| | | acetylhydrolase, | | | |
| | | isoform lb, alpha | | | |
| | | subunit | | | |
| SOX4 | NM_003107.2 | SRY (sex determining | 54 | 0.20 | SEQ ID NO. 30 |
| | | region Y)-box 4 | | | |
| ASNA1 | NM_004317.1 | arsA arsenite | 360 | 0.21 | SEQ ID NO. 31 |
| | | transporter, ATP- | | | |
| | | binding, homolog 1 | | | |
| | | (bacterial) | | | |
| MAN2A2 | NM_006122.1, | mannosidase, alpha, | 358 | 0.28 | SEQ ID NO. 32 |
| | NM_006122.1 | class 2A, member 2 | | | |
| NFYC | NM_014223.2 | nuclear transcription | 238 | 0.30 | SEQ ID NO. 33 |
| | | factor Y, gamma | | | |
| NOTCH2 | NM_024408.2 | Notch homolog 2 | 87 | 0.20 | SEQ ID NO. 34 |
| | | (Drosophila) | | | |
| HDAC5 | NM_005474.3, | histone deacetylase 5 | 400 | 0.28 | SEQ ID NO. 35 |
| | NM_005474.3 | | | | |
| HCFC1 | NM_005334.1 | host cell factor C1 | 15 | 0.23 | SEQ ID NO. 36 |
| | | (VP16-accessory | | | |
| | | protein) | | | |
| NFX1 | NM_002504.3, | nuclear transcription | 82 | 0.26 | SEQ ID NO. 37 |
| | NM_002504.3, | factor, X-box binding 1 | | | |
| | NM_147133.1 | | | | |
| CRSP2 | NM_004229.2 | cofactor required for | 35 | 0.24 | SEQ ID NO. 38 |
| | | Sp1 transcriptional | | | |
| | | activation, subunit 2a | | | |
| ICAM1 | NM_000201.1 | intercellular adhesion | 307 | 0.21 | SEQ ID NO. 39 |
| | | molecule 1 (CD54), | | | |
| | | human rhinovirus | | | |
| | | receptor | | | |
| PSG3 | NM_021016.2 | pregnancy specific | 17 | 0.22 | SEQ ID NO. 40 |
| | | beta-1-glycoprotein 3 | | | |
| STC2 | NM_003714.1 | stanniocalcin 2 | 80 | 0.24 | SEQ ID NO. 41 |
| SEMA3C | NM_006379.2 | sema domain, | 71 | 0.28 | SEQ ID NO. 42 |
| | | immunoglobulin | | | |
| | | domain (lg), | | | |
| | | (semaphorin) 3C | | | |

*median refers to the median intensity of the signal and rho is Spearman's rank correlation of the respective gene with CAD-Index

In conclusion, due to the high information content of the 160 candidate genes it is possible to generate three models that may predict the extent of coronary artery disease based on gene expression pattern. By applying the three models for example in parallel to new unknown samples the likelihood of correct prediction may increase dramatically. Furthermore, the robustness of the predictive model may be increased; i.e. if for some technical or biological reason the genes being involved in one model are of poor quality there are still two more predictive models that can be used for prediction.

Further information on the genes of Tables 7 and/or Table 9 and/or Table 10 such as Unigene #, Probeset # and/or Accession # is listed in Table 6.

### EXAMPLE 4

Proteomic Discovery of Coronary Artery Disease Proteins using Industrial Scale Analysis of Pooled Plasma.

Established are male populations of 53 patients with angiographic coronary artery disease (defined as at least one lesion with > or = to 50% stenosis) and 53 patients with no angiographic coronary disease from the Duke Databank for Cardiovascular Disease. For a description of the Duke Databank for Cardiovascular Disease, see Allen LaPointe, Nancy M. et al., Journal of the Amercan College of Cardiology 41 (Suppl A):517A (2003). These patients were matched for age and race and extremes of risk factors and major plasma protein abnormalities were removed in prescreening. Plasma samples of each group were pooled to make large volumes (6 liters each) to identify low abundance proteins, in the picomolar range. After specific removal of albumin and immunoglobulins, and enrichment of smaller proteins (<20-40 kDa), samples were separated into 12,960 fractions by liquid chromatography, and analyzed by mass spectrometry (LC-ESI MS/MS and MALDI-TOF), before and after enzymatic digestion. See, Rose, Keith et al., Proteomics (2004) (DOI 10.1002/pmic.200300718).

731 plasma proteins or fragments were identified, including low abundance moieties such as leptin and bradykinin. 17 were well detected and strongly differentially displayed according to disease state. The proteins in categories are summarized in the following TABLE 11 with their parent accession number:

TABLE 11

| | Accession | SEQ ID NO. |
|---|---|---|
| Disease > Control | | |
| Fibrinogen Gamma Chain | P02679 | SEQ ID NO. 43 |
| Mature Form of Collagen alpha 3(VI) | P12111 | SEQ ID NO. 44 |
| chain | | |
| Mature Form of Complement C1s | P09871 | SEQ ID NO. 45 |
| (C1 esterase) | | |
| CD59 | P13987 | SEQ ID NO. 46 |
| Insulin like growth factor binding complex | P35858 | SEQ ID NO. 47 |
| acid labile chain (ALS) | | |
| Defensin 5 | Q01523 | SEQ ID NO. 48 |
| Proline Rich Acidic Protein | Q96NZ9 | SEQ ID NO. 49 |
| Emilin-3 | Q9H8L6 | SEQ ID NO. 50 |
| CA11 Protein | Q9NS71 | SEQ ID NO. 51 |
| Predominant in Disease | | |
| C5A Anaphylotoxin | P01031 | SEQ ID NO. 52 |
| Nonsecretory ribonuclease isoform | P10153 | SEQ ID NO. 53 |
| (three moieties) | | |
| Control > Disease | | |
| Glutathione transferase omega 1 | P78417 | SEQ ID NO. 54 |
| Complement factor H-related protein 1 | Q03591 | SEQ ID NO. 55 |
| (FHR-1) | | |
| Secreted phosphoprotein 24 (SPP-24) | Q13103 | SEQ ID NO. 56 |
| Predominant in Controls | | |
| Mature form of chitotriosidase | Q13231 | SEQ ID NO. 57 |

Additional information about peptide Q96NZ9 is provided in PCT patent applications WO 02/00690 and WO 02/08284 (PROL 195-SEQ ID 212). Additional information about peptide Q9NS71 is provided in PCT patent applications WO02/00690 and WO02/08284 (PRO1005 -SEQ ID 140).

In conclusion, this systematic and comprehensive approach has identified a large number of proteins that are differentially displayed in populations with and without coronary disease. These proteins include inflammatory mediators and defense mechanism proteins and now comprise a group of candidates for additional validation tests to identify novel markers for disease.

1-9. (canceled)

10. A method of identifying or predicting coronary artery disease (CAD) in a subject comprising:
(a) determining the level of one or more peptides selected from Table 11 in a subject to provide a first value,
(b) determining the level of said one or more peptides selected from Table 11 in a control or reference standard to provide a second value and
(c) comparing whether there is a difference between said first value and second value.

11. A method according to claim 10 wherein the compared peptide level is increased in an identification or prediction of coronary artery disease for those peptides of Table 11 wherein Disease>Control or for those peptides of Table 11 wherein the peptide is Predominant in Disease.

12. A method according to claim 11 wherein the compared peptide level is decreased in an identification or prediction of coronary artery disease for those peptides of Table 11 wherein Control>Disease or for those peptides of Table 11 wherein the peptide is Predominant in Control.

13-25. (canceled)

26. A method according to claim 10 wherein the peptide level of a plurality of peptides selected from Table 11 is determined.

27. A method according to claim 10 wherein the peptide level of all peptides selected from Table 11 are determined.

28. A method according to claim 10 wherein said peptide level is measured in a blood, plasma or serum sample.

29. (canceled)

30. A method according to claim 10 wherein the amount of said protein expression product and/or the amount of peptide is detected using an antibody, antibody derivative or antibody fragment, which specifically binds to the protein.

31-33. (canceled)

34. A method according to claim 10 wherein the level of peptide is measured ex vivo in a sample selected from the group of: blood, serum, plasma, lymph, urine, tear, saliva, cerebrospinal fluid, leukocyte sample or tissue sample.

35-59. (canceled)

60-88. (canceled)
